(19) United States
(12) Patent Application Publication
(10) Pub. No.: US 2024/0368620 A1
(43) Pub. Date: Nov. 7, 2024

(54) BLACKLEG RESISTANT PLANTS AND METHODS FOR THE IDENTIFICATION OF BLACKLEG RESISTANT PLANTS

(71) Applicant: BASF Agricultural Solutions Seed US LLC, Florham Park, NJ (US)

(72) Inventors: Steven ENGELEN, Lochristi (BE); Michel VAN THOURNOUT, Gent (BE)

(21) Appl. No.: 18/290,666

(22) PCT Filed: Jul. 22, 2022
(86) PCT No.: PCT/US2022/074073

§ 371 Date: Jan. 19, 2024

(30) Foreign Application Priority Data
Jul. 23, 2021 (EP) 21187390.6

(51) Int. Cl. C12N 15/82; A01H 1/04; A01H 6/20; G01N 33/00

(52) U.S. Cl. CPC C12N 15/8281; A01H 1/045; A01H 6/20; C12N 15/8275; G01N 33/0098

(57) ABSTRACT

The present relates to methods for producing blackleg resistant plants as well as to methods for the identification of blackleg resistant plants. Further provided are blackleg resistant plants as well as kits for assessing blackleg resistance in a plant.

The present relates to methods for producing blackleg resistant plants and to methods for the identification of blackleg resistant plants. Further provided are blackleg resistant plants as well as kits for assessing blackleg resistance in a plant.

### BACKGROUND OF THE INVENTION

Blackleg or stem canker is a major disease of Brassica napus L. (oilseed rape or Canola), causing annually major economic losses worldwide, in particular in Europe, Australia and North America. Blackleg is caused by the fungal pathogen Leptosphaeria maculans (anamorph Phoma lingam Tode ex. Fr.). L. maculans symptoms can develop on cotyledons, leaves, pods and stems. Leaf lesions develop after infection by wind dispersed ascospores and/or water (splash) dispersed conidiospores. Stem symptoms (or cankers) can arise through direct infection of the stems or through systemic growth of the fungus from leaf lesions, through the vascular tissue into the stem (Hammond et al. (1985), Plant Pathology 34:557-565). Stem cankers may girdle the stem, which can lead to the lodging of plants and plant death. Less severe cankers can cause a restriction in water and nutrient flow, which in turn may lead to shriveling of seeds and pods. Pod infection can lead to premature podshatter and seed infection.

The incorporation of blackleg resistance into B. napus cultivars is one of the major objectives in breeding programs worldwide. Although both the spraying of fungicides and cultural practices are used to reduce yield losses caused by blackleg infection, the most reliable method of control to date is genetic resistance. Brassica napus (2n=38, genome AACC) is an amphidiploid species, which originated from a spontaneous hybridization of Brassica rapa L. (syn. B. campestris; 2n=20, AA) and Brassica oleracea L. (2n=18, CC). B. napus contains the complete chromosome sets of these two diploid genomes.

Plant resistance is a powerful tool to combat blackleg disease. Blackleg resistance is assessed either in glasshouse or in field experiments. Further, it can be assessed at different stages of the plant development. When referring to blackleg resistance, normally different types of resistance are therefore distinguished depending on the plant stage and tissue assessed, such as seedling resistance (“early” resistance) and adult plant resistance (‘late’ or ‘stem’ resistance). Plant tissues analyzed for resistance are for example cotyledons, leaves and stem bases. Genetical resistance to blackleg has been reported to be either monogenic (under control of a major gene) or polygenic (under control of several minor genes).

A number of resistance loci have been mapped in B. napus. Resistance against the hemibiotrophic fungal pathogen Leptosphaeria maculans is governed largely by race-specific R genes. More than 15 R genes, identified in several Brassica species, have been reported to convey race-specific resistance against L. maculans. Twelve of these R genes have been positioned in the A genome of B. napus or B. rapa (Rlm1, Rlm2, Rlm3, Rlm4, Rlm7, Rlm9, LepR1, LepR2, LepR3, LepR4) or the B genome of B. juncea (LmJR1, LmJR2) via linkage mapping (Larkan et al. (2016) Single R Gene Introgression Lines for Accurate Dissection of the Brassica-Leptosphaeria Pathosystem. Front. Plant Sci. 7:1771. doi: 10.3389/fpls.2016.01771). Rlm3, Rlm4, Rlm7 and Rlm9 are suspected to be allelic R genes which interact with L. maculans avirulence genes AvrLm3 (Rlm3), AvrLm4-7 (Rlm4 and Rlm7) and AvrLm5-9 (Rlm9) (Dolatabadian et al. (2022) Canadian Journal of Plant Pathology, 44:2, 157-190).

U.S. Pat. No. 7,893,325 describes a Brassica napus plant, comprising on chromosome 8 a Leptosphaeria maculans resistance gene derived from Brassica rapa, wherein said resistance gene is associated with AFLP markers E32/M50-M362 and P34/M48-M283 on chromosome 8.

WO 2008/101343 A1 describes a method of conferring blackleg resistance to a plant comprising: introducing a nucleic acid molecule comprising a nucleic acid molecule encoding LepR3.

WO 2015/038469 A1 describes molecular markers for blackleg resistance gene Rlm2 in Brassica napus.

WO 2015/038470 A1 describes molecular markers for blackleg resistance gene Rlm4 in Brassica napus.

WO 2020/036950 A1 describes molecular markers for blackleg resistance gene Rlm1 in Brassica napus.

WO2020/036954 A1 describes molecular markers for blackleg resistance gene Rlm7 in Brassica napus.

The NCBI reference sequence XP_013589432.1 is putative cell wall-associated receptor kinase-like 10 (partial) from Brassica oleracea.

It is desirable to identify new genetic sources of resistance, methods for transferring these into varieties with high agronomic performance and methods for enhancing durability of resistance.

The present invention, including the different embodiments provided in the specification and claims, provides plants comprising a blackleg resistance gene, Rlm3, and methods and means for transferring Rlm3 into plants as well as methods of detecting the presence/absence of Rlm3 in plants.

### SUMMARY OF THE INVENTION

The present invention provides a method for producing a blackleg resistant plant comprising the step of introducing into the genome of a plant a polynucleotide comprising a blackleg resistance locus Rlm3, wherein said Rlm3 locus comprises at least one polynucleotide having a nucleic acid sequence selected from the group consisting of:
a) a nucleic acid sequence as shown in any one of SEQ ID NOs: 1, 4, 42 and 44; and
b) a nucleic acid sequence which is at least 80%, at least 85%, at least 90%, at least 95%, at least 96%, at least 97%, at least 98% or at least 99% identical to the nucleic acid sequence shown in any one of SEQ ID NOs: 1, 4, 42 and 44, and
c) a nucleic acid sequence having at least 80%, or at least 85%, or at least 90%, or at least 92%, or at least 95%, or at least 98%, or at least 99%, or 100% sequence identity to nt 2106 to 9496 of SEQ ID NO: 1, to nt 2106-4688 of SEQ ID NO: 1, or to nt 5908-9496 of SEQ ID NO: 1

The present invention further provides a method for producing a blackleg resistant plant comprising the step of introducing into the genome of a plant at least one polynucleotide comprising an Rlm3 associated open reading frame, wherein said Rlm3 associated open reading frame encodes a polypeptide comprising an amino acid sequence selected from the group consisting of:
a) an amino acid sequence as shown in any one of SEQ ID NOs: 5, 43 and 45;
b) an amino acid sequence which is at least 80%, at least 85%, at least 90%, at least 95%, at least 96%, at least 97%, at least 98% or at least 99% identical to the amino acid sequence shown in any one of SEQ ID NOs: 5, 43 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOS: 1, 4, 42 and 44, wherein said polypeptide is capable of conferring blackleg resistance to a plant;
d) a partial sequence of any one of a) to c), wherein said polypeptide is capable of conferring blackleg resistance to a plant.

In one embodiment, the above methods of the present invention further comprise
i) identifying a plant having integrated into its genome said polynucleotide; and
ii) generating progeny from said plant wherein blackleg resistance has been conferred to said progeny.

The present invention also provides a method of conferring blackleg resistance to a plant comprising the step of genetically modifying a silent allele of a gene encoding a polypeptide comprising a Rlm3 associated open reading frame such that the silent allele is capable of expressing said polypeptide, wherein said Rlm3 associated open reading frame encodes a polypeptide comprising an amino acid sequence selected from the group consisting of:
a) an amino acid sequence as shown in any one of SEQ ID NOs: 5, 43 and 45;
b) an amino acid sequence which is at least 80%, at least 85%, at least 90%, at least 95%, at least 96%, at least 97%, at least 98% or at least 99% identical to the amino acid se-quence shown in any one of SEQ ID NOs: 5, 43 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOS: 1, 4, 42 and 44, wherein said polypeptide is capable of conferring blackleg resistance to a plant;
d) a partial sequence of any one of a) to c), wherein said polypeptide is capable of conferring blackleg resistance to a plant.

In an embodiment of the aforementioned method, the silent allele is modified by homologous recombination or by genome editing technology.

The present invention also contemplates a method for producing a blackleg resistant plant comprising the step of introducing into the genome of a plant at least one polynucleotide comprising a Rlm3 associated open reading frame, wherein said Rlm3 associated open reading frame encodes a polypeptide comprising an amino acid sequence selected from the group consisting of:
a) an amino acid sequence as shown in any one of SEQ ID NOs: 5, 43 and 45;
b) an amino acid sequence which is at least 80%, at least 85%, at least 90%, at least 95%, at least 96%, at least 97%, at least 98% or at least 99% identical to the amino acid se-quence shown in any one of SEQ ID NOs: 5, 43 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 4, 42 and 44, wherein said polypeptide is capable of conferring blackleg resistance to a plant;
d) a partial sequence of any one of a) to c), wherein said polypeptide is capable of conferring blackleg resistance to a plant.

In one embodiment of the above method, homologous recombination or a genome editing technology is used.

Moreover, the present invention relates to a method for the manufacture of food (such as oil, a meal, starch, flour, or a protein), feed (such as an oil, a meal, starch, flour, or a protein) or an industrial product (such as a biofuel, a fiber, an industrial chemical, a drug or a nutrient) comprising:
i) conferring blackleg resistance to a plant by the method of the present invention and
ii) preparing the food, feed or industrial product from the plant obtained in step i).

The present invention further relates to a method for assessing blackleg resistance in a plant comprising the steps of:
I) determining the presence or absence of a blackleg resistance locus Rlm3 or a polynucleotide comprising a Rlm3 associated open reading frame in a sample of said plant comprising genomic DNA, wherein
   i) said Rlm3 locus comprises at least one polynucleotide having a nucleic acid sequence selected from the group consisting of:
      a) a nucleic acid sequence as shown in any one of SEQ ID NOs: 1, 4, 42 and 44; and
      b) a nucleic acid sequence which is at least 80, at least 85%, at least 90%, at least 95%, at least 96%, at least 97%, at least 98% or at least 99% identical to the nucleic acid sequence shown in any one of SEQ ID NOs: 1, 4, 42 and 44;
      c) a nucleic acid sequence having at least 80%, or at least 85%, or at least 90%, or at least 92%, or at least 95%, or at least 98%, or at least 99%, or 100% sequence identity to nt 2106 to 9496 of SEQ ID NO: 1, to nt 2106-4688 of SEQ ID NO: 1, or to nt 5908-9496 of SEQ ID NO: 1 and
   ii) said Rlm3 associated open reading frame encodes a polypeptide comprising an amino acid sequence selected from the group consisting of:
      a) an amino acid sequence as shown in any one of SEQ ID NOs: 5, 43 and 45;
      b) an amino acid sequence which is at least 80%, at least 85%, at least 90%, at least 95%, at least 96%, at least 97%, at least 98% or at least 99% identical to the amino acid sequence shown in any one of SEQ ID NOs: 5, 43 and 45, wherein said polypeptide is capable of conferring blackleg resistance to a plant;
      c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 4, 42 and 44, wherein said polypeptide is capable of conferring blackleg resistance to a plant;
      d) a partial sequence of any one of a) to c), wherein said polypeptide is capable of conferring blackleg resistance to a plant;
      and
II) assessing blackleg resistance of the plant based on the presence or absence of said blackleg resistance locus Rlm3 in said plant.

The present invention also encompasses a method for assessing blackleg resistance in a plant comprising the steps of:
i) determining the presence or absence of a polypeptide encoded by a Rlm3 associated open reading frame in a sample of said plant comprising protein, wherein said Rlm3 associated open reading frame encodes a polypeptide comprising an amino acid sequence selected from the group consisting of:
   a) an amino acid sequence as shown in any one of SEQ ID NOs: 5, 43 and 45;
   b) an amino acid sequence which is at least 80%, at least 85%, at least 90%, at least 95%, at least 96%, at least 97%, at least 98% or at least 99% identical to the amino acid sequence shown in any one of SEQ ID NOs: 5, 43 and 45, wherein said polypeptide is capable of conferring blackleg resistance to a plant;
   c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 4, 42 and 44, wherein said polypeptide is capable of conferring blackleg resistance to a plant;
   d) a partial sequence of any one of a) to c), wherein said polypeptide is capable of conferring blackleg resistance to a plant;
   and
ii) assessing blackleg resistance of the plant based on the presence or absence of said polypeptide encoded by a Rlm3 associated open reading frame.

Moreover, the present invention relates the use of the blackleg resistance locus Rlm3 for assessing blackleg resistance or a polynucleotide comprising a Rlm3 associated open reading frame in a plant, wherein
i) said Rlm3 locus comprises at least one polynucleotide having a nucleic acid sequence selected from the group consisting of:
   a) a nucleic acid sequence as shown in any one of SEQ ID NOs: 1, 4, 42 and 44,
   b) a nucleic acid sequence which is at least 80, at least 85%, at least 90%, at least 95%, at least 96%, at least 97%, at least 98% or at least 99% identical to the nucleic acid sequence shown in any one of SEQ ID NOs: 1, 4, 42 and 44; and
   c) a nucleic acid sequence having at least 80%, or at least 85%, or at least 90%, or at least 92%, or at least 95%, or at least 98%, or at least 99%, or 100% sequence identity to nt 2106 to 9496 of SEQ ID NO: 1, to nt 2106-4688 of SEQ ID NO: 1, or to nt 5908-9496 of SEQ ID NO: 1, and
ii) said Rlm3 associated open reading frame encodes a polypeptide comprising an amino acid sequence selected from the group consisting of:
   a) an amino acid sequence as shown in any one of SEQ ID NOs: 5, 43 and 45;
   b) an amino acid sequence which is at least 80%, at least 85%, at least 90%, at least 95%, at least 96%, at least 97%, at least 98% or at least 99% identical to the amino acid sequence shown in any one of SEQ ID NOs: 5, 43 and 45, wherein said polypeptide is capable of conferring blackleg resistance to a plant;
   c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 4, 42 and 44, wherein said polypeptide is capable of conferring blackleg resistance to a plant;
   d) a partial sequence of any one of a) to c), wherein said polypeptide is preferably capable of conferring blackleg resistance to a plant.

The present invention further relates to a polynucleotide comprising a nucleic acid sequence selected from the group consisting of:
a) a nucleic acid sequence as shown in any one of SEQ ID NOs: 1, 4, 42 and 44; and
b) a nucleic acid sequence which is at least 80, at least 85%, at least 90%, at least 95%, at least 96%, at least 97%, at least 98% or at least 99% identical to the nucleic acid sequence shown in any one of SEQ ID NOs: 1, 4, 42 and 44, and
c) a nucleotide sequence having at least 80%, or at least 85%, or at least 90%, or at least 92%, or at least 95%, or at least 98%, or at least 99%, or 100% sequence identity to nt 2106 to 9496 of SEQ ID NO: 1, to nt 2106-4688 of SEQ ID NO: 1, or to nt 5908-9496 of SEQ ID NO: 1

Moreover, the present invention envisages a polynucleotide encoding a polypeptide comprising an amino acid sequence selected from the group consisting of:
a) an amino acid sequence as shown in any one of SEQ ID NOs: 5, 43 and 45;
b) an amino acid sequence which is at least 80%, at least 85%, at least 90%, at least 95%, at least 96%, at least 97%, at least 98% or at least 99% identical to the amino acid sequence shown in any one of SEQ ID NOs: 5, 43 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOS: 1, 4, 42 and 44, wherein said polypeptide is capable of conferring blackleg resistance to a plant;
d) a partial sequence of any one of a) to c), wherein said polypeptide is capable of conferring blackleg resistance to a plant.

In an embodiment of the present invention, the polynucleotide is operably linked to a heterologous promoter.

The present invention further relates to an oligonucleotide which specifically hybridizes to the polynucleotide and which is capable of being used as a primer or probe.

The present invention also relates to vector or gene construct comprising the polynucleotide of the present invention.

The present invention also relates to a host cell comprising the polynucleotide of the present invention or the vector or gene construct of the present invention.

The present invention also relates to a plant comprising the polynucleotide of the invention, vector or gene construct of the present invention, the host cell of the present invention or a plant cell which is obtainable by the methods of the present invention. In a further embodiment, this plant comprises in its genome at least one additional blackleg resistance gene selected from the group consisting of Rlm4, Rlm7 and Rlm9.

The present invention also relates to a polypeptide encoded by the polynucleotide of the present invention.

The present invention also relates to an antibody which specifically recognizes the polypeptide of the present invention.

The present invention also relates to a kit for assessing blackleg resistance in a plant comprising the oligonucleotide of the present invention or the antibody of the present invention.

Moreover, the present invention relates to a method to determine the presence or absence of a Rlm3 polynucleotide of the present invention in a biological sample (such as a plant tissue sample), comprising providing DNA from said biological sample, and analyzing said DNA for the presence or absence of said Rlm3 polynucleotide.

Moreover, the present invention relates to a method to determine the presence or absence of a Rlm3 polypeptide of the present invention in a biological sample (such as a plant tissue sample), comprising providing polypeptides from said biological sample, and analyzing said polypeptides for the presence or absence of said Rlm3 polypeptide.

Also provided is a method for protecting cultivated plants in a field, wherein said plants are plants according to the present invention, said plants further comprising at least one resistance gene conferring herbicide tolerance and wherein said method comprises applying the said herbicide to the cultivated plants in order to control weeds.

In an embodiment, the herbicide is glufosinate, glufosinate ammonium or glyphosate.

### BRIEF DESCRIPTION OF THE DRAWINGS

FIG. 1: Measurement of disease resistance as GFP fluorescence (RFU) in Brassica napus protoplats: 1: GFP control cells; 2: Cells expressing AvrLm3-WT, 3: Cells expressing AvrLm3-SP.

FIG. 2: Visualization of disease resistance as GFP fluorescence in Brassica napus protoplast: 1: GFP control cells; 2: Cells expressing AvrLm3-WT.

### DETAILED DESCRIPTION OF THE PRESENT INVENTION

The current invention is based on the identification of the Rlm3 blackleg resistance gene in Brassica. The gene encodes for a polypeptide showing homology to WAKL10 (Wall-associated receptor kinase-like 10) proteins. Specifically, three open reading frames were identified that are involved in blackleg resistance. The identification of the Rlm3 blackleg resistance gene is advantageous since it allows for the production of plants, such as transgenic plants, which are resistant to blackleg disease. Furthermore, the Rlm3 blackleg resistance gene, or a polypeptide encoded by said gene can be used as marker for the identification of plants which are resistant to blackleg disease. Moreover, a detection agent that recognizes said blackleg resistance gene (such as a primer or probe which binds to the gene) or said polypeptide (such as an antibody) can be used for identifying plants which are resistant to blackleg disease.

The genomic sequence of the identified Rlm3 blackleg resistance gene is shown in SEQ ID NO: 1. The Rlm3 blackleg resistance gene has different splice variants: One longer variant and two shorter ones.

The coding sequence of the longest open reading frame is shown in SEQ ID NO: 42 (herein referred to as Rlm3-19, or first open reading frame). The cDNA sequence is shown in SEQ ID NO: 46. The amino acid sequence of the Rlm3-19 polypeptide is shown in SEQ ID NO: 43.

The coding sequence of the second open reading frame is shown in SEQ ID NO: 4 (herein referred to as Rlm3-19a). The cDNA sequence is shown in SEQ ID NO: 9. The amino acid sequence of the Rlm3-19a polypeptide is shown in SEQ ID NO: 5.

The coding sequence of the third open reading frame is shown in SEQ ID NO: 44 (herein referred to as Rlm3-19b). The cDNA sequence is shown in SEQ ID NO: 47. The amino acid sequence of the Rlm3-19b polypeptide is shown in SEQ ID NO: 45.

As outlined herein below, the above genomic region, coding sequences, cDNA and polypeptides can be used for the generation of blackleg resistant plants by introduction of the genes into plants, such as by crossing or by transformation. Further, the genes can be used as a marker for identifying blackleg resistant plants.

The invention relates to sequences of the Rlm3 blackleg resistance locus, and sequences encoding a protein conferring resistance to blackleg resistance in Brassicaceae. The protein may comprise the amino acid sequence having at least 80% sequence identity to SEQ ID NO: 43, to SEQ ID NO: 5, or to SEQ ID NO: 45, or a functional fragment of these amino acid sequences.

It is a first embodiment of the invention to provide a Brassicaceae plant or plant cell comprising a Rlm3 blackleg resistance gene as transgene, wherein said Rlm3 blackleg resistance gene comprises a coding sequence having at least 80% sequence identity to SEQ ID NO: 42, SEQ ID NO: 4, or SEQ ID NO: 44; or encodes a protein having an amino acid sequence having at least 80% sequence identity to SEQ ID NO: 43, SEQ ID NO: 5, SEQ ID NO: 45.

In a further embodiment, said Rlm3 blackleg resistance gene comprises a sequence having at least 80% sequence identity to SEQ ID NO: 1. For example, said Rlm3 blackleg resistance gene comprises a sequence having at least 80% sequence identity to nt 2106-9496 of SEQ ID NO: 1. This region comprise the coding sequence of gene19, see Examples. Alternatively, said Rlm3 blackleg resistance gene comprises a sequence having at least 80% sequence identity to nt 2106-4688 of SEQ ID NO: 1. This region comprise the coding sequence of gene19a. Alternatively, said Rlm3 blackleg resistance gene comprises a sequence having at least 80% sequence identity to nt 5908-9496 of SEQ ID NO: 1. This region comprise the coding sequence of gene19b.

A “Rlm3 protein” or “Rlm3 polypeptide”, as used herein, is a protein encoded by a Rlm3 blackleg resistance gene. A Rlm3 protein can have an amino acid sequence having at least 80%, or at least 85%, or at least 90%, or at least 92%, or at least 95%, or at least 98%, or at least 99%, or 100% sequence identity to SEQ ID NO: 43, to SEQ ID NO: 5, or to SEQ ID NO: 45.

A “functional fragment” of the amino acid sequence having at least 80% sequence identity to SEQ ID NO: 43, denotes a protein or peptide comprising a stretch or partial sequence of the amino acid sequences as depicted above which still exerts the desired function, i.e. which increases blackleg resistance when present in a Brassicaceae plant. An assay for determining of whether a functional fragment increases blackleg resistance is provided herein in the Examples section, see Example 2.

“Blackleg” as used herein refers to the disease caused by the fungal pathogen Leptosphaeria maculans or Phoma lingam (anamorph). The definition encompasses both Tox⁰ and Tox⁺ isolates, irrespective of whether these may be found to belong to different species in later taxonomic studies.

L. maculans isolates can be classified into different pathogenicity groups (PG), depending on their specific interactions with B. napus cultivars Westar, Galcier and Quinta (Mengistu et al. (1991), Plant Disease 75:1279-1282). PG4 isolates cause sporulating lesions on all three cultivars, while PG3 isolates cause a resistance reaction on cotyledons of Quinta, and PG2 isolates cause a resistance reaction on cotyledons of Quinta and Glacier. PG1 isolates are nonpathogenic on these hosts. PG2, PG3 and PG4 isolates are also referred to as ‘highly aggressive’ or ‘highly virulent’ or ‘strongly pathogenic’ isolates, while PG1 isolates are referred to as ‘non-aggressive’ or ‘non-virulent’ or ‘weakly pathogenic’ in the literature, some-times the highly aggressive group is also termed “A” while the weakly aggressive group is termed “NA” (Badawy and Hoppe (1989), J Phytopathology 127:146-157). The highly aggressive group is distinguished from the weakly aggressive group by its production of toxins (Tox⁺ isolates vs Tox⁰ isolates). Tox⁰ isolates have been found to cause necrosis of the pith, unaccompanied by external symptoms, and it has been suggested that the effect on yield loss caused by Tox⁰ isolates has been underestimated (Johnson and Lewis (1994), Plant Pathology 43:269-277). Tox⁰ isolates are further distinguished into three groups, NA1, NA2 and NA3 and it has been suggested that NA1 isolates are predominant in Europe and NA2 isolates are more important in Canada (Gall et al. (1995), Mycol Res 99:221-229).

A “Rlm3 blackleg resistance gene”, or “R/m3 resistance gene”, or “Rlm3 gene”, as used herein, is a gene that confers enhanced resistance to Leptosphaeria maculans compared to a plant lacking the resistance gene(s) or having a non-functional (or inactivated) form of the gene(s). Thus, the gene shall confer resistance to Leptosphaeria maculans, in particular to Leptosphaeria maculans strain harboring the AvrLm3 avirulence gene, such as Isolate Lm1033-1. This resistance gene can be transferred to different varieties of B. napus, and even to different species of Brassica plants, e.g. B. juncea, e.g., using the molecular markers of this invention.

“Enhanced resistance” of plants comprising a certain resistance gene refers to a reduction in damage caused by fungal infection (such as with Leptosphaeria maculans) compared to damage caused on control plants. Damage can be assessed as, for example, the number and size of leaf symptoms, frequency and severity of stem symptoms, lodging of plants due to stem infection, etc. In particular, the reduction in damage is manifested in a reduced yield loss when plants are grown under disease pressure in the field, compared to control plants. Such reduction in yield loss can, for example, be due to the fact that the infection, reproduction, spread or survival of the fungus is reduced or prevented in plants with enhanced resistance. Enhanced resistance may also refer to plants that are completely resistant, i.e., plants on which no disease symptoms are found or plants which get the highest resistance scores in available blackleg scoring or rating assays, e.g., Khangura et al. (2003, Department of Agriculture, Western Australia, Farmnote No. 6/2003, ISSN 0726-934 x).

In an embodiment, the Rlm3 blackleg resistance gene as referred to herein confers resistance to one or more L. maculans isolates harboring the AvrLm3 avirulence gene, selected from the group comprising L. maculans isolates Lm1033-1, Lm1086-2, Lmu36, Lmu52, Lmu37, Lmu38, Lmu58, Lmu60, Lmu65 and Lmu77-3. In a further embodiment, the Rlm3 blackleg resistance gene as referred to herein confers resistance to a Leptosphaeria maculans strain harboring the AvrLm3 avirulence gene, such as Leptosphaeria maculans Isolate Lm1033-1.

Enhanced resistance can also be assessed in bioassays carried out in controlled environments, such as growth chambers, but ideally are confirmed in field trials, as controlled environment assessments often do not reflect field conditions. This may be due to the fact that few, single spore isolates of the fungus are normally tested in bioassays, while in the field much larger variation in the pathogen population exists.

A Rlm3 blackleg resistance gene, or Rlm3 gene can encode a Rlm3 amino acid sequence having at least 80%, or at least 85%, or at least 90%, or at least 92%, or at least 95%, or at least 98%, or at least 99%, or 100% sequence identity to SEQ ID NO: 43, or to SEQ ID NO: 5, or to SEQ ID NO: 45. A Rlm3 blackleg resistance gene, or Rlm3 gene, can comprise a nucleotide sequence having at least 80%, or at least 85%, or at least 90%, or at least 92%, or at least 95%, or at least 98%, or at least 99%, or 100% sequence identity to SEQ ID NO: 1, SEQ ID NO: 42, or to SEQ ID NO: 4, or to SEQ ID NO: 44. Said Rlm3 blackleg resistance gene, or Rlm3 gene may further comprise one or more introns. Further, it can comprise a nucleotide sequence having at least 80%, or at least 85%, or at least 90%, or at least 92%, or at least 95%, or at least 98%, or at least 99%, or 100% sequence identity to nt 2106 to 9496 of SEQ ID NO: 1, to nt 2106-4688 of SEQ ID NO: 1, or to nt 5908-9496 of SEQ ID NO: 1.

For the purpose of this invention, the “identity” or “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48 (3): 443-53) in The European Molecular Biology Open Software Suite (EMBOSS, Rice et al., 2000, Trends in Genetics 16 (6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)). For nucleotides the default scoring matrix used is EDNA-FULL and for proteins the default scoring matrix is EBLOSUM62. It will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

The term “at least 80%” means 80% or more, such as at least 85%, at least 87% at least 90%, at least 92%, at least 95%, at least 96%, at least 97%, at least 98% at least 99%, or 100%.

The term “at least 90%” means 90% or more, such as at least 95%, at least 96%, at least 97%, at least 98% or at least 99% or 100%.

“Stringent hybridization conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2×SSC at 63° C. for 20 min, or equivalent conditions.

“High stringency conditions” can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency conditions” refers to conditions equivalent to hybridization in the above described solution but at about 60-62° C. Moderate stringency washing may be done at the hybridization temperature in 1×SSC, 0.1% SDS.

“Low stringency” refers to conditions equivalent to hybridization in the above described solution at about 50-52° C. Low stringency washing may be done at the hybridization temperature in 2×SSC, 0.1% SDS. See also Sambrook et al. (1989) and Sambrook and Russell (2001).

A “Rlm3 blackleg resistance locus” as used herein refers to the genetic locus that comprises a Rlm3 blackleg resistance gene. A “Rlm3 blackleg resistance locus” refers to the position on the chromosome where a “Rlm3 blackleg resistance gene” is located. This position can be identified by the location on the genetic map of a chromosome. Included in this definition is the fragment (or segment) of genomic DNA of the chromosome on which the Rlm3 blackleg resistance locus is located. Said Rlm3 blackleg resistance gene can be a native Rlm3 blackleg resistance gene in its native chromosomal position, or can be a transgene on a chromosomal position on which it does not occur naturally.

The Rlm3 blackleg resistance locus can comprise the Rlm3 blackleg resistance genes according to the invention. In particular, the Rlm3 blackleg resistance locus can comprise a sequence having at least 80%, or at least 85%, or at least 90%, or at least 92%, or at least 95%, or at least 98%, or at least 99%, or 100% sequence identity to SEQ ID NO: 1. The sequences may be interrupted by non-translated sequences, such as introns. In an embodiment, the Rlm3 blackleg resistance locus comprises a sequence having at least 80% sequence identity to nt 2106 to 9496 of SEQ ID NO: 1, to nt 2106-4688 of SEQ ID NO: 1, or to nt 5908-9496 of SEQ ID NO: 1.

In a further embodiment, the Rlm3 blackleg resistance locus comprises two polynucleotides:
a) a first polynucleotide having a nucleic acid sequence which is at least 80%, at least 85%, at least 90%, at least 95%, at least 96%, at least 97%, at least 98%, at least 99% or 100% identical to the nucleic acid sequence of SEQ ID NO: 4 or to nt 2106-4688 of SEQ ID NO: 1; and
b) a second polynucleotide having a nucleic acid sequence which is at least 80%, at least 85%, at least 90%, at least 95%, at least 96%, at least 97%, at least 98%, at least 99% or 100% identical to the nucleic acid sequence of SEQ ID NO: 44 or to nt 5908-9496 of SEQ ID NO: 1.

A “locus” as used herein is the position that a gene occupies on a chromosome. A “blackleg resistance locus” refers to the position on the chromosome where a “blackleg resistance gene” is located. This position can be identified by the location on the genetic map of a chromosome. Included in this definition is the fragment (or segment) of genomic DNA of the chromosome on which the blackleg resistance locus is located. Said blackleg resistance gene can be a native blackleg resistance gene in its native chromosomal position, or can be a transgene on a chromosomal position on which it does not occur naturally. Said blackleg resistance gene can be the Rlm3 blackleg resistance gene or another blackleg resistance gene. A locus which does not comprise the Rlm3 blackleg resistance gene according to the invention, which is at the position on the chromosome corresponding to the position where the Rlm3 blackleg resistance gene is located in a resistant line, can be referred to as “Rlm3 blackleg susceptibility locus”.

The polynucleotide comprising an “Rlm3 associated open reading frame” shall encode for a polypeptide of the present invention. The polynucleotide may comprise exons, introns, UTRs, etc. Thus, the polynucleotide may comprise sequences which are not translated such as introns and 5′ and 3′ untranslated regions. According the polynucleotide comprising an “Rlm3 associated open reading frame” shall encode for transcript which is processed to an mRNA (e.g. via splicing) which encodes the polypeptides of the present invention.

The term “introduction” or “introducing” is understood by the skilled person. As used herein, it encompasses any method for introducing a gene into a plant. In an embodiment, the Rlm3 gene is introduced into a plant by crossing two plants. In an embodiment, the Rlm3 polynucleotide and/or Rlm3 protein is introduced into a plant by crossing two plants, whereas one plant comprises the Rlm3 polynucleotide and/or Rlm3 protein of the present invention. The second plant may lack said Rlm3 polynucleotide and/or Rlm3 protein. In an alternative embodiment, the gene is introduced by gene editing. The term is described elsewhere herein. In a third embodiment, the gene is introduced by transformation. The term “transformation” as referred to herein encompasses the transfer of an exogenous polynucleotide into a host cell. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8:363-373); electroporation of protoplasts (Shillito R. D. et al. (1985) Bio/Technol 3, 1099-1102); microinjection into plant material (Crossway A et al., (1986) Mol. Gen Genet 202:179-185); DNA or RNA-coated particle bombardment (Klein T M et al., (1987) Nature 327:70) infection with (non-integrative) viruses and the like. Transgenic plants are preferably produced via Agrobacterium-mediated transformation. The genetically modified plant cells can be regenerated via all methods with which the skilled worker is familiar. After introduction, the plant may be selected for the presence of the Rlm3 polynucleotide and/or Rlm3 protein of the present invention.

As used herein, the term “plant-expressible promoter” means a DNA sequence that is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Harpster et al. (1988) Mol Gen Genet. 212 (1): 182-90, the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al. (1996) Plant Cell 8 (1): 15-30), stem-specific promoters (Keller et al., (1988) EMBO J. 7 (12): 3625-3633), leaf specific promoters (Hudspeth et al. (1989) Plant Mol Biol. 12:579-589), mesophyl-specific promoters (such as the light-inducible Rubisco promoters), root-specific promoters (Keller et al. (1989) Genes Dev. 3:1639-1646), tuber-specific promoters (Keil et al. (1989) EMBO J. 8 (5): 1323-1330), vascular tissue specific promoters (Peleman et al. (1989) Gene 84:359-369), stamen-selective promoters (WO 89/10396, WO 92/13956), dehiscence zone specific promoters (WO 97/13865) and the like.

Suitable promoters for the invention are constitutive plant-expressible promoters. Constitutive plant-expressible promoters are well known in the art, and include the CaMV35S promoter (Harpster et al. (1988) Mol Gen Genet. 212 (1): 182-90), Actin promoters, such as, for example, the promoter from the Rice Actin gene (McElroy et al., 1990, Plant Cell 2:163), the promoter of the Cassava Vein Mosaic Virus (Verdaguer et al., 1996 Plant Mol. Biol. 31:1129), the GOS promoter (de Pater et al., 1992, Plant J. 2:837), the Histone H3 promoter (Chaubet et al., 1986, Plant Mol Biol 6:253), the Agrobacterium tumefaciens Nopaline Synthase (Nos) promoter (Depicker et al., 1982, J. Mol. Appl. Genet. 1:561), or Ubiquitin promoters, such as, for example, the promoter of the maize Ubiquitin-1 gene (Christensen et al., 1992, Plant Mol. Biol. 18:675).

A further promoter suitable for the invention is the endogenous promoter driving expression of the gene encoding an Rlm3 protein.

A “transcription termination and polyadenylation region” as used herein is a sequence that drives the cleavage of the nascent RNA, whereafter a poly(A) tail is added at the resulting RNA 3′ end, functional in plant cells. Transcription termination and polyadenylation signals functional in plant cells include, but are not limited to, 3′nos, 3′35S, 3′his and 3′g7.

The term “plant” as used herein preferably refers to Brassicaceae plant, such as a Brassica plant.

“Brassicaceae” or “Brassicaceae plant” as used herein refers to plants belonging to the family of Brassicaceae plants, also called Cruciferae or mustard family. Examples of Brassicaceae are, but are not limited to, Brassica species, such as Brassica napus, Brassica oleracea, Brassica rapa, Brassica carinata, Brassica nigra, and Brassica juncea; Raphanus species, such as Raphanus caudatus, Raphanus raphanistrum, and Raphanus sativus, Matthila species; Cheiranthus species; Camelina species, such as Camelina sativa; Crambe species, such as Crambe abyssinica and Crambe hispanica; Eruca species, such as Eruca vesicaria, Sinapis species such as Sinapis alba, Diplotaxis species; Lepidium species; Nasturtium species; Orychophragmus species; Armoracia species, Eutrema species; Lepidium species; and Arabidopsis species.

A “Brassica plant” refers to allotetraploid or amphidiploid Brassica napus (AACC, 2n=38), Brassica juncea (AABB, 2n=36), Brassica carinata (BBCC, 2n=34), or to diploid Brassica rapa (syn. B. campestris) (AA, 2n=20), Brassica oleracea (CC, 2n=18) or Brassica nigra (BB, 2n=16).

In one embodiment, the plant is Brassica napus. For example, the plant may be Brassica napus cultivar (cv) Westar or Brassica napus cultivar Darmor. Both cultivars are susceptible to blackleg (see Examples). In another embodiment, the plant is Brassica oleracea. In another embodiment, the plant is Brassica rapa. In another embodiment, the plant is Brassica nigra. In another embodiment, the plant is Brassica juncea. In another embodiment, the plant is Brassica carinata.

As set forth herein above, the identified Rlm3 proteins show homology to WAKL10 (Wall-associated receptor kinase-like 10) proteins. WALK10 proteins are serine/threonine-protein kinase that may function as a signaling receptor of extracellular matrix component. Accordingly, a Rlm3 protein as set forth herein, such as the protein comprising an amino acid sequence as shown in SEQ ID NO: 43, SEQ ID NO: 5, or SEQ ID NO: 45 (or variants thereof) may have kinase activity. In particular, it may have serine/threonine-protein kinase activity. A serine/threonine protein kinase (EC 2.7.11.−) is a kinase enzyme that phosphorylates the OH group of serine or threonine.

Without being bound to theory, it is believed that the Rlm3 polypeptide of the invention acts as a receptor which recognizes the blackleg pathogen, and activates the resistance mechanism through its kinase activity.

It is another object of the invention to provide a method for increasing blackleg resistance in a Brassicaceae plant, said method comprising introducing or providing a Rlm3 blackleg resistance gene according to the invention as a transgene to a Brassicaceae plant cell, to create transgenic cells; and regenerating transgenic plants from said transgenic cells.

A transgene can be provided to a plant or plant cell using methods well-known in the art. Methods for introduction of genes into plant cells to create transgenic plants are not deemed critical for the current invention and any method to provide plant cells with a transgene suitable for a particular plant species can be used. Such methods are well known in the art and include Agrobacterium-mediated transformation, particle gun delivery, microinjection, electroporation of intact cells, polyethyleneglycol-mediated protoplast transformation, electroporation of protoplasts, liposome-mediated transformation, silicon-whiskers mediated transformation etc. Said transgene may be stably integrated into the genome of said plant cell, resulting in a transformed plant cell. The transformed plant cells obtained in this way may then be regenerated into mature fertile transformed plants.

In an embodiment of the present invention, the Rlm3 resistance gene of the present invention is introduced into a plant using genome editing. Accordingly, a method is provided for producing a blackleg resistant plant (such as a Brassica plant) comprising introducing the Rlm3 resistance gene into a plant not comprising a functional Rlm3 resistance gene using genome editing.

In another embodiment, a blackleg resistant plant is produced by introducing into the genome of that plant two polynucleotides comprising a Rlm3 associated open reading frame. A first polynucleotide comprising a Rlm3 associated open reading frame encoding a polypeptide comprising an amino acid sequence is selected from the group comprising:
a) an amino acid sequence which is at least 80%, at least 85%, at least 90%, at least 95%, at least 96%, at least 97%, at least 98%, at least 99% or at least 100% identical to the amino acid sequence shown in SEQ ID NOs: 5;
b) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NO: 4;
c) a partial sequence of a) or b).

In addition, a second polynucleotide comprising a Rlm3 associated open reading frame encoding a polypeptide is introduced into the genome of that plant comprising an amino acid sequence selected from the group comprising:
a) an amino acid sequence which is at least 80%, at least 85%, at least 90%, at least 95%, at least 96%, at least 97%, at least 98%, at least 99% or at least 100% identical to the amino acid sequence shown in SEQ ID NOs: 45;
b) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NO: 44;
c) a partial sequence of a) or b).

In an embodiment, the plant not comprising a functional Rlm3 resistance gene is a plant comprising a silent allele of a gene encoding a polypeptide comprising a Rlm3 associated open reading frame.

The present invention method of conferring blackleg resistance to a plant comprising the step of genetically modifying a silent allele of a gene encoding a polypeptide comprising a Rlm3 associated open reading frame such that the silent allele is capable of expressing said polypeptide, wherein said Rlm3 associated open reading frame encodes a polypeptide comprising an amino acid sequence selected from the group consisting of:
a) an amino acid sequence as shown in any one of SEQ ID NOs: 43, 5 and 45;
b) an amino acid sequence which is at least 80%, at least 85%, at least 90%, at least 95%, at least 96%, at least 97%, at least 98% or at least 99% identical to the amino acid se-quence shown in any one of SEQ ID NOs: 43, 5 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 42, 4 and 44, wherein said polypeptide is capable of conferring blackleg resistance to a plant;
d) a partial sequence of any one of a) to c), wherein said polypeptide is capable of conferring blackleg resistance to a plant.

In an embodiment of the aforementioned method, the silent allele is modified by homologous recombination or by genome editing technology.

The term “silent allele” as used herein refers to an allele which does not confer blackleg resistance. A silent allele can differ from the Rlm3 allele in that the gene is absent, the gene is not expressed, or the gene encodes a different protein.

Genome editing, also called gene editing, genome engineering, as used herein, refers to the targeted modification of genomic DNA in which the DNA may be inserted, deleted, modified or replaced in the genome. Genome editing may use sequence-specific enzymes (such as endonuclease, nickases, base conversion enzymes) and/or donor nucleic acids (e.g. dsDNA, oligo's) to introduce desired changes in the DNA. Sequence-specific nucleases that can be programmed to recognize specific DNA sequences include meganucleases (MGNs), zinc-finger nucleases (ZFNs), TAL-effector nucleases (TALENs) and RNA-guided or DNA-guided nucleases such as Cas9, Cpf1, CasX, CasY, C2cl, C2c3, certain Argonaut-based systems (see e.g. Osakabe and Osakabe, Plant Cell Physiol. 2015 March; 56 (3): 389-400; Ma et ah, Mol Plant. 2016 Jul. 6; 9 (7): 961-74; Bortesie et al, Plant Biotech J, 2016, 14; Murovec et ah, Plant Biotechnol J. 15:917-926, 2017; Nakade et ah, Bioengineered Vol 8, No. 3:265-273, 2017; Burstein et ah, Nature 542, 37-241; Komor et ah, Nature 533, 420-424, 2016; all incorporated herein by reference). Donor nucleic acids can be used as a template for repair of the DNA break induced by a sequence specific nuclease. Donor nucleic acids can also be used as such for genome editing without DNA break induction to introduce a desired change into the genomic DNA.

In yet another embodiment, a method is provided for increasing blackleg resistance in a Brassicaceae plant, comprising the step of introducing a Rlm3 blackleg resistance locus in said Brassicaceae plant, and selecting said blackleg resistant Brassicaceae plant for the presence of the Rlm3 blackleg resistance locus by analyzing genomic DNA from said plant for the presence of at least one molecular marker, wherein said at least one molecular marker is linked to the Rlm3 blackleg resistance locus, wherein said Rlm3 blackleg resistance locus comprises the Rlm3 blackleg resistance gene, wherein said Rlm3 blackleg resistance gene comprises a nucleotide sequence having at least 80% sequence identity to SEQ ID NO: 1, or to nt 2106 to 9496, or to nt 2106-4688, or to nt 5908-9496 of SEQ ID NO: 1; a coding sequence having at least 80% sequence identity to SEQ ID NO: 42, SEQ ID NO: 4, or to SEQ ID NO: 44; or encoding a protein having at least 80% sequence identity to SEQ ID NO: 43, SEQ ID NO: 5, or SEQ ID NO: 45, such as a Rlm3 blackleg resistance locus comprising a sequence having at least 80% sequence identity to SEQ ID NO: 1. Said method can comprise the provision of a first Brassicaceae plant comprising a Rlm3 blackleg resistance locus, providing a second Brassicaceae plant lacking a Rlm3 blackleg resistance locus, crossing the first Brassicaceae plant with the second Brassicaceae plant to provide progeny Brassicaceae plant; analyzing said progeny Brassicaceae plant to determine the presence of a Rlm3 blackleg resistance locus by analyzing genomic DNA from the plant for the presence of at least one molecular marker, wherein said at least one molecular marker is linked to the Rlm3 blackleg resistance locus; and selecting Brassicaceae progeny that tests positive for the presence of the Rlm3 blackleg resistance locus as being Brassicaceae plant into which the Rlm3 blackleg resistance locus has been introgressed. Said first Brassicaceae plant may be obtained by screening a population of Brassicaceae plants for the presence of a Rlm3 blackleg resistance locus by analyzing genomic DNA from the plant for at least one molecular marker, wherein said at least one molecular marker is linked to the Rlm3 blackleg resistance locus. Said first Brassicaceae plant and said progeny Brassicaceae plant may be Brassica rapa; and first Brassicaceae plant and said progeny Brassicaceae plant may be Brassica napus, or said first Brassicaceae plant may be Brassica rapa, said second Brassicaceae plant may be Brassica oleracea, and said progeny Brassicaceae plant may be Brassica napus obtained through an interspecific cross between said first and said second Brassicaceae plant.

Yet another embodiment provides a method for producing a blackleg resistant Brassicaceae plant comprising the steps of identifying a blackleg resistant Brassicaceae plant comprising a Rlm3 blackleg resistance locus according to the invention by analyzing genomic DNA from said plant for the presence of at least one molecular marker, wherein said at least one molecular marker is linked to said Rlm3 blackleg resistance locus, and generating progeny from said blackleg resistant Brassicaceae plant, wherein said progeny is blackleg resistant and comprises said Rlm3 blackleg resistance locus. In a further embodiment, said Rlm3 blackleg resistance locus comprises a sequence having at least 80% sequence identity to SEQ ID NO: 1. Alternatively, said Rlm3 blackleg resistance locus comprises a sequence having at least 80% sequence identity to 2106 to 9496, or to nt 2106-4688, or to nt 5908-9496 of SEQ ID NO: 1.

A “molecular marker”, or a “marker”, as used herein, refers to a polymorphic locus, i.e. a polymorphic nucleotide (a so-called single nucleotide polymorphism or SNP) or a polymorphic DNA sequence (which can be insertion of deletion of a specific DNA sequence at a specific locus, or polymorphic DNA sequences). A marker refers to a measurable, genetic characteristic with a fixed position in the genome, which is normally inherited in a Mendelian fashion, and which can be used for mapping of a trait of interest. Thus, a molecular marker may be a short DNA sequence, such as a sequence surrounding a single base-pair change, i.e. a single nucleotide polymorphism or SNP, or a long DNA sequence, such as microsatellites or Simple Sequence Repeats (SSRs). The nature of the marker is dependent on the molecular analysis used and can be detected at the DNA, RNA or protein level. Genetic mapping can be performed using molecular markers such as, but not limited to, RFLP (restriction fragment length polymorphisms; Botstein et al. (1980), Am J Hum Genet 32:314-331; Tanksley et al. (1989), Bio/Technology 7:257-263), RAPD [random amplified polymorphic DNA; Williams et al. (1990), NAR 18:6531-6535], AFLP [Amplified Fragment Length Polymorphism; Vos et al. (1995) NAR 23:4407-4414]. Appropriate primers or probes are dictated by the mapping method used.

The term “marker allele” refers to the version of the marker that is present in a particular plant at one of the chromosomes. Typically, a marker can exist as or can be said to have or to comprise two marker alleles. The term “haplotype”, as used herein, refers to a specific combination of marker alleles as present within a certain plant or group of (related) plants. As described herein, a marker allele can be the version of the marker that is present in the resistant line (Rlm3 blackleg resistance marker allele). The version of the same marker that is present in the susceptible line can be referred to as Rlm3 blackleg susceptibility marker allele.

The term “AFLP?” (AFLP& is a registered trademark of KeyGene N.V., Wageningen, The Netherlands), “AFLP analysis” and “AFLP marker” is used according to standard terminology (Vos et al. (1995), NAR 23:4407-4414; EP0534858). Briefly, AFLP analysis is a DNA fingerprinting technique which detects multiple DNA restriction fragments by means of PCR amplification. The AFLP technology usually comprises the following steps: (i) the restriction of the DNA with two restriction enzymes, preferably a hexa-cutter and a tetra-cutter, such as EcoRI, PstI and MseI; (ii) the ligation of double-stranded adapters to the ends of the restriction fragments, such as EcoRI, PstI and MseI adaptors; (iii) the amplification of a sub-set of the restriction fragments using two primers complementary to the adapter and restriction site sequences, and extended at their 3′ ends by one to three “selective” nucleotides, i.e., the selective amplification is achieved by the use of primers that extend into the restriction fragments, amplifying only those fragments in which the primer extensions match the nucleotides flanking the restriction sites. AFLP primers thus have a specific sequence and each AFLP primer has a specific code; (iv) gel electrophoresis of the amplified restriction fragments on denaturing slab gels or capillaries; (v) the visualization of the DNA fingerprints by means of autoradiography, phosphor-imaging, or other methods. Using this method, sets of restriction fragments may be visualized by PCR without knowledge of nucleotide sequence. An AFLP marker, as used herein, is a DNA fragment of a specific size, which is generated and visualized as a band on a gel by carrying out an AFLP analysis. Each AFLP marker is designated by the primer combination used to amplify it, followed by the approximate size (in base pairs) of the amplified DNA fragment. It is understood that the size of these fragments may vary slightly depending on laboratory conditions and equipment used. Every time reference is made herein to an AFLP marker by referring to a primer combination and the specific size of a fragment, it is to be understood that such size is approximate, and comprises or is intended to include the slight variations observed in different labs. Each AFLP marker represents a certain locus in the genome.

The term “SSR” refers to Simple Sequence Repeats or microsatellite [Tautz et al. (1989), NAR 17:6463-6471]. Short Simple Sequence stretches occur as highly repetitive elements in all eukaryotic genomes. Simple sequence loci usually show extensive length polymorphisms. These simple sequence length polymorphisms (SSLP) can be detected by polymerase chain reaction (PCR) analysis and be used for identity testing, population studies, linkage analysis and genome mapping.

It is understood that molecular markers can be converted into other types of molecular markers. When referring to a specific molecular marker in the present invention, it is under-stood that the definition encompasses other types of molecular markers used to detect the genetic variation originally identified by the specific molecular markers. For example, if an AFLP marker is converted into another molecular marker using known methods, this other marker is included in the definition. For example, AFLP markers can be converted into sequence-specific markers such as, but not limited to STS (sequenced-tagged-site) or SCAR (sequence-characterized-amplified-region) markers using standard technology as described in Meksem et al. [(2001), Mol Gen Genomics 265 (2): 207-214], Negi et al. [(2000), TAG 101:146-152], Barret et al. (1989), TAG 97:828-833], Xu et al. [(2001), Genome 44 (1): 63-70], Dussel et al. [(2002), TAG 105:1190-1195] or Guo et al. [(2003), TAG 103:1011-1017]. For example, Dussel et al. [(2002), TAG 105:1190-1195] converted AFLP markers linked to resistance into PCR-based sequence tagged site markers such as indel (insertion/deletion) markers and CAPS (cleaved amplified polymorphic sequence) markers.

Suitable molecular markers are, for example SNP markers (Single Nucleotide Polymorphisms), AFLP markers, microsatellites, minisatellites, Random Amplified Polymorphic DNA's (RAPD) markers, RFLP markers, Sequence Characterized Amplified Regions (SCAR) markers, and others, such as TRAP markers described by Hu et al. 2007, Genet Resour Crop Evol 54:1667-1674).

Methods and assays for marker detection, or for analyzing the genomic DNA for the presence of a marker, are widely known in the art. The presence of a marker can, for example be detected in hybridization-based methods (e.g. allele-specific hybridization), using Taqman, Invader, PCR-based methods, oligonucleotide ligation based methods, or sequencing-based methods.

A useful assay for detection of SNP markers is for example KBioscience Competitive Allele-Specific PCR. For developing the KASP-assay 70 base pairs upstream and 70 base-pairs downstream of the SNP are selected and two allele-specific forward primers and one allele specific reverse primer is designed. See e.g. Allen et al. 2011, Plant Biotechnology J. 9, 1086-1099, especially p 1097-1098 for KASP assay method (incorporated herein by reference).

A “molecular marker linked to the Rlm3 blackleg resistance locus”, or a “molecular marker linked to the presence of the Rlm3 blackleg resistance locus” as used herein refers to a molecular marker in a region in the genome that inherits with the Rlm3 blackleg resistance locus as a single genetic unit in at least 50% of the cases. Thus, in this respect, the term linked can be a separation of about 50 cM, or less such as about 40 cM, about 30 CM, about 20 cM, about 10 cM, about 7.5 CM, about 6 cM, about 5 CM, about 4 cM, about 3 cM, about 2.5 CM, about 2 cM, or even less.

A “molecular marker linked to the Rlm3 blackleg resistance locus”, or a “molecular marker linked to the presence of the Rlm3 blackleg resistance locus” can also be a marker located in the sequence shown in SEQ ID NO: 1, for example in the region of nt 2106 to nt 9496 of SEQ ID NO: 1, or in the region of nt 2106-4688, or nt 5908-9496 of SEQ ID NO: 1.

Suitable are markers that are linked to the Rlm3 blackleg resistance locus can be developed using methods known in the art. New markers suitable for the invention can be developed based on the Rlm3 sequence. It is understood that such markers can be developed by com-paring the sequence of the Rlm3 blackleg resistance locus from the resistant Brassicaceae line with the sequence of the same locus in a susceptible Brassicaceae line; identifying a specific sequence region in the Rlm3 blackleg resistance locus which does not occur in the corresponding locus of the susceptible Brassicaceae line. A molecular marker linked to the Rlm3 blackleg resistance locus can thus be a marker detecting the presence of the Rlm3 blackleg resistance locus, or can be a marker directly detecting the presence of the se-quence of SEQ ID NO: 1. A molecular marker linked to the Rlm3 blackleg resistance locus can also be a marker in the sequences flanking the Rlm3 blackleg resistance locus, which is polymorphic between lines comprising the Rlm3 blackleg resistance locus and lines lacking, but which inherits with the Rlm3 blackleg resistance locus as a single genetic unit in at least 50% of the cases.

Markers suitable to determine the presence of the Rlm3 blackleg resistance locus can be the markers that are linked to Rlm3 blackleg resistance locus.

The absence of the Rlm3 blackleg resistance locus can be determined by the absence of marker alleles that are linked to the presence of the Rlm3 blackleg resistance locus (Rlm3 blackleg resistance marker alleles). Furthermore, markers suitable to determine the absence of the Rlm3 blackleg resistance locus can be marker alleles which are linked to the Rlm3 blackleg susceptibility locus (Rlm3 blackleg susceptibility marker alleles).

Analysis for the presence of markers according to the invention can be performed with a first primer and a second primer, and, optionally, a probe, selected from the group of oligo-nucleotides consisting of a first primer consisting of a sequence of 15 to 30 nucleotides, or 15 to 25 nucleotides, or 18 to 22 nucleotides of the Rlm3 blackleg resistance genes according to the invention, a second primer being complementary to a sequence of 15 to 30 nucleotides, or 15 to 25 nucleotides, or 18 to 22 nucleotides of the Rlm3 blackleg resistance genes according to the invention, and wherein the distance between said first and said second primer on the Rlm3 blackleg resistance gene is between 1 and 400 bases, or between 1 and 150 bases, and wherein the first primer is located, with respect to the Rlm3 coding sequence, upstream of said second primer, and a probe which is identical to at least 15 nucleotides, or at least 18 nucleotides, but not more than 25 nucleotides, or not more than 22 nucleotides of the sequence of the Rlm3 blackleg resistance gene between said first and said second primer, provided that either the sequence of the first primer, or the sequence of the second primer, or the sequence of said probe is not present in the corresponding locus in a susceptible Brassicaceae plant. Said probe may be labelled, such as, for example, described in U.S. Pat. No. 5,538,848.

Analysis for the presence of markers according to the invention can be performed with a first and second primer as described above recognizing both the Rlm3 sequence and the corresponding locus in the susceptible Brassicaceae line, a first probe recognizing a sequence of the Rlm3 blackleg resistance gene as described above, but not recognizing a sequence between said first and said second primer in the susceptible Brassicaceae line, and a second probe recognizing a sequence between said first and said second primer in the susceptible Brassicaceae line, but not of the Rlm3 blackleg resistance gene, and wherein said the label of the first probe is different from that of the second probe.

Further suitable primers for analysis of the presence of markers according to the invention are a first primer recognizing both the Rlm3 sequence and the corresponding locus in the susceptible Brassicaceae line, a second primer recognizing the Rlm3 sequence but not the corresponding locus in the susceptible Brassicaceae line, and a third primer recognizing the corresponding locus in the susceptible Brassicaceae line but not the Rlm3 sequence. Said second and third primer may be labelled as indicated above, and said second primer may contain a label which is different from said third primer.

Identification of PCR products specific for the Rlm3 blackleg resistance genes and for the corresponding locus in the susceptible Brassicaceae line can occur e.g. by size estimation after gel or capillary electrophoresis (e.g. for the Rlm3 blackleg resistance locus and for the corresponding locus in the susceptible Brassicaceae line comprising a number of inserted or deleted nucleotides which results in a size difference between the fragments amplified from the Rlm3 blackleg resistance locus and for the corresponding locus in the susceptible Brassicaceae, such that said fragments can be visibly separated on a gel); by evaluating the presence or absence of the two different fragments after gel or capillary electrophoresis, whereby the diagnostic PCR amplification of the Rlm3 blackleg resistance locus can, optionally, be performed separately from the diagnostic PCR amplification of the corresponding locus in the susceptible line; by direct sequencing of the amplified fragments; or by fluorescence-based detection methods.

A further embodiment provides Brassicaceae plants or plant cells obtainable by the methods according to the invention, such as Brassica napus, Brassica juncea, Brassica oleracea, Brassica rapa, Brassica nigra or Brassica carinata plants.

In a further embodiment, a blackleg resistant Brassicaceae plant or plant cell according to the invention is provided, comprising the Rlm3 blackleg resistance gene according to the invention, and at least one other disease resistance gene, said other disease resistance gene selected from the group consisting of a clubroot resistance gene, a further blackleg resistance gene, a Sclerotinia resistance gene, a Verticillium resistance gene, a Fusarium resistance gene, an Aster Yellows resistance gene, an Alternaria resistance gene, and a Grey Stem resistance gene. In a further embodiment, said other disease resistance gene is a transgene which is genetically linked with said Rlm3 blackleg resistance gene of the present invention.

Said clubroot resistance gene may be a Crr2, Crr4, Crr3, CRk, CRc, CR2a, CR2b, pb-3, pb-4, Pb-Bo1, Pb-Bo2, Pb-Bo3, Pb-Bo4, Pb-Bo5a, Pb-Bo5b, Pb-Bo8, Pb-Bo9a, Pb-Bo9b, Pb-Bn1, PbBn-01:60-1, PbBn-01:60-2, PbBn-01:60-3, PbBn-01:60-4, PbBn-01:07-1, PbBn-01:07-2, PbBn-01:07-3, PbBn-e4x04-1, PbBn-a-1, PbBn-1-1, PbBn-1-2, PbBn-k-1, PbBn-k-2. PbBn-k-3, PbBn-Korp-1, PbBn-Korp-2, PbBn-Korp-3, PbBn-Korp-4, PbBn-Korp-5 as described by Piao (Piao et al., 2009, J Plant Growth Regul 28:252), or may be a CRa gene as described by Ueno et al. (Ueno et al., 2012, Plant Mol Biol 80:621), a Crr1 gene as described by Hatakeyama et al., (Hatakeyama et al., 2013, PLOS one 8: e54745) and in WO2012/039445, or a CRb gene as described by Kato et al., (Kato et al., 2013, Breeding Science 63:116), (herein incorporated by reference). Moreover, said clubroot resistance gene may be a CLR1 or CLR2 gene as described in WO 2017/102923 A1 (herein incorporated by reference).

Said further blackleg resistance gene may, for example, be BLMR1 and BLMR2 (WO 2011/044694), LepR3 (Larkan et al., 2013, New Phytol 197:595 and WO 2008/101343), or Lem-08-syl (EP 1547462 and US 2005/0142122). Moreover, said further blackleg resistance gene may be Rlm1, Rlm2, Rlm4, Rlm5, Rlm6, Rlm7, Rlm8, Rlm9, Rlm10, Rlm11, RlmJ1, RlmS, LepR1, LepR2, LepR4, LmJR1 or LmJR2 (Larkan et al., 2016, supra).

In an embodiment, a blackleg resistant Brassicaceae plant or plant cell according to the invention is provided, comprising the Rlm3 blackleg resistance gene according to the invention, and at least one additional blackleg resistance gene selected from the group consisting of Rlm4, Rlm7 and Rlm9. Introduction of additional blackleg resistance genes in a single plant or plant cell is especially advantageous as R genes have been reported to convey race-specific resistance against L. maculans due to their interaction with specific L. maculans avirulence genes. As used herein, Rlm4 gene refers to a polynucleotide encoding a polypeptide comprising an amino acid sequence which is at least 80%, at least 85%, at least 90%, at least 95%, at least 96%, at least 97%, at least 98%, at least 99% or 100% identical to the amino acid sequence shown in any one of SEQ ID NO: 32. As used herein, Rlm7 gene refers to a polynucleotide encoding a polypeptide comprising an amino acid sequence which is at least 80%, at least 85%, at least 90%, at least 95%, at least 96%, at least 97%, at least 98%, at least 99% or 100% identical to the amino acid sequence shown in any one of SEQ ID NO: 35. As used herein, Rlm9 gene refers to a polynucleotide encoding a polypeptide comprising an amino acid sequence which is at least 80%, at least 85%, at least 90%, at least 95%, at least 96%, at least 97%, at least 98%, at least 99% or 100% identical to the amino acid sequence shown in any one of SEQ ID NO: 38.

A Sclerotinia resistance gene may be a sclerotinia resistance gene as described in WO 2005/090578.

Said other disease resistance gene may be present in their native chromosomal position. For example, said other disease resistance genes can be introduced by introgression in the plant according to the invention from the cultivar or—species from which they are derived.

The Rlm3 blackleg resistance gene according to the invention may be transferred to another position in the plant genome in which it is genetically linked to another trait of interest, such as another disease resistance gene as described herein.

It is understood that environmental conditions, such as location, weather conditions and disease pressure, as well as individual perception of the person assessing disease symptoms, can have an effect on the scoring of blackleg resistance. Hence, variation in these factors in comparative tests should be minimized. Any other resistance ratings known in the art can be applied in accordance with this invention to compare the plants of the invention with control plants.

How to assess, whether a plant is resistant (or susceptible) is well known in the art. Further, it is described in the Examples section, see Example 2. For example, a scoring system can be applied using a scale from 1 to 9, with 1 being susceptible and 9 being resistant. Further, a scoring system is disclosed in Khangura et al. (2003, Department of Agriculture, Western Australia, Farmnote No. 6/2003, ISSN 0726-934 x).

In a further embodiment, said other disease resistance gene is a transgene which is genetically linked with the blackleg resistance gene. A transgene, as used herein, refers to a gene which is stably integrated in the plant cell at another position than where it occurs naturally. A transgene can, for example, be integrated into the genome of a plant cell, or it can be present on an artificial chromosome. A transgene can, for example, be a gene introduced into a plant species or cultivar in which it does not occur naturally, or it can be a gene introduced in a plant species or cultivar in which it does occur naturally, but at another chromosomal position. A transgene may, but does not need to be a chimeric gene. A transgene may, for example, comprise an expression cassette comprising a coding sequence linked to its endogenous promoter. A transgene may also, for example, comprise a coding sequence linked to a heterologous promoter. Thus, the blackleg resistance gene may be operably linked to said heterologous promoter.

The term “heterologous” polynucleotide, as used herein refers to a polynucleotide that is not native to the host cell. The term “heterologous” polynucleotide also refers to a polynucleotide native to the host cell but with structural modifications (e.g. deletions, substitutions, and/or insertions) as a result of manipulation of the DNA of the host cell by recombinant DNA techniques to alter the native polynucleotide. “Heterologous” polynucleotide also refers to a polynucleotide native to the host cell whose expression is quantitatively altered as a result of manipulation of the regulatory elements of the polynucleotide by recombinant DNA techniques (e.g. a stronger promoter) as well as to a polynucleotide native to the host cell, but integrated not within its natural genetic environment as a result of genetic manipulation by recombinant DNA techniques. With respect to two or more polynucleotide sequences or two or more amino acid sequences, the term “heterologous” is used to characterize that the two or more polynucleotide sequences or two or more amino acid sequences do not occur naturally in the specific combination with each other.

The terms “genetically linked”, “linked”, “linked to” or “linkage”, as used herein, refers to a measurable probability that genes or markers located on a given chromosome are being passed on together to individuals in the next generation. Thus, the term “linked” may refer to one or more genes or markers that are passed together with a gene with a probability greater than 0.5 (which is expected from independent assortment where markers/genes are located on different chromosomes). Because the proximity of two genes or markers on a chromosome is directly related to the probability that the genes or markers will be passed together to individuals in the next generation, the term genetically linked may also refer herein to one or more genes or markers that are located within about 50 centimorgan (cM) or less of one another on the same chromosome. Genetic linkage is usually expressed in terms of cM. Centimorgan is a unit of recombinant frequency for measuring genetic linkage, defined as that distance between genes or markers for which one product of meiosis in 100 is recombinant, or in other words, the centimorgan is equal to a 1% chance that a marker at one genetic locus on a chromosome will be separated from a marker at a second locus due to crossing over in a single generation. It is often used to infer distance along a chromosome. The number of base-pairs to which cM correspond varies widely across the genome (different regions of a chromosome have different propensities towards crossover) and the species (i.e. the total size of the genome). Thus, in this respect, the term linked can be a separation of about 50 cM, or less such as about 40 cM, about 30 cM, about 20 cM, about 10 cM, about 7.5 CM, about 6 cM, about 5 CM, about 4 cM, about 3 cM, about 2.5 CM, about 2 cM, or even less.

The Rlm3 blackleg resistance gene and the other disease resistance gene can be genetically linked when they are stacked as transgenes. For example, Rlm3 blackleg resistance gene and the other disease resistance gene can be present on one construct that is used for transformation. Alternatively, the Rlm3 blackleg resistance gene can be transformed in a Brassica species comprising the other disease resistance gene. The other disease resistance gene can be present either in its native chromosomal context or as a transgene. Directed genome engineering techniques are, for example, based on homologous recombination, or double-strand break induced targeted integration, or site-specific recombination, such as described in, for example, WO2011/154158 or WO2011/154159.

Alternatively, the other disease resistance gene can be transformed in a Brassica species comprising the Rlm3 blackleg resistance gene, provided that it is integrated in the close proximity of the Rlm3 blackleg resistance gene, using directed genome engineering techniques. In the latter case, the other Rlm3 blackleg resistance gene can be present either in its native chromosomal context or as a transgene. The Rlm3 blackleg resistance gene and the other disease resistance gene may also be present on an artificial chromosome. “Artificial chromosomes”, as used herein are constructs that contain DNA sequences and that perform the critical functions of natural chromosomes that allow them to exist independent (autonomously) from native chromosomes. Autonomy during cell division (mitosis) and gamete formation (meiosis) follows from own functional origins of replication and own functional centromere.

In yet another embodiment, the plant according to the invention is selected from the group consisting of Brassica napus, Brassica juncea, Brassica oleracea, Brassica rapa, Brassica nigra and Brassica carinata. In yet another embodiment, seeds, such as hybrid seeds of the plants according to the invention comprising the Rlm3 blackleg resistance gene of the present invention are provided.

Hybrid seeds of the plants according to the invention may be generated by crossing two inbred parental lines, wherein one of the inbred parental lines comprises the Rlm3 blackleg resistance genes according to the invention. In order to produce pure hybrid seeds one of the parental lines is male sterile and is pollinated with pollen of the other line. By growing parental lines in rows and only harvesting the F1 seed of the male sterile parent, pure hybrid seeds are produced. To generate male sterile parental lines, the system as described in EP 0,344,029 or U.S. Pat. No. 6,509,516 may be used, wherein a gene encoding a phytotoxic protein (barnase) is expressed under the control of a tapetum specific promoter, such as TA29, ensuring selective destruction of tapetum cells. Transformation of plants with the chimeric gene pTA29: barnase results in plants in which pollen formation is completely prevented/Mariani et al. (1990), Nature 347:737-741). Cytochemical and histochemical analysis of anther development of Brassica napus plants comprising the chimeric pTA29-barnase gene is described by De Block and De Brouwer ((1993), Planta 189:218-225). To restore fertility in the progeny of a male-sterile plant the male-sterile plant (MS parent) is crossed with a transgenic plant (RF parent) carrying a fertility-restorer gene, which when expressed is capable of inhibiting or preventing the activity of the male-sterility gene (U.S. Pat. Nos. 5,689,041; 5,792,929; De Block and De Brouwer, supra). The use of co-regulating genes in the production of male-sterile plants to increase the frequency of transformants having good agronomical performance is described in WO96/26283. Typically, when the sterility DNA encodes a barnase, the co-regulating DNA will encode a barstar, preferably an optimized barstar gene is used as described in published PCT patent application WO 98/10081. It is understood that different promoters may be used to drive barnase expression in order to render the plant male sterile. Likewise, barstar may be operably linked to different promoters, such as 35S from Cauliflower mosaic virus.

Male sterile plants can also be generated using other techniques, such as cytoplasmic male sterility/restorer systems [e.g. the Ogura system, published U.S. patent application 20020032916, U.S. Pat. No. 6,229,072, WO97/02737, U.S. Pat. No. 5,789,566 or the Polima system of U.S. Pat. No. 6,365,798, WO98/54340 or the Kosena system of WO95/09910, U.S. Pat. No. 5,644,066].

Either the MS parent or the RF parent, or both, may comprise the Rlm3 blackleg resistance gene according to the invention. This can be accomplished by either introducing the Rlm3 blackleg resistance genes into an elite B. napus line and then transforming this line with pTA29-barnase or with pNOS-barstar using known methods. Alternatively the Rlm3 blackleg resistance genes can be introduced directly into a transgenic MS or RF parent line, by crossing a plant comprising the Rlm3 blackleg resistance gene with the MS parent or RF-parent, or by transformation of the MS parent or the RF parent. The F1 hybrid seeds generated from the cross between the MS and RF parent will then contain the Rlm3 blackleg resistance gene.

A further embodiment provides methods to determine the presence or absence of a Rlm3 blackleg resistance locus in a biological sample, comprising providing genomic DNA from said biological sample, and analyzing said DNA for the presence of at least one molecular marker, wherein the at least one molecular marker is linked to the presence or absence of the Rlm3 blackleg resistance locus, wherein said Rlm3 blackleg resistance locus comprises the Rlm3 blackleg resistance gene, wherein said Rlm3 blackleg resistance gene comprises a nucleotide sequence having at least 80% sequence identity to SEQ ID NO: 1, or to nt 2106 to 9496, or to nt 2106-4688, or to nt 5908-9496 of SEQ ID NO: 1; or having a coding sequence having at least 80% sequence identity to SEQ ID NO: 42, SEQ ID NO: 4, or to SEQ ID NO: 44; or encoding a protein having at least 80% sequence identity to SEQ ID NO: 43, SEQ ID NO: 5, or SEQ ID NO: 45.

Moreover, the present invention relates to a method to determine the presence or absence of a Rlm3 polynucleotide of the present invention in a biological sample (such as a plant tissue sample), comprising providing DNA from said biological sample, and analyzing said DNA for the presence or absence of said Rlm3 polynucleotide. The DNA might be genomic DNA. Alternatively, it might be DNA generated by reverse transcription of RNA. The presence or absence of the Rlm3 polynucleotide (blackleg resistance locus Rlm3) can be analyzed as described elsewhere herein. For example, it can be analyzed by using at least one primer or probe which specifically recognizes said polynucleotide. Further, it can be analyzed by using two primers which allow for amplifying at least a portion of said polynucleotide, such as at least 50 bp, at least 100 bp or at least 500 bp.

Moreover, the present invention relates to a method to determine the presence or absence of a Rlm3 polypeptide of the present invention in a biological sample (such as a plant tissue sample), comprising providing polypeptides from said biological sample, and analyzing said polypeptides for the presence or absence of said Rlm3 polypeptide. The presence or absence of the Rlm3 polypeptide can be analyzed by well known methods. For example, it can be analyzed by using at least one antibody which specifically recognizes said Rlm3 polypeptide. Further, it can be analyzed using liquid chromatography-mass spectrometry (LC-MS), in particular liquid chromatography-tandem mass spectrometry (LC-MS/MS). LC-MS and LC-MS/MS may require certain pretreatments of the sample, such as cleaving the polypeptides comprised by the sample with a protease (such as trypsin). By analyzing the sample for Rlm3 polypeptide specific peptides, the presence or absence of said polypeptide can be assessed.

In yet a further embodiment, a kit is provided for the detection of the Rlm3 blackleg resistance locus according to the invention in Brassicaceae DNA samples, wherein said kit comprises at least one primer or probe which specifically recognizes a molecular marker linked to said Rlm3 blackleg resistance locus.

Preferably, the kit for assessing blackleg resistance in a plant comprises the oligonucleotide of the present invention or the antibody of the present invention.

Yet another embodiment provides the use of a molecular marker linked to the Rlm3 blackleg resistance locus according to the invention for determining the presence or absence of said Rlm3 blackleg resistance locus in Brassicaceae plants, or the use of the sequence of any one of SEQ ID NOs: 1, 4, 42, 44 and 46 for determining the presence or absence of the Rlm3 blackleg resistance locus according to the invention in Brassicaceae plants.

In particular, the methods and kits according to the invention are suitable to determine the presence of the Rlm3 blackleg resistance locus. The presence of the Rlm3 blackleg resistance locus can be determined using at least one molecular marker, wherein said one molecular marker is linked to the presence of the Rlm3 blackleg resistance locus as defined herein.

A “biological sample” can be a plant or part of a plant such as a plant tissue or a plant cell.

“Providing genomic DNA” as used herein refers to providing a sample comprising genomic DNA from the plant.

The sample can refer to a tissue sample which has been obtained from said plant, such as, for example, a leaf sample, comprising genomic DNA from said plant. The sample can further refer to genomic DNA which is obtained from a tissue sample, such as genomic DNA which has been obtained from a tissue, such as a leaf sample. Providing genomic DNA can include, but does not need to include, purification of genomic DNA from the tissue sample. Providing genomic DNA thus also includes obtaining tissue material from a plant or larger piece of tissue and preparing a crude extract or lysate therefrom.

A “kit”, as used herein, refers to a set of reagents for the purpose of performing the method of the invention, more particularly, the identification of the Rlm3 blackleg resistance genes in biological samples.

In an embodiment, the kit comprises at least one oligonucleotide for identifying the Rlm3 blackleg resistance gene of the present invention. The at least one oligonucleotide shall specifically bind to a Rlm3 blackleg resistance gene of the invention.

In a preferred embodiment of the kit of the invention comprises at least two specific primers (i.e. oligonucleotides) for identification of the Rlm3 blackleg resistance genes. The primers shall allow for amplifying the Rlm3 blackleg resistance genes as set forth herein (such as SEQ ID NO: 1, 42, 4 or 44), or fragment thereof, such as a fragment of at least 100, 200, 500 or 1000 bp. Suitable primer pairs (with two primers) are shown, for example, in Table C in the Examples section.

Optionally, the kit can further comprise any other reagent. Alternatively, according to another embodiment of this invention, the kit can comprise at least one specific probe, which specifically hybridizes with nucleic acid of biological samples to identify the presence of the Rlm3 blackleg resistance genes therein.

Optionally, the kit can further comprise any other reagent (such as but not limited to hybridizing buffer, label) for identification of the Rlm3 blackleg resistance genes in biological samples, using the specific probe.

The kit of the invention can be used, and its components can be specifically adjusted, for purposes of quality control (e.g., purity of seed lots), detection of the presence or absence of the Rlm3 blackleg resistance genes in plant material or material comprising or derived from plant material, such as but not limited to food or feed products. The zygosity status of the Rlm3 blackleg resistance genes can be determined by using alternative sets of primers and/or probes that specifically bind to the Rlm3 locus and the corresponding locus in a susceptible Brassicaceae line.

The term “primer” or “oligonucleotide” as used herein encompasses any nucleic acid that is capable of priming the synthesis of a nascent nucleic acid in a template-dependent process, such as PCR. Typically, primers are oligonucleotides from 10 to 30 nucleotides (such as 15 to 30 nucleotides), but longer sequences can be employed. Primers may be provided in double-stranded form, though the single-stranded form is preferred. Probes can be used as primers, but are designed to bind to the target DNA or RNA and need not be used in an amplification process. Typically, the probe or primer shall be capable of detecting the Rlm3 polynucleotide of the present invention (e.g. by binding to it).

The term “recognizing” as used herein when referring to specific primers, refers to the fact that the specific primers specifically hybridize to a specific nucleic acid sequence under suitable conditions, such as the conditions set forth in the method (such as the conditions of the PCR identification protocol), whereby the specificity is determined by the presence of positive and negative controls.

The term “antibody” herein is used in the broadest sense and encompasses various antibody structures, including but not limited to monoclonal antibodies, polyclonal antibodies, multispecific antibodies (e.g., bispecific antibodies), and antibody fragments as long as they exhibit the desired antigen-binding activity (i.e. antigen-binding fragments thereof). Preferably, the antibody is a polyclonal antibody. More preferably, the antibody is a monoclonal antibody. It is to be understood that the antibody as set forth herein shall specifically bind a Rlm3 polypeptide of the present invention.

The present invention further relates to an isolated polynucleotide comprising a nucleic acid sequence selected from the group consisting of:
a) a nucleic acid sequence as shown in any one of SEQ ID NOs: 1, 42, 4 and 44; and
b) a nucleic acid sequence which is at least 80, at least 85%, at least 90%, at least 95%, at least 96%, at least 97%, at least 98% or at least 99% identical to the nucleic acid sequence shown in any one of SEQ ID NOs: 1, 42, 4 and 44.

Moreover, the present invention envisages an isolated polynucleotide comprising an amino acid sequence selected from the group consisting of:
a) an amino acid sequence as shown in any one of SEQ ID NOs: 43, 5 and 45;
b) an amino acid sequence which is at least 80%, at least 85%, at least 90%, at least 95%, at least 96%, at least 97%, at least 98% or at least 99% identical to the amino acid sequence shown in any one of SEQ ID NOs: 43, 5 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 4, 42 and 44, wherein said polypeptide is capable of conferring blackleg resistance to a plant;
d) a partial sequence of any one of a) to c), wherein said polypeptide is capable of conferring blackleg resistance to a plant.

In an embodiment, the polynucleotide is operably linked to a heterologous promoter.

The present invention also relates to an oligonucleotide which specifically recognizes (i.e. binds) to the polynucleotide of the present invention. Preferably, said oligonucleotide is capable of being used as a primer or probe.

It is another object of the invention to provide a chimeric gene comprising the following operably linked genetic elements: a plant-expressible promoter, a DNA sequence coding for a Rlm3 protein, and optionally, a transcription termination and polyadenylation region functional in plant cells, wherein said DNA sequence coding for a Rlm3 protein comprises a nucleotide sequence having at least 80% sequence identity to SEQ ID NO: 42, SEQ ID NO: 4, or to SEQ ID NO: 44; or encoding a protein having an amino acid sequence having at least 80% sequence identity to SEQ ID NO: 43, SEQ ID NO: 5 or SEQ ID NO: 45.

As used herein a “chimeric gene” refers to a nucleic acid construct which is not normally found in a plant species. A chimeric nucleic acid construct can be DNA or RNA. “Chimeric DNA construct” and “chimeric gene” are used interchangeably to denote a gene in which the promoter or one or more other regulatory regions of the gene are not associated in nature with part or all of the transcribed DNA region, or a gene which is present in a locus in the plant genome in which it does not occur naturally.

“Isolated DNA” or “isolated polynucleotide” as used herein refers to DNA not occurring in its natural genomic context, irrespective of its length and sequence. Isolated DNA can, for example, refer to DNA which is physically separated from the genomic context, such as a fragment of genomic DNA. Isolated DNA can also be an artificially produced DNA, such as a chemically synthesized DNA, or such as DNA produced via amplification reactions, such as polymerase chain reaction (PCR) well-known in the art. Isolated DNA can further refer to DNA present in a context of DNA in which it does not occur naturally. For example, isolated DNA can refer to a piece of DNA present in a plasmid. Further, the isolated DNA can refer to a piece of DNA present in another chromosomal context than the context in which it occurs naturally, such as for example at another position in the genome than the natural position, in the genome of another species than the species in which it occurs naturally, or in an artificial chromosome.

Further, expression of the Rlm3 blackleg resistance gene can be modulated, such as increased by, for example, T-DNA activation tagging, or by targeted genome engineering technologies in which, for example, the endogenous promoter is modified such that it drives higher levels of expression, or in which the endogenous promoter is replaced with a stronger promoter.

Suitable to the invention is a method to produce blackleg resistant Brassicaceae plants, comprising the steps of sowing seeds from the Brassicaceae plants according to the invention comprising a Rlm3 blackleg resistance gene, growing the plants in the field, optionally spraying the plants with fungicides, and harvesting.

A further object provides the use of the polynucleotide or chimeric gene according to the invention to increase blackleg resistance in Brassicaceae, and the use of the plants according to the invention to produce oilseed rape oil or an oilseed rape seed cake, or a seed, or a crop of oilseed rape.

The sequence of the Rlm3 blackleg resistance locus can further be used to develop molecular markers linked to the Rlm3 blackleg resistance locus.

The polynucleotide according to the invention can be used to develop molecular markers for the Rlm3 blackleg resistance locus by developing primers specifically recognizing the Rlm3 blackleg resistance gene. Further, the isolated DNA can be used to identify the genomic sequence flanking the Rlm3 blackleg resistance gene, and develop primers and probes based on the genomic sequences flanking the Rlm3 blackleg resistance gene.

The present invention also provides a vector comprising the polynucleotide and/or the chimeric gene of the present invention.

Further, the present invention relates to a host cell comprising, the polynucleotide or chimeric gene of the present invention and/or the vector of the present invention. Said host cell may be any non-human host cell, such as a bacterial cell (such as E. coli or Agrobacterium tumefaciens), a yeast cell (such as a Pichia cell) or a plant cell.

Also provided by the present invention is a polypeptide encoded by the polynucleotide of the present invention. Typically, said polypeptide is an isolated polypeptide.

Moreover, the present invention provides an antibody which specifically recognizes the Rlm3 polypeptide of the present invention.

Also provided is a method of producing food, feed, or an industrial product, comprising obtaining the plant according to the invention or a part thereof; and preparing the food, feed or industrial product from the plant or part thereof. In a further object, said food or feed is oil, meal, grain, starch, flour or protein; or said industrial product is biofuel, fiber, industrial chemicals, a pharmaceutical or a nutraceutical. In a preferred embodiment, the food is an oil.

Further provided is the use of a polynucleotide comprising a nucleotide sequence as shown in SEQ ID NO: 1, SEQ ID NO: 42, SEQ ID NO: 4 or SEQ ID NO: 44, or of a polypeptide comprising the amino acid sequence of SEQ ID NO: 43, or SEQ ID NO: 5, or SEQ ID NO: 45 to identify homologous blackleg resistance genes.

Homologous blackleg resistance genes can be identified using methods known in the art. Homologous nucleotide sequence may be identified and isolated by hybridization under stringent conditions using as probes a nucleic acid comprising the nucleotide sequence of SEQ ID NO: 1, SEQ ID NO: 42, SEQ ID NO: 4, or SEQ ID NO: 44, or part thereof. Other sequences encoding Rlm3 may also be obtained by DNA amplification using oligonucleotides specific for genes encoding Rlm3 as primers, such as but not limited to oligonucleotides comprising or consisting of about 20 to about 50 consecutive nucleotides from SEQ ID NO: 1, SEQ ID NO: 42, SEQ ID NO: 4, or SEQ ID NO: 44, or its complement. Homologous blackleg resistance genes can be identified in silico using Basic Local Alignment Search Tool (BLAST) homology search with other nucleotide or amino acid sequences. Functionality of the identified homologous blackleg resistance genes can be validated using the methods described herein, such as transforming the blackleg resistance gene under control of a plant-expressible promoter in a plant not being blackleg resistant.

Also provided is a method of producing food, feed, or an industrial product, comprising obtaining the plant according to the invention or a part thereof; and preparing the food, feed or industrial product from the plant or part thereof. In a further object, said food or feed is oil, meal, grain, starch, flour or protein; or said industrial product is biofuel, fiber, industrial chemicals, a pharmaceutical or a nutraceutical.

“Crop of oilseed rape” as used herein refers to oilseed rape cultivated as a crop, such as Brassica napus, Brassica juncea, Brassica carinata, Brassica rapa (syn. B. campestris), Brassica oleracea or Brassica nigra.

The plants according to the invention may additionally contain an endogenous gene or a transgene, which confers herbicide resistance, such as the bar or pat gene, which confer resistance to glufosinate ammonium (Liberty®, Basta® or Ignite®) (EP 0 242 236 and EP 0 242 246 incorporated by reference); or any modified EPSPS gene, such as the 2mEPSPS gene from maize (EPO 508 909 and EP 0 507 698 incorporated by reference), or glyphosate acetyltransferase, or glyphosate oxidoreductase, which confer resistance to glyphosate (RoundupReady®), or bromoxynitril nitrilase to confer bromoxynitril tolerance, or any modified AHAS gene, which confers tolerance to sulfonylureas, imidazolinones, sulfonyla-minocarbonyltriazolinones, triazolopyrimidines or pyrimidyl(oxy/thio)benzoates, such as oilseed rape imidazolinone-tolerant mutants PM1 and PM2, currently marketed as Clearfield® canola. Further, the plants according to the invention may additionally contain an endogenous gene or a transgene which confers increased oil content or improved oil composition, such as a 12:0 ACP thioesteraseincrease to obtain high laureate; which confers pollination control, such as barnase under control of an anther-specific promoter to obtain male sterility, or barstar under control of an anther-specific promoter to confer restoration of male sterility, or such as the Ogura cytoplasmic male sterility and nuclear restorer of fertility.

The plants and seeds according to the invention may be further treated with a chemical compound, such as a chemical compound selected from the following lists: Herbicides: Clethodim, Clopyralid, Diclofop, Ethametsulfuron, Fluazifop, Glufosinate, Glyphosate, Metazachlor, Quinmerac, Quizalofop, Tepraloxydim, Trifluralin. Fungicides/PGRs: Azoxystrobin, N-[9-(dichloromethylene)-1,2,3,4-tetrahydro-1,4-methanonaphthalen-5-yl]-3-(difluoromethyl)-1-methyl-1H-pyrazole-4-carboxamide (Benzovindiflupyr, Benzodiflupyr), Bixafen, Boscalid, Carbendazim, Carboxin, Chlormequat-chloride, Coniothryrium minitans, Cyproconazole, Cyprodinil, Difenoconazole, Dimetho-morph, Dimoxystrobin, Epoxiconazole, Famoxadone, Fluazinam, Fludioxonil, Fluopicolide, Fluopyram, Fluoxastrobin, Fluquinconazole, Flusilazole, Fluthianil, Flutriafol, Fluxapyroxad, Iprodione, Isopyrazam, Mefenoxam, Mepiquat-chloride, Metalaxyl, Metconazole, Metominostrobin, Paclobutrazole, Penflufen, Penthiopyrad, Picoxystrobin, Prochloraz, Prothioconazole, Pyraclostrobin, Sedaxane, Tebuconazole, Tetraconazole, Thiophanate-methyl, Thiram, Triadimenol, Trifloxystrobin, Bacillus firmus, Bacillus firmus strain I-1582, Bacillus subtilis, Bacillus subtilis strain GB03, Bacillus subtilis strain QST 713, Bacillus pumulis, Bacillus. pumulis strain GB34.

Insecticides: Acetamiprid, Aldicarb, Azadirachtin, Carbofuran, Chlorantraniliprole (Rynaxypyr), Clothianidin, Cyantraniliprole (Cyazypyr), (beta-) Cyfluthrin, gamma-Cyhalothrin, lamb-da-Cyhalothrin, Cypermethrin, Deltamethrin, Dimethoate, Dinetofuran, Ethiprole, Flonicamid, Flubendiamide, Fluensulfone, Fluopyram, Flupyradifurone, tau-Fluvalinate, Imicyafos, Imidacloprid, Metaflumizone, Methiocarb, Pymetrozine, Pyrifluquinazon, Spinetoram, Spinosad, Spirotetramate, Sulfoxaflor, Thiacloprid, Thiamethoxam, 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide, 1-(3-chloropyridin-2-yl)-N-[4-cyano-2-methyl-6-(methylcarbamoyl)phenyl]-3-{[5-(trifluoromethyl)-1H-tetrazol-1-yl]methyl}-1H-pyrazole-5-carboxamide, 1-{2-fluoro-4-methyl-5-[(2,2,2-trifluorethyl) sulfinyl]phenyl}-3-(trifluoromethyl)-1H-1,2,4-triazol-5-amine, (1E)-N-[(6-chloropyridin-3-yl)methyl]-N′-cyano-N-(2,2-difluoroethyl) ethanimidamide, Bacillus firmus, Bacillus firmus strain 1-1582, Bacillus subtilis, Bacillus subtilis strain GB03, Bacillus subtilis strain QST 713, Metarhizium anisopliae F52.

The present invention also relates to a method for protecting cultivated plants in a field, wherein said plants comprise i) a Rlm3 resistance gene and/or Rlm3 protein of the present invention, and ii) at least one resistance gene conferring herbicide tolerance and wherein said method comprises applying the said herbicide to the cultivated plants in order to control weeds. In an embodiment, said herbicide is glufosinate. In another embodiment, said herbicide is glufosinate ammonium. In another embodiment, said herbicide is glyphosate. In another embodiment, said herbicide is a mixture of glufosinate, glufosinate ammonium and/or glyphosate.

Accordingly, the present invention also relates to a method for cultivating plants in a field, said plants comprising at least one gene conferring herbicide tolerance, comprising the step of applying said herbicide to said plants, wherein said plants comprise a Rlm3 blackleg resistance locus according to the invention.

Whenever reference to a “plant” or “plants” according to the invention is made, it is under-stood that also plant parts (cells, tissues or organs, seed pods, seeds, severed parts such as roots, leaves, flowers, pollen, etc.), progeny of the plants which retain the distinguishing characteristics of the parents (especially the fruit dehiscence properties), such as seed obtained by selfing or crossing, e.g. hybrid seed (obtained by crossing two inbred parental lines), hybrid plants and plant parts derived thereof are encompassed herein, unless otherwise indicated. In some embodiments, the plant parts comprise the polynucleotide or chimeric gene of the present invention.

In some embodiments, the plant cells of the invention, i.e. a plant cell comprising a Rlm3 blackleg resistance gene as well as plant cells generated according to the methods of the invention, may be non-propagating cells.

The obtained plants according to the invention can be used in a conventional breeding scheme to produce more plants with the same characteristics or to introduce the characteristic of the presence of the Rlm3 gene according to the invention in other varieties of the same or related plant species, or in hybrid plants. The obtained plants can further be used for creating propagating material. Plants according to the invention can further be used to produce gametes, seeds (including crushed seeds and seed cakes), seed oil, embryos, either zygotic or somatic, progeny or hybrids of plants obtained by methods of the invention. Seeds obtained from the plants according to the invention are also encompassed by the invention.

“Creating propagating material”, as used herein, relates to any means know in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a nucleic acid which is functionally or structurally defined, may comprise additional DNA regions etc.

All patents, patent applications, and publications or public disclosures (including publications on internet) referred to or cited herein are incorporated by reference in their entirety.

In the description and examples, reference is made to the following sequences:

### SEQUENCES

SEQ ID NO: 1: Rlm3 genome sequence
SEQ ID NO: 4: Rlm3-19a coding sequence
SEQ ID NO: 5: Rlm3-19a protein sequence
SEQ ID NO: 9: Rlm3-19a cDNA sequence
SEQ ID NO: 11: Rlm3-20 cDNA sequence
SEQ ID NO: 12: Rlm3-31 cDNA sequence
SEQ ID NO: 13 to 29: Primer sequences (see Table C in the Examples section)
SEQ ID NO: 30: Rlm4 genome sequence
SEQ ID NO: 31: Rlm4 coding sequence
SEQ ID NO: 32: Rlm4 protein sequence
SEQ ID NO: 33: Rlm7 genome sequence
SEQ ID NO: 34: Rlm7 coding sequence
SEQ ID NO: 35: Rlm7 protein sequence
SEQ ID NO: 36: Rlm9 genome sequence
SEQ ID NO: 37: Rlm9 coding sequence
SEQ ID NO: 38: Rlm9 protein sequence
SEQ ID NO: 39: AvrLm3 genome sequence
SEQ ID NO: 40: AvrLm3 coding sequence including signal peptide (AvrLm3-WT)
SEQ ID NO: 41: AvrLm3 coding sequence without signal peptide (AvrLm3-SP)
SEQ ID NO: 42: Rlm3-19 coding sequence
SEQ ID NO: 43: Rlm3-19 protein sequence
SEQ ID NO: 44: Rlm3-19b coding sequence
SEQ ID NO: 45: Rlm3-19b protein sequence
SEQ ID NO: 46: Rlm3-19 cDNA sequence
SEQ ID NO: 47: Rlm3-19b cDNA sequence

### EXAMPLES

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1—Identification of Rlm3 Blackleg Resistance Genes

The genomic region of Rlm3 was determined based on the publication of Larkan et al (2016) Single R Gene Introgression Lines for Accurate Dissection of the Brassica-Leptosphaeria Pathosystem. Front. PlantSci.7:1771. doi: 10.3389/fpls.2016.01771. Based on the publication of Larkan et al. (2016), markers linked to Rlm3 were developed based on overlaying genetic maps.

The markers were used to screen a proprietary BAC library (BNapusKEY) based on 14 superpools. 2 BAC clones containing Rlm3 were sequenced.

### Annotation of Candidate Blackleg R Genes

All genes on the BAC clone sequence were numbered and annotated based on homology with Arabidopsis. Three genes linked to disease resistance were selected for further validation.

Gene 19 shows homology to AT1G79680, WAKL10. Gene 19 has different splice variants. One longer gene (SEQ ID NO: 46) and two shorter ones (SEQ ID NO: 9 and 47). The two short ones both show homology to AT1G79680 and were named Gene 19a (or Rlm3-19a) and Gene 19b (or Rlm3-19b). The longer sequence, a duplication, is a combination of both a and b sequences, is called Gene 19 (or Rlm3-19).

Gene 20 also shows homology to AT1G79680, WAKL10. The cDNA sequence of gene 20 is shown in SEQ ID NO: 11.

Gene 31 shows homology to AT1G80840, a WRKY40 transcription factor. The cDNA sequence of gene 31 is shown in SEQ ID NO: 12.

### Example 2—Validation of Rlm3 in Loss-of-Function Approach

Mutations in genes 19a, 19b, 20, and 31 of Brassica napus identified above were generated and identified as follows:
30,000 seeds from a resistant elite spring oilseed rape breeding line (M0 seeds) were pre-imbibed for 2 h on wet filter paper in deionized or distilled water. Half of the seeds were exposed to 0.8% EMS and half to 1% EMS (Sigma: M0880) and incubated for 4 h.
The mutagenized seeds (M1 seeds) were rinsed three times and dried in a fume hood overnight. 30,000 M1 plants were grown in soil and selfed to generate M2 seeds. M2 seeds were harvested for each individual M1 plant.
Two times 4800 M2 plants, derived from different M1 plants, were grown and DNA samples were prepared from leaf samples of each individual M2 plant according to the CTAB method (Doyle and Doyle, 1987, Phytochemistry Bulletin 19:11-15).
The DNA samples were screened for the presence of point mutations in genes 19a, 19b, 20, and 31 that cause the introduction of STOP codons and another amino acid in the protein-encoding regions of genes 19a, 19b, 20, and 31, by direct sequencing by standard sequencing techniques (LGC) and analyzing the sequences for the presence of the point mutations using the NovoSNP software (VIB Antwerp).
The mutant alleles of genes 19a, 19b, 20, and 31 as depicted in Table A were thus identified.

TABLE A
STOP codon mutations in genes 19a, 19b, 20, and 31. The Nt position
is relative to the coding sequence of the genes, i.e. SEQ ID
NO: 4 for Rlm3-19a, SEQ ID NO: 44 for Rlm3-19b. The position
of the mutation in the protein is relative to the position in
SEQ ID NO: 5 for Rlm3-19a and SEQ ID NO: 45 for Rlm3-19b.

| | Mutation | Nt | WT | mut | MUT |
|---|---|---|---|---|---|
| Gene | Name | position | codon | codon | Type |
| Gene 19a | ATHR31A101 | 1735 | CGA | TGA | R-579-STOP |
| Gene 19a | ATHR31A102 | 2017 | CGA | TGA | R-673-STOP |
| Gene 19b | ATHR32A101 | 1237 | CAA | TAA | Q-413-STOP |
| Gene 19b | ATHR32A102 | 1450 | CAG | TAG | Q-484-STOP |
| Gene 20 | ATHR33A103 | 1501 | CAG | TAG | Q-501-STOP |
| Gene 31 | ATHR34A101 | 367 | CAG | TAG | Q-123-STOP |

Plants comprising these mutations were analyzed for loss of resistance to Leptosphaeria maculans Isolate Lm1033-1.

Plants were grown in a growth chamber at 14° C. with a 15-h photoperiod. Seven days old cotyledons were punctured with a needle and a droplet (10 μl) of spore suspension was added onto the puncture. Four inoculation sites per plant were applied. Symptoms were scored two times, respectively 14- and 17-days post inoculation. Scoring of the lesions was done using a rating scale from 1 to 9, 1 being susceptible, 9 being resistant. The phenotype is indicated as S when the average scoring of lesions is rated less than 5, or R when the average scoring of lesions is rated at least 5 or more. The results are shown in Table B.

TABLE B
Blackleg resistance phenotype of knock-out mutants

| | | | Disease screening | | |
|---|---|---|---|---|---|
| | | | Average | Average | |
| | | | score | score | |
| Mutant | Gene | Mutation | 14 dpi | 17 dpi | Phenotype |
| ATHR31A101 | Gene19a | R-579-STOP | 1.2 | 1 | S |
| ATHR31A102 | Gene19a | R-673-STOP | 2.7 | 1.3 | S |
| ATHR32A101 | Gene19b | Q-413-STOP | 2.4 | 1.5 | S |
| ATHR32A102 | Gene19b | Q-484-STOP | 2.6 | 1.1 | S |
| ATHR33A103 | Gene20 | Q-501-STOP | 7.8 | 7.2 | R |
| ATHR34A101 | Gene31 | Q-123-STOP | 8.0 | 7.9 | R |

(S = susceptible; R = resistant)

Each one of the four mutations of gene 19 gene (both gene 19a and gene 19b) lead to loss of resistance to Leptosphaeria maculans Isolate Lm1033-1. These results show that gene 19 is required for blackleg resistance.

### Example 3—Rlm3 Protein Annotation

The proteins encoded by gene 19, 19a and 19b were analyzed using the pfam and Interpro databases (http://pfam.xfam.org/and http://www.ebi.ac.uk/interpro/) to identify the presence of conserved protein domains that are indicative of protein function. The proteins encoded by gene 19, 19a and 19b show a high overall conservation when compared to the proteins encoded by Rlm4, Rlm7 and Rlm9. Conserved domains identified in the proteins encoded by gene 19, 19a, 19b, Rlm4, Rlm7 and Rlm9 include:
A signal peptide domain;
A wall-associated receptor kinase galacturonan-binding domain;
A wall-associated kinase domain;
A Calcium-binding EGF domain;
A transmembrane domain; and
A protein kinase domain including a Guanylyl cyclase like domain.

The presence of these conserved protein domains in the proteins encoded by gene 19, 19a and 19b further confirm that these are functional proteins, members of the WAKL (Wall-associated receptor kinase-like) gene family.

### Example 4—Validation of Rlm3 in Transgenic Approach

The coding sequences of gene 19, 19a and 19b (SEQ ID NO: 42, 4, 44) are cloned under control of a constitutive 35S promoter, and under control of their native promoters in a T-DNA expression vector. The genomic region comprising gene 19, 19a and 19b (SEQ ID NO: 1), is also cloned under control of a constitutive 35S promoter and under control of its native promoter in a T-DNA expression vector. All T-DNA expression vectors comprise a selectable marker. The resulting vectors are transformed into the Westar and/or Darmor Brassica napus cultivar, which is susceptible to blackleg, using the hypocotyl transformation protocol essentially as described by De Block et al. (1989), Plant Physiol. 91:694. The copy number of the transgene in the transgenic plant is determined by real time PCR on the bar gene. The transformed Brassica napus plants comprising the coding sequences of gene 19, 19a and 19b, or the genomic region comprising gene 19 show increased blackleg resistance as compared to untransformed Brassica napus cultivar Westar and/or Darmor, respectively.

### Example 5—Transfer of Rlm3 into Other Brassicaceae Lines

The Rlm3 genes are transferred into Brassicaceae breeding lines by the following method: A plant containing the Rlm3 genes (donor plant), is crossed with a Brassicaceae line (elite parent/recurrent parent) or variety lacking the Rlm3 genes. The following introgression scheme is used, wherein the presence of the Rlm3 genes is indicated with Rlm3, and the absence of the Rlm3 gene is indicated with—:
Initial cross: Rlm3/Rlm3 (donor plant) X−/− (elite parent)
F1 plant: Rlm3/−
BC1 cross: Rlm3/− X−/− (recurrent parent)
BC1 plants: 50% Rlm3/− and 50%−/−

The 50% Rlm3/− are selected using molecular markers (e.g. AFLP, PCR, Invader™, KASP, and the like) for the presence of the Rlm3 gene.

Further backcrosses can be performed. Upon one or more steps of backcrossing (BCx), backcrossed plants heterozygous for Rlm3 are selfed:
BCx S1 cross: Rlm3/− X Rlm3/−
BCx S1 plants: 25% Rlm3/Rlm3 and 50% Rlm3/− and 25%−/−

Plants containing Rlm3 are selected using molecular markers linked with the Rlm3 gene. Individual BCx S1 plants that are homozygous for Rlm3 (Rlm3/Rlm3) are selected using molecular markers linked with Rlm3. These plants can then be used for seed production.

### Example 6—PCR Assay Design to Assay the Sequences of Rlm3, Gene 19

Table C shows primers that were used for the studies described herein. For PCR analysis, the forward and reverse of each set were used in combination. Asterisk (*) indicates this primer should be combined with Rlm3_Gene19b_FW_set2.

TABLE C
Primers pairs designed for assaying the sequences of Rlm3

| | | | | | |
|---|---|---|---|---|---|
| | | SEQ | | | |
| | | ID | | | Amplicon |
| AssaySet | Sequence | NO | Length | Amplicon | (gDNA) |

Rlm3_Gene19a

| | | | | | |
|---|---|---|---|---|---|
| Rlm3_Gene19a_FW_set1 | AGCTCTTACAATCCTTCTTCCC | 13 | 22 | 2319 | 2556 |
| Rlm3_Gene19a_RV_set1 | AGGAAACAAAGGCTCAGTATCT | 14 | 22 | | |
| Rlm3_Gene19a_FW_set2 | CCCTTATGATTATCTTCTCTCTGC | 15 | 27 | 2311 | 2548 |
| | TAT | | | | |
| Rlm3_Gene19a_RV_set2 | CCTGTTTGGTAAGGAAACAAAGG | 16 | 23 | | |

Rlm3_Gene19b

| | | | | | |
|---|---|---|---|---|---|
| Rlm3_Gene19b_FW_set1 | AGCTCTTATAATCCTTCTTCCCTT | 17 | 25 | 2263 | 3521 |
| | T | | | | |
| Rlm3_Gene19b_RV_set1 | TACCGACGTCGTACTGAGAA | 18 | 20 | | |
| Rlm3_Gene19b_FW_set2 | CCCTTTTAATTATTTTCTCTCTGC | 19 | 32 | | |
| | TATTTTTG | | | | |
| Rlm3_Gene19b_RV_set2 | TTCTCCAATTCCATGGACAC | 20 | 20 | 2092 | 3350 |
| Rlm3_Gene19b_RV_set3* | CCAAGTAGTTATCGTGTGATCG | 21 | 22 | 1565 | 2823 |

Rlm3_OverlapGene19a&b_long

| | | | | | |
|---|---|---|---|---|---|
| Rlm3_Overlap_long_FW_set1 | GTCCAAAGTCGTGGATGAAGA | 22 | 21 | 1088 | 2307 |
| Rlm3_Overlap_long_RV_set1 | TCCCGATTCCAAATGGGTATG | 23 | 21 | | |
| Rlm3_Overlap_long_FW_set2 | TCGTGCAGTGAAAGAGAATAGG | 24 | 22 | 1010 | 2229 |
| Rlm3_Overlap_long_RV_set2 | CATTACCGTTGCAGCTTGTTT | 25 | 21 | | |

Rlm3_OverlapGene19a&b_short

| | | | | | |
|---|---|---|---|---|---|
| Rlm3_Overlap_short_FW_set1 | CTGCTCCAACTTCTCAATACGA | 26 | 22 | 198 | 1417 |
| Rlm3_Overlap_short_RV_set1 | CTCCACATTTGCTAGGACAAGA | 27 | 22 | | |
| Rlm3_Overlap_short_FW_set2 | ACAACAATACTGTTACTGCTCCA | 28 | 23 | 199 | 1418 |
| Rlm3_Overlap_short_RV_set2 | GGACAAGACCCGGCAAC | 29 | 17 | | |

### Example 7—Validation of Rlm3 in Protoplast Assays

### Protoplast Isolation and Transfection

Protoplasts are isolated from the leaves of 4- to 7-week-old aseptically grown plants. Healthy leaves are cut into fine strips with a sharp razor blade and transferred to a Petri dish. The strips are then infiltrated with cell wall-dissolving enzyme solution containing 0.25% cellulase R10 and 0.25% macerozyme R10 and incubated overnight in the dark with gentle shaking (40 rpm) at 24° C. After enzymatic digestion, the released protoplasts are collected by filtering the mixture through 40-μm nylon meshes and resuspended in W5 solution. The resuspended protoplasts are washed with W5 solution, after which the cell pellet is re-suspended in MMG solution. For transformation, 200 μl of cells (2.5×105) are mixed with 20 μg plasmid DNA and 220 μl of freshly prepared polyethylene glycol (PEG) solution. The mixture is incubated for 10 min in the dark. After removing the PEG solution, the protoplasts are resuspended in 2 ml of W5 solution, transferred into six-well plates, and incubated at 24° C. for at least 48 h.

### Expression Vectors

The coding sequences of gene 19, 19a and 19b (SEQ ID NO: 42, 4 and 44) are cloned under control of a constitutive Arabidopsis ubiquitin-10 promoter and a 3′pin2 terminator. Similarly, the coding sequence of the cognate AvrLm3 effector (SEQ ID NO: 40 and 41), with and without native signal peptide (AvrLm3-WT and AvrLm3-SP, respectively), is cloned between the promoter of the 35S gene of Cauliflower mosaic virus and a fragment of the 3′ untranslated region of 35S. The resulting expression vectors are used for protoplast transfection.

Rlm3 Locus Interacts with AvrLm3

To test the functionality of Rlm3, protoplasts of Brassica napus cultivar PPS02-144, which contains the genomic region comprising gene 19, gene 19a, and gene 19b (SEQ ID NO: 1), are transiently transfected with the AvrLm3-WT or the AvrLm3-SP effector construct along with a GFP reporter. In this assay, diminished GFP fluorescence upon recognition of AvrLm3 by Rlm3 indicates hypersensitive response-specific cell death, which is used as a measure for disease resistance.

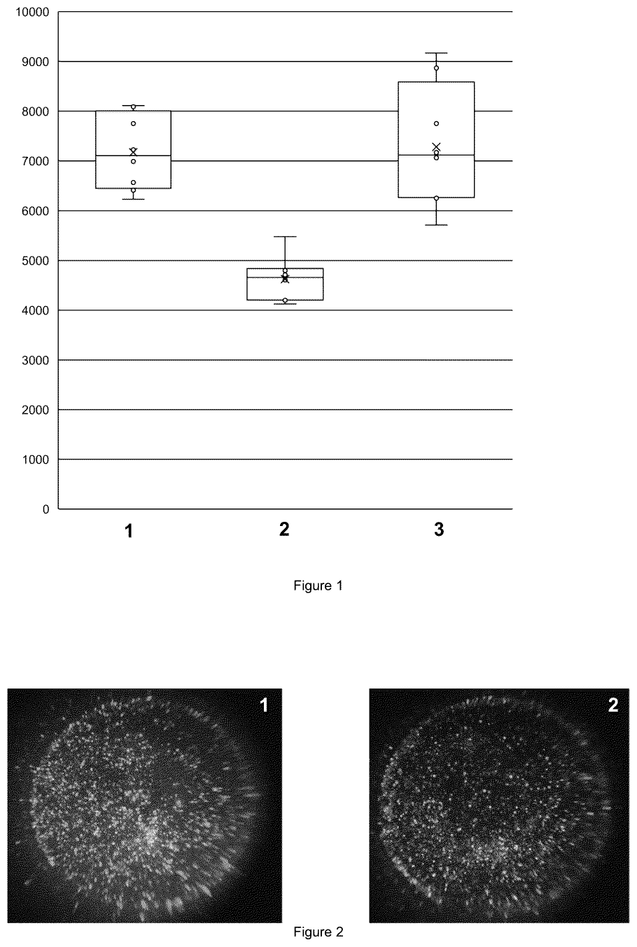

As shown in FIGS. 1 and 2, AvrLm3-WT (2) but not the AvrLm3-SP (3) effector variant lacking the native signal peptide induces a reduction in GFP signal as compared to the GFP control (1). This confirms that gene 19, gene 19a and/or gene 19b encode a cell surface receptor interacting with the AvrLm3 effector. In addition, these findings further confirm that the predicted protein domains of gene 19, 19a and 19b, as described in example 3, constitute functional protein domains.

Next, Brassica napus protoplasts of the susceptible cultivars Westar and/or Darmor are transiently transfected with either gene 19, 19a, 19b or the combination of 19a and 19b, together with AvrLm3-WT or AvrLm3-SP and a GFP reporter. A control sample, which provides a read-out of reporter activity in the absence of AvrLm3 recognition, is included as reference. Quantification of GFP reporter activity in the different samples shows gene 19, 19a, 19b and the combination thereof, confer blackleg resistance.

1. A method for producing a blackleg resistant plant comprising introducing into the genome of a plant a polynucleotide comprising a blackleg resistance locus Rlm3, wherein said Rlm3 locus comprises at least one polynucleotide having a nucleic acid sequence selected from:
a) a nucleic acid sequence as shown in any one of SEQ ID NOs: 1, 4, 42 and 44,
b) a nucleic acid sequence which is at least 80%, at least 85%, at least 90%, at least 95%, at least 96%, at least 97%, at least 98% or at least 99% identical to the nucleic acid sequence shown in any one of SEQ ID NOs: 1, 4, 42 and 44, or
c) a nucleic acid sequence having at least 80%, or at least 85%, or at least 90%, or at least 92%, or at least 95%, or at least 98%, or at least 99%, or 100% sequence identity to nt 2106-9496 of SEQ ID NO: 1, to nt 2106-4688 of SEQ ID NO: 1, or to nt 5908-9496 of SEQ ID NO: 1.

2. The method of claim 1, wherein said method comprises introducing into the genome of a plant
a) a first polynucleotide having a nucleic acid sequence which is at least 80%, at least 85%, at least 90%, at least 95%, at least 96%, at least 97%, at least 98%, at least 99% or 100% identical to the nucleic acid sequence of SEQ ID NO: 4 or to nt 2106-4688 of SEQ ID NO: 1; and
b) a second polynucleotide having a nucleic acid sequence which is at least 80%, at least 85%, at least 90%, at least 95%, at least 96%, at least 97%, at least 98%, at least 99% or 100% identical to the nucleic acid sequence of SEQ ID NO: 44 or to nt 5908-9496 of SEQ ID NO: 1.

3. A method for producing a blackleg resistant plant comprising introducing into the genome of a plant at least one polynucleotide comprising a Rlm3 associated open reading frame, wherein said Rlm3 associated open reading frame encodes a polypeptide comprising an amino acid sequence selected from:
a) an amino acid sequence as shown in any one of SEQ ID NOs: 5, 43 and 45;
b) an amino acid sequence which is at least 80%, at least 85%, at least 90%, at least 95%, at least 96%, at least 97%, at least 98% or at least 99% identical to the amino acid sequence shown in any one of SEQ ID NOs: 5, 43 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 4, 42 and 44, wherein said polypeptide is capable of conferring blackleg resistance to a plant; or
d) a partial sequence of any one of a) to c), wherein said polypeptide is capable of conferring blackleg resistance to a plant.

4. The method of claim 3, wherein said method comprises introducing into the genome of a plant
I) a first polynucleotide comprising a Rlm3 associated open reading frame encoding a polypeptide comprising an amino acid sequence selected from:
a) an amino acid sequence which is at least 80%, at least 85%, at least 90%, at least 95%, at least 96%, at least 97%, at least 98%, at least 99% or at least 100% identical to the amino acid sequence shown in SEQ ID NOs: 5;
b) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NO: 4; or
c) a partial sequence of a) or b);
and
II) a second polynucleotide comprising a Rlm3 associated open reading frame encoding a polypeptide comprising an amino acid sequence selected from:
a) an amino acid sequence which is at least 80%, at least 85%, at least 90%, at least 95%, at least 96%, at least 97%, at least 98%, at least 99% or at least 100% identical to the amino acid sequence shown in SEQ ID NOs: 45;
b) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NO: 44; or
c) a partial sequence of a) or b).

5. The method of claim 1, wherein said method further comprises
i) identifying a plant having integrated into its genome the polynucleotide comprising the blackleg resistance locus Rlm3 or the polynucleotide encoding a Rlm3 associated open reading frame; and
ii) generating progeny from said plant wherein blackleg resistance has been conferred to said progeny.

6. A method of conferring blackleg resistance to a plant comprising genetically modifying a silent allele of a gene encoding a polypeptide comprising a Rlm3 associated open reading frame such that the silent allele is capable of expressing said polypeptide, wherein said Rlm3 associated open reading frame encodes a polypeptide comprising an amino acid sequence selected from:
a) an amino acid sequence as shown in any one of SEQ ID NOs: 5, 43 and 45;
b) an amino acid sequence which is at least 80%, at least 85%, at least 90%, at least 95%, at least 96%, at least 97%, at least 98% or at least 99% identical to the amino acid sequence shown in any one of SEQ ID NOs: 5, 43 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 4, 42 and 44, wherein said polypeptide is capable of conferring blackleg resistance to a plant; or
d) a partial sequence of any one of a) to c), wherein said polypeptide is capable of conferring blackleg resistance to a plant.

7. The method of claim 6, wherein homologous recombination or a genome editing technology is used.

8. The method of claim 1, wherein said plant is a Brassicaceae plant.

9. A method for the manufacture of food, feed or an industrial product comprising:
i) conferring blackleg resistance to a plant by the method of claim 1; and
ii) preparing the food, feed or industrial product from the plant obtained in step i).

10. The method of claim 9, wherein
i) said food or feed is an oil, a meal, starch, flour, or a protein; or
ii) said industrial product is a biofuel, a fiber, an industrial chemical, a drug or a nutrient.

11. A method for assessing blackleg resistance in a plant comprising:
I) determining the presence or absence of a blackleg resistance locus Rlm3 or a polynucleotide comprising a Rlm3 associated open reading frame in a sample of said plant comprising genomic DNA, wherein
i) said Rlm3 locus comprises at least one polynucleotide having a nucleic acid sequence selected from:
a) a nucleic acid sequence as shown in any one of SEQ ID NOs: 1, 4, 42 and 44;
b) a nucleic acid sequence which is at least 80, at least 85%, at least 90%, at least 95%, at least 96%, at least 97%, at least 98% or at least 99% identical to the nucleic acid sequence shown in any one of SEQ ID NOs: 1, 4, 42 and 44; or
c) a nucleic acid sequence having at least 80%, or at least 85%, or at least 90%, or at least 92%, or at least 95%, or at least 98%, or at least 99%, or 100% sequence identity to nt 2106-9496 of SEQ ID NO: 1, to nt 2106-4688 of SEQ ID NO: 1, or to nt 5908-9496 of SEQ ID NO: 1;
and
ii) said Rlm3 associated open reading frame encodes a polypeptide comprising an amino acid sequence selected from:
a) an amino acid sequence as shown in any one of SEQ ID NOs: 5, 43 and 45;
b) an amino acid sequence which is at least 80%, at least 85%, at least 90%, at least 95%, at least 96%, at least 97%, at least 98% or at least 99% identical to the amino acid sequence shown in any one of SEQ ID NOs: 5, 43 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 4, 42 and, wherein said polypeptide is capable of conferring blackleg resistance to a plant; or
d) a partial sequence of any one of a) to c), wherein said polypeptide is capable of conferring blackleg resistance to a plant;
and
II) assessing blackleg resistance of the plant based on the presence or absence of said blackleg resistance locus Rlm3 in said plant.

12. A method for assessing blackleg resistance in a plant comprising:
i) determining the presence or absence of a polypeptide encoded by a Rlm3 associated open reading frame in a sample of said plant comprising protein, wherein said Rlm3 associated open reading frame encodes a polypeptide comprising an amino acid sequence selected from:
a) an amino acid sequence as shown in any one of SEQ ID NOs: 5, 43 and 45;
b) an amino acid sequence which is at least 80%, at least 85%, at least 90%, at least 95%, at least 96%, at least 97%, at least 98% or at least 99% identical to the amino acid sequence shown in any one of SEQ ID NOs: 5, 43 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 4, 42 and 44, wherein said polypeptide is capable of conferring blackleg resistance to a plant; or
d) a partial sequence of any one of a) to c), wherein said polypeptide is capable of conferring blackleg resistance to a plant;
and
ii) assessing blackleg resistance of the plant based on the presence or absence of said polypeptide encoded by a Rlm3 associated open reading frame.

13. (canceled)

14. (canceled)

15. A polynucleotide comprising the blackleg resistance locus Rlm3, wherein said Rlm3 locus comprises at least one polynucleotide having a nucleic acid sequence selected from:
a) a nucleic acid sequence as shown in any one of SEQ ID NOs: 1, 4, 42 and 44;
b) a nucleic acid sequence which is at least 80, at least 85%, at least 90%, at least 95%, at least 96%, at least 97%, at least 98% or at least 99% identical to the nucleic acid sequence shown in any one of SEQ ID NOs: 1, 4, 42 and 44; or
c) a nucleic acid sequence having at least 80%, or at least 85%, or at least 90%, or at least 92%, or at least 95%, or at least 98%, or at least 99%, or 100% sequence identity to nt 2106-9496 of SEQ ID NO: 1, to nt 2106-4688 of SEQ ID NO: 1, or to nt 5908-9496 of SEQ ID NO: 1.

16. A polynucleotide comprising a Rlm3 associated open reading frame, wherein said Rlm3 associated open reading frame encodes a polypeptide comprising an amino acid sequence selected from:
a) an amino acid sequence as shown in any one of SEQ ID NOs: 5, 43 and 45;
b) an amino acid sequence which is at least 80%, at least 85%, at least 90%, at least 95%, at least 96%, at least 97%, at least 98% or at least 99% identical to the amino acid sequence shown in any one of SEQ ID NOs: 5, 43 and 45, wherein said polypeptide is capable of conferring blackleg resistance to a plant;
c) an amino acid sequence encoded by a nucleic acid sequence which is at least 80%, at least 85%, at least 90%, at least 95%, at least 96%, at least 97%, at least 98% or at least 99% identical to the nucleic acid sequence as shown in any one of SEQ ID NOs: 1, 4, 42 and 44, wherein said polypeptide is capable of conferring blackleg resistance to a plant; or
d) a partial sequence of any one of a) to c), wherein said polypeptide is capable of conferring blackleg resistance to a plant.

17. The polynucleotide of claim 15, wherein said polynucleotide is operably linked to a heterologous promoter.

18. An oligonucleotide which specifically hybridizes to the polynucleotide of claim 15 and which is capable of being used as a primer or probe.

19. A vector or gene construct comprising the polynucleotide of claim 15.

20. A host cell comprising the polynucleotide of claim 15.

21. A plant comprising the polynucleotide of claim 15.

22. The plant according to claim 21, further comprising in its genome at least one additional blackleg resistance gene selected from Rlm4, Rlm7 and/or Rlm9.

23. A polypeptide encoded by the polynucleotide of claim 15.

24. An antibody which specifically recognizes the polypeptide of claim 23.

25. A kit for assessing blackleg resistance in a plant comprising the oligonucleotide of claim 18.

26. A method to determine the presence or absence of a polynucleotide of claim 15 in a biological sample, comprising providing DNA from said biological sample, and analyzing said DNA for the presence or absence of said polynucleotide.

27. A method to determine the presence or absence of a polypeptide of claim 23 in a biological sample, comprising providing polypeptides from said biological sample, and analyzing said polypeptides for the presence or absence of said polypeptide.

28. A method for protecting cultivated plants in a field, wherein said plants are plants of claim 21 further comprising at least one resistance gene conferring herbicide tolerance and wherein said method comprises applying the said herbicide to the cultivated plants in order to control weeds.

29. The method of claim 28, wherein said herbicide is glufosinate, glufosinate ammonium, glyphosate or a mixture thereof.


SEQUENCE LISTING (47 sequences)

SEQ ID NO: 1 - DNA, 9957 bp, Brassica napus
  source 1..9957: mol_type = unassigned DNA
aaaaagattggcaacaataataacaaacatgcaacggtcattttttttgttttgtttgtggtgagtttcattagctgcatgtaggttttcatacaactcgcatcaatcttcgactcagttgtccatttcggttatgaaaataccaacttaaatatataaaatcatacgagctgataatttgattagacctttcacaacttatttagtgtgatctatatatagcgcttatttagtcaattacggatctgttgatttggttctgtcttctatttagacattgatgaatgttagggaggaaaagggctaaaaactgtggagaacaaacttatgtgaacgttccatcaaggattttttattcatattcttaagtcatataacataacaaaaaggagcgatctttaactattgctgtcatatattgattactatttacgttatgaaatagagaagatcaagcatgtggttcaaggtgaatctctatctaggtgaatctctccgtataaagtaggatatggttcaaaaactgagactgctatagactataggtaaatttggtataggatcgtcatcacaagctatgtgtacaacataagcgagtcaaggctttggatcatctatccacactagttgctacatgtttatcagcagcgtttccatacataatgagaatcgtattgtggcagtagctatgttgaaagtatctcggatttgcttagttgacttccactaaccggacactcctcacgaccaagatttacacattcacattaacaagggacacattttagaaaaattatactctctccgtttctaaataagtgccactttgatattttttacacatattaagaaagtggtgaaaatatatataagttgttatgaattaaacatctttcaccaataatatttgagataaataaaattatttataaaatcaatgcagtttttaattaattttcagttgaaaataagtataatttgcattgaaattataaagtgacaatttttgtaacaaaaaaataaaatgacatttattatgaaaataggaagtatatactccctccgtatcactttaaatggtgtttagaatttttttttatttcaaaataattgatgttttcattattgttgtagagtttaatatcatttgaactttgtgactaattacaaaatactgaattttttttattggttgaattagtttcatttaatgatatttttatataattaaaataaattgtatagaagtttgtattttcttaattcatgtgcaaaaaccttaaaattcacttaaagtgatacagagagaatatatacaagcgatatatgcatttctttcgtcaaaaaggtgcatttacactcaaaacttctatttttataattaatcgaaggcaaatatgtctcttaaatataatagatatattttaatattttaatattttcataattttaattttaaattttttattaaatatttttacttttgtatatatattgttttaagaaaaaagaaaaaaaaatttaccttcccgcaaccgcaaacgctagttggagccagcttttgaatttatgagattcggagcggtttgaagcaatttagagcgatttaagtgattgttgactgttgtaaaccgccgacaaccgctaccaaccgtaaaagctgcgtttgcgggtggtagcgggaaaaccagtcgccccctaaatatactccgttaagattctaatcatgaccgaccataaatgatggttagggtctctgatttggactatgcgaaaagaattggatatccgtctctttcgcttggtgcaaaatttcttgcaatggactccaccctttgtctatgactatatgttgatgaaaaaaaatcaaaagatggagcccgacttgacatgctttaagtaggctgtaggcctaccgatataacagctgcgttgatttagttgaaggaaacataaaagagagaaagaaaaaataggagaataaggtaaaacgaagacttaagactggctgtgtccaagtaaacggatgcatttatcttctgtctcttctctgcaaaagccagtcttttagtaatacgggggaaataaaaaaatttcttgagaagatgttaaacatgagctcttacaatccttcttcccttatgattatcttctctctgctatttttcttggtctttgatctcacttttgccgggtcttgtcctaacacttgtggaggaatcgacatcccgtacccatttggaatcggggagggatgctatctgaataaatggtacgaaattaaatgtgatcataataactctctttcagtttcagggaagcgtgtccctattctttccttaatagccaaggaagttgtggatatctctcttccggaatcacttggctcgtatggttattattctcgcatgccatacgggaaaattcacataaaacacccaatagcttcaacggggtgctccagcaacaaacaagagtctagatcacttttgaatttgacagggagtcctttttacgtcggtgtcagtaataaactgataggtattggttgcaacagcacagcctcgttgacgaatgtcgagccaagcatcattggatgcttctccagttgcctggttcttactgctacaaaaaattatctagcgccagtcgattgtaatactacgaatgactacgggtattgtggtgaaagaagctgcaacggtaatggatgctgcaaggcaagcatgtccggtagtattcaacaggttgttggtgtcagaatagatgacaacgccaccacaacaggatggtgtaaagttgctttcttgtcaaatgaggaatacactttgtcagacagttctgatccagatcgggtatttgctaaacgatattcaacagttgagctaggatggttcatccatacaacgaatctttccttcttaggctctctaaaatgtgcaactatgaaagaatacctaaatctaagtcgaaactcatatgaatcgacttacggaataggttgtgcatgcgaccataactcctattttcctaactatgcaagttgcagctgcaatagtggctacatgggcaacccatacattctgaatggatgtaaaggttagtaagtgtttacagttcatctttatgttctagtgttatatataagactatgattaagtaaactatgagatcatcatggttgatttcagtttcattttttttttctttctaaaccagataaaaatgaatgcactgaaaaagacttgtactgtggggaggacgccacttgtgtcaatttgcaaggatcctataaatgtgtgtatagaaattaccggcttgctataggtaaaccgttcaactttctatctcaaatacacctaattttaagatattgtatcgacatgtatttaatcattcagccaaagccatgcaagaaaataaatttggttttaatttttgcagggatttgtgcaagtttcggctcattgatctttattggtggaatatgttggttatacaagtttattacaaaacaaaggaggttaaaccaaaagaagaagttcttcaagcgtaacggaggcttattgttgcaacaacaattaacttcaacccaaggcagcaatgaaaagacaagagttttcagctcaagagagctggaaaatgccactgagaactttggtttgactagaatacttggccagggaggtcaaggtaccgtctacaagggaatgcttgttgatggaagaattgtagcagtgaaaaagtccaaagtcgtggatgaagacaagctggaagagttcatcaatgaggttgtcattctctcccagatcaaccacagaaacatcgtgaagcttttagggtgttgccttgagacagatgttcctattctcgtctatgagtttattcccaatggaaaccttttcgagcatcttcatgaagatgagtcagataataatacattgaccacttgggaagtgcgtctccgcattgctatagatattgcaggagctctttcatatttgcattcagctgcttcatctccaatatatcacagggacatcaagtctacaaacataatgttggacgagaaatatcgagctaaagtgtctgattttggcacttcaagaacggtaacggtggatcatacacaccttacaacagtagtgtcaggtacagtgggatatgtggatcctgagtatttccagtctagtcagttcacagataaaagcgatgtttacagctttggagtggtgcttgttgagcttattaccggagaaaaatcagtttccttcttgcggtctcaagaaaataggacactggcaacttacttccttcgtgcagtgaaagagaataggctgttcgacattatcgatgcacgaatcagagatggctgcaagctgagtcaagtgaatgcaaccgcaaaccttgcaaggaagtgtttgaatctgaaagggaggaaacggccaagtatgagagaaacttccatggaattggagaaaattcgttcgtcttctgtggatatgaaactacatgagcatggtagtgaaaacaaggaagacaataaaaaaggagttgtggatgctaacataggggtacaatcatggaacaacaatactgttactgctccaacttctcaatacgacgtaggtacgtgttttttgtcagtgtcagatactgagcctttgtttccttaccaaacagggtaagaatttcaacctttcttaccaccgcaattatctaatcccttttcttttccgtccgatttctgtgtgtgtgtgaatgtattttgtttttaagaatacctttgaatgtgtaataaacggatctcgtctatatcaactctaaaatatacagtttctctgaatgtatatgtgtaatataaaagaataatttcataatctattacgaagactggcaatatttttctgatgatgaattttaatagctgcacatgggttctcatacgggtatatattattagaaacaaaccattccacattttaggttaaacaaaacattaagtagcctgtaagatagatagccaaccaacttattacccattgattttaggtttgaaagccatctaggttaatatggtatcagattccaatccattcagctcaacccgatccacatctatcaatggaaaagttgtctcatcaattagttcccaaacattccgaaggagtattagagataaagtttcccgcatccgaaactggaaaaaacattaactattatataaaaatagatgagtcacacaacttattagcaatcggttttagattgaaaattcatctaccttaacatatatcatgtaataatatgtctaaatcacaaaattagtgataaaatcttataccaataataactctcgatactttttttcttaataagcaaagacaaataagctagaagcacatctgtgaatgtgagtgctaaaacaggaaaaccatttcgttaaattgaacgatctaataagcgaaaggattagtctgtaccgtagtgcttgtggtcactatttaaaacttagtcatcacgccaaagttagtggactcgacctattcttgtctgtatttatgtctgtaaaatatcttgcaatggataggagaagctgtcgttttcagttttgaaatggaatccacccccttttttatgtttacatttagttgatgaagaaaattcaaaagatggagccgacgtgacataggcctaccgaaataaaactgcgttgatttagttgaaagaaaaagagagtatggacttagccgtaagcgaagacttaacacgcaaagacttttaacacctagtgttggctgtgtccaagtaaacggatgcatttatcttctatttgttccttacaaaacccagtcaccttttactaaagaaaatcatacaccggaaataacaaattttcttgagaagatgttaaacatgagctcttataatccttcttcccttttaattattttctctctgctatttttgttgatcttagatctcaccgttgccgggtcttgtcctagcaaatgtggaggaatcgacatcccatacccatttggaatcgggaagggctgctatctcaataaatggtatgaaattaaatgtgatcacaataactctttttcagtttcagggaagcctgtccctgttctttcaatacccgacaaggaagttgttaatatctctcttccgagtcatgtccactcagattatcctattcgcatgacatacggggcagttcgcataaaataccaaataacttcaaggggatgctccagcaacgaacaagagcctacatcacttttgaatttgatggggagccctttttacgttgctgacagtaatacaatgatagcgattggttgcaacaacagggcatcgctgacaaatatggagccaagtatcgtgggatgcttctccagttgcctctcaacaaaccatattgcttcgaatgattatctagacacagtcaattgttatagtaagagtgactccgatgacgcctacgagtactgtagtgaaagaagatatataaatgaaacaagctgcaacggtaatggatgctgcaaggcaagcatgcccagtagtgatcaacaagttgttggtgtcagaatagatgataactccgccgcaacaagagggtgtaaagttgccttcttaacagatgaggcctactctttattagacggaactgatccaaaactgcttcttgctaaacgatattctacagttgagctacagtggttcattgacacaacgaatctttcgtttgtagactctctaagatgcataactatgaaagagtacctacctataagtcgacaatactcacatggaatgaataactatgcaagttgtgcatgcgactttaattcttctgttcctaactacgcaagctgcagctgcaatattggttacaggggcaacccatacattctgggtggatgcaaaggttagtaagcgtgtttatagttaattatcttttttcaagtcttttatcaacttaaactattagatcaacatggctgattccagtttctctctctttttaaaccagatattagtagtgaatgccctaattgcgtgtatagatatcacgtgcagcttgtaataggtaagccgtctttccatctcaaatacacttgccccatttaagcctcatcgttttttttatattagatgcatgctacaagtgaatgcaaatgatcatgttgtaagtttgaaaactattgcattaacatctagatgtatttatttggatgcgaaattcatactatctagatatatttgagatacaaaaatatgaagaatctggatgatacatttgcatctcaaaatccaaaaaacttaaatgtccgttaatacaaattaaaacaactaatttatattaatagttattaagagatatatattttaaaaacataaaatcatgttctcagtcaaaacagtaaaattagattttaattcaaaaaccgtaaaattgatttttcgcaaaaatcctcaaaatcaaaaaatttcactgaaatagtaaaattgagcttttttataccaaagcgtgaattgagtttttctacaaaaagcgtaaattaagtttttcccttaaaacaacaaaattatatatatatatatatatatatatatatatatatatatatatatattgcaagatcatatttttttcacaaactctcaaaatcacgttttgatgaaaccgtaaaataatatttttcgtcaaaaccgcaaaaccgcatttttttcaccaaaagtataaaatcatgtttctactttaaacctgaatagtttttttgatattgaatttttatgggttttaaaattaagatctattcggatattataaactttttaatggggtttgaatctttccagatccgaatagattcatatgaacctaaaaatatctaaataatttatatatttaaaaatatcattaaataatttataaaaaaaaattaaaatccgcgcaggcgcgcggatcaagctttatgaaactaaaacaacaacttagcatctagatgtatgtagatgtaaatactaactacaaatacaaacacatttggatataacatccagatgtgtattcagatgttgtatctcgatactgcaaccaaacacagaaatgaatatggctttgaataaaggtgatattgaatatggctaatgatattgaataaaggtgatttttgacatttatttaatcatttacctaacgtaatttggttttcatttttcctctgtggggtttgcagggattggttcaagtttcggctcattgattttcatttgtggaatatacggattatacagacttattagaaaacaaaggagtttaaaacaaaagaagaagttcttcaagcgtaatggaggcttgttgttgcaacaacaattaacttcaacccaaggcaacgttgaaaagacaagagtgttcagctcgagagagctggaaaaggccaccgaaaacttcagtttaactagaatacttggccaaggcggtcaaggaaccgtctacaaaggaatgcttgttgatggaagaattgtagcagtgaaaaagtccaaggtggtggatgaagacaagttggaagagttcatcaacgaggttgtcattctgtcgcagatcaaccacagaaacattgtcaaacttttagggtgttgcctcgagacagatgtcccggttctcgtctacgagtttattcccaatggtaacctttttgaacatcttcatgaagatgagtcagacgatcacacgataactacttgggaagtgcgtctccgcattgctattgatatcgcaggagctctttcatatttgcattcagccgcatcatctccaatatatcatagggacgtcaagtctacaaacataatgttggatgagaagtatcgagctaaagtgtctgattttggcacttcaagaacagtaacggtggatcatacacaccttacaacggtagtttcaggtacggtggggtatgtggatcctgagtatttccagtctagccagttcacggacaaaagtgatgtttacagctttggtgtggtgcttgcggagcttatcaccggagaaaaatcagtttctttcttgcggtctcaagaaaataggacattggcaacttatttctttcttgcgatgaaagagaataggctgcttgacattatcgatgcacggatcagagatggttgcaagctgaatcaagtgactgcaacggcaaatctcgcaaggaagtgtttgaatctgaaaggaaggaagcggccaagtatgcgagaagtgtccatggaattggagaaaattcgtgggtcttcggtggatatggaactacatgagtgtggtagtgaaaacgaggaagagaatgaagaacaaattgtggatgctaacatagagttacaatcatggaacaacactgccgttagtgctccaacttctcagtacgacgtcggtacatcatctttgtcaatgtcagatgttgagcctctatttccttttcaaacacggtaataatttcaactttgcataccacgacattatctagtgcctgccttttgctttcccgtcctagtttggtgtttctgtatgtgtgatgtatttcttttgtgtaataaatacctatgattgtgtaataaaaggatctcgtttgtaacacagaagtttacattctgataagccagtctttaggctgttgttagccattgcattagagggacaaacataaaataatgcttatcagctttggctgaaagtctacaagaatttcagtttccccttagaaaaactggcttgaacatgttccgtttgacacacatcgccttctctttccccatcatccctgaaaacgacaagtgcctcgaacctaatgtgacagcaaagtaaatataaatggtgaaacggaatgccaaaaggaccacagctgttaaaattaggaatcgatgcctctgggcaaacagtagagcagctaccca

SEQ ID NO: 2-3 - skipped (2 entries)

SEQ ID NO: 4 - DNA, 2346 bp, Brassica napus
  source 1..2346: mol_type = unassigned DNA
atgttaaacatgagctcttacaatccttcttcccttatgattatcttctctctgctatttttcttggtctttgatctcacttttgccgggtcttgtcctaacacttgtggaggaatcgacatcccgtacccatttggaatcggggagggatgctatctgaataaatggtacgaaattaaatgtgatcataataactctctttcagtttcagggaagcgtgtccctattctttccttaatagccaaggaagttgtggatatctctcttccggaatcacttggctcgtatggttattattctcgcatgccatacgggaaaattcacataaaacacccaatagcttcaacggggtgctccagcaacaaacaagagtctagatcacttttgaatttgacagggagtcctttttacgtcggtgtcagtaataaactgataggtattggttgcaacagcacagcctcgttgacgaatgtcgagccaagcatcattggatgcttctccagttgcctggttcttactgctacaaaaaattatctagcgccagtcgattgtaatactacgaatgactacgggtattgtggtgaaagaagctgcaacggtaatggatgctgcaaggcaagcatgtccggtagtattcaacaggttgttggtgtcagaatagatgacaacgccaccacaacaggatggtgtaaagttgctttcttgtcaaatgaggaatacactttgtcagacagttctgatccagatcgggtatttgctaaacgatattcaacagttgagctaggatggttcatccatacaacgaatctttccttcttaggctctctaaaatgtgcaactatgaaagaatacctaaatctaagtcgaaactcatatgaatcgacttacggaataggttgtgcatgcgaccataactcctattttcctaactatgcaagttgcagctgcaatagtggctacatgggcaacccatacattctgaatggatgtaaagataaaaatgaatgcactgaaaaagacttgtactgtggggaggacgccacttgtgtcaatttgcaaggatcctataaatgtgtgtatagaaattaccggcttgctatagggatttgtgcaagtttcggctcattgatctttattggtggaatatgttggttatacaagtttattacaaaacaaaggaggttaaaccaaaagaagaagttcttcaagcgtaacggaggcttattgttgcaacaacaattaacttcaacccaaggcagcaatgaaaagacaagagttttcagctcaagagagctggaaaatgccactgagaactttggtttgactagaatacttggccagggaggtcaaggtaccgtctacaagggaatgcttgttgatggaagaattgtagcagtgaaaaagtccaaagtcgtggatgaagacaagctggaagagttcatcaatgaggttgtcattctctcccagatcaaccacagaaacatcgtgaagcttttagggtgttgccttgagacagatgttcctattctcgtctatgagtttattcccaatggaaaccttttcgagcatcttcatgaagatgagtcagataataatacattgaccacttgggaagtgcgtctccgcattgctatagatattgcaggagctctttcatatttgcattcagctgcttcatctccaatatatcacagggacatcaagtctacaaacataatgttggacgagaaatatcgagctaaagtgtctgattttggcacttcaagaacggtaacggtggatcatacacaccttacaacagtagtgtcaggtacagtgggatatgtggatcctgagtatttccagtctagtcagttcacagataaaagcgatgtttacagctttggagtggtgcttgttgagcttattaccggagaaaaatcagtttccttcttgcggtctcaagaaaataggacactggcaacttacttccttcgtgcagtgaaagagaataggctgttcgacattatcgatgcacgaatcagagatggctgcaagctgagtcaagtgaatgcaaccgcaaaccttgcaaggaagtgtttgaatctgaaagggaggaaacggccaagtatgagagaaacttccatggaattggagaaaattcgttcgtcttctgtggatatgaaactacatgagcatggtagtgaaaacaaggaagacaataaaaaaggagttgtggatgctaacataggggtacaatcatggaacaacaatactgttactgctccaacttctcaatacgacgtaggtacgtgttttttgtcagtgtcagatactgagcctttgtttccttaccaaacagggtaa

SEQ ID NO: 5 - protein, 781 aa, Brassica napus
MLNMSSYNPSSLMIIFSLLFFLVFDLTFAGSCPNTCGGIDIPYPFGIGEGCYLNKWYEIKCDHNNSLSVSGKRVPILSLIAKEVVDISLPESLGSYGYYSRMPYGKIHIKHPIASTGCSSNKQESRSLLNLTGSPFYVGVSNKLIGIGCNSTASLTNVEPSIIGCFSSCLVLTATKNYLAPVDCNTTNDYGYCGERSCNGNGCCKASMSGSIQQVVGVRIDDNATTTGWCKVAFLSNEEYTLSDSSDPDRVFAKRYSTVELGWFIHTTNLSFLGSLKCATMKEYLNLSRNSYESTYGIGCACDHNSYFPNYASCSCNSGYMGNPYILNGCKDKNECTEKDLYCGEDATCVNLQGSYKCVYRNYRLAIGICASFGSLIFIGGICWLYKFITKQRRLNQKKKFFKRNGGLLLQQQLTSTQGSNEKTRVFSSRELENATENFGLTRILGQGGQGTVYKGMLVDGRIVAVKKSKVVDEDKLEEFINEVVILSQINHRNIVKLLGCCLETDVPILVYEFIPNGNLFEHLHEDESDNNTLTTWEVRLRIAIDIAGALSYLHSAASSPIYHRDIKSTNIMLDEKYRAKVSDFGTSRTVTVDHTHLTTVVSGTVGYVDPEYFQSSQFTDKSDVYSFGVVLVELITGEKSVSFLRSQENRTLATYFLRAVKENRLFDIIDARIRDGCKLSQVNATANLARKCLNLKGRKRPSMRETSMELEKIRSSSVDMKLHEHGSENKEDNKKGVVDANIGVQSWNNNTVTAPTSQYDVGTCFLSVSDTEPLFPYQTG

SEQ ID NO: 6-8 - skipped (3 entries)

SEQ ID NO: 9 - DNA, 2499 bp, Brassica napus
  source 1..2499: mol_type = unassigned DNA
tctcttctctgcaaaagccagtcttttagtaatacgggggaaataaaaaaatttcttgagaagatgttaaacatgagctcttacaatccttcttcccttatgattatcttctctctgctatttttcttggtctttgatctcacttttgccgggtcttgtcctaacacttgtggaggaatcgacatcccgtacccatttggaatcggggagggatgctatctgaataaatggtacgaaattaaatgtgatcataataactctctttcagtttcagggaagcgtgtccctattctttccttaatagccaaggaagttgtggatatctctcttccggaatcacttggctcgtatggttattattctcgcatgccatacgggaaaattcacataaaacacccaatagcttcaacggggtgctccagcaacaaacaagagtctagatcacttttgaatttgacagggagtcctttttacgtcggtgtcagtaataaactgataggtattggttgcaacagcacagcctcgttgacgaatgtcgagccaagcatcattggatgcttctccagttgcctggttcttactgctacaaaaaattatctagcgccagtcgattgtaatactacgaatgactacgggtattgtggtgaaagaagctgcaacggtaatggatgctgcaaggcaagcatgtccggtagtattcaacaggttgttggtgtcagaatagatgacaacgccaccacaacaggatggtgtaaagttgctttcttgtcaaatgaggaatacactttgtcagacagttctgatccagatcgggtatttgctaaacgatattcaacagttgagctaggatggttcatccatacaacgaatctttccttcttaggctctctaaaatgtgcaactatgaaagaatacctaaatctaagtcgaaactcatatgaatcgacttacggaataggttgtgcatgcgaccataactcctattttcctaactatgcaagttgcagctgcaatagtggctacatgggcaacccatacattctgaatggatgtaaagataaaaatgaatgcactgaaaaagacttgtactgtggggaggacgccacttgtgtcaatttgcaaggatcctataaatgtgtgtatagaaattaccggcttgctatagggatttgtgcaagtttcggctcattgatctttattggtggaatatgttggttatacaagtttattacaaaacaaaggaggttaaaccaaaagaagaagttcttcaagcgtaacggaggcttattgttgcaacaacaattaacttcaacccaaggcagcaatgaaaagacaagagttttcagctcaagagagctggaaaatgccactgagaactttggtttgactagaatacttggccagggaggtcaaggtaccgtctacaagggaatgcttgttgatggaagaattgtagcagtgaaaaagtccaaagtcgtggatgaagacaagctggaagagttcatcaatgaggttgtcattctctcccagatcaaccacagaaacatcgtgaagcttttagggtgttgccttgagacagatgttcctattctcgtctatgagtttattcccaatggaaaccttttcgagcatcttcatgaagatgagtcagataataatacattgaccacttgggaagtgcgtctccgcattgctatagatattgcaggagctctttcatatttgcattcagctgcttcatctccaatatatcacagggacatcaagtctacaaacataatgttggacgagaaatatcgagctaaagtgtctgattttggcacttcaagaacggtaacggtggatcatacacaccttacaacagtagtgtcaggtacagtgggatatgtggatcctgagtatttccagtctagtcagttcacagataaaagcgatgtttacagctttggagtggtgcttgttgagcttattaccggagaaaaatcagtttccttcttgcggtctcaagaaaataggacactggcaacttacttccttcgtgcagtgaaagagaataggctgttcgacattatcgatgcacgaatcagagatggctgcaagctgagtcaagtgaatgcaaccgcaaaccttgcaaggaagtgtttgaatctgaaagggaggaaacggccaagtatgagagaaacttccatggaattggagaaaattcgttcgtcttctgtggatatgaaactacatgagcatggtagtgaaaacaaggaagacaataaaaaaggagttgtggatgctaacataggggtacaatcatggaacaacaatactgttactgctccaacttctcaatacgacgtaggtacgtgttttttgtcagtgtcagatactgagcctttgtttccttaccaaacagggtaagaatttcaacctttcttaccaccgcaattatctaatcccttttcttttccgtccgatttctgtgtgtgtgtgaatgtattttgtttttaa

SEQ ID NO: 10 - skipped

SEQ ID NO: 11 - DNA, 2590 bp, Brassica napus
  source 1..2590: mol_type = unassigned DNA
ataacaaacatcttaaaaaatgagctcttataatctttcttcccttttcattatcttttctctaagatttttcttgatctttgatctcactgttgccgtgtcttgtcctagccgttgtggtggaatcgacatcccgtacccatttggaatcggggagggatgctatctcaacaaatggtacgaaattaaatgtgatcataatacctctctttcagtttcagggaatttcgtccctgttctttccataatcggcaaggaagttgtaaatatctctcttccgagtccactccgcacgtatctttctgatcgagtaccgtacgggacagttcgcattaaaaaccaaatgacatcaaagggatgctccagcgacgaaaaagagtctacatcacttttgaatttgacggggagccctttttaccttggtgtcaataatacactgacagctattggttgcaacaacagggcgtccttgacacatgtcgagccaagcaccgtgggatgcttctccagttgcctcccaacaaaccatactgcttcaaatgattacctagagacagtcaattgtaatactaagagtgcgtacgatagcgatggctatgggtattgtagtcaaagaagctatataaatgaaacaagctgcaacggtattcgatgctgcaaggcaagcatgccccgtagtattcaacaggtagttggtgtcacaatagaagataccgccaccacaagaggagagtgtaaagttgccttcttaacagatgatgaggcatattcttttttagacggatctgatccatatctccttgctaaacgatattcaacagttgagttaggatggtttgtccatacaacgaatcttttttccttcgtagagtctctaagatgtatgcctatgaaagagtacctaaatctaagtctaagtcgacacttatacgaattgagtaagggaataagttgtgcatgcgactataattcttattttcctaacgatacaagttgcagatgcaatagaggttacaggggaaacccatacatcccaggtggatgtaaagatattaacgaatgcactgaatatgggcccttgatctgtgaggacgctacttgtgtcaatgttcaaggagactataaatgcgtgtatagaaatcatcggcttgctatagggtttggctctagtttcggctcattgatctttattgctggaatatacgggttatacagatttattagaaaacaaaggaagttaaaccagaaaaagaagttcttcaagcgtaatggaggtttattgttgcaacaacaattaacttcaacccaaggcaacgttgaaaagacaagagtgttcagctcgagagagctggaaaaggccaccgaaaacttcagtttaactagaatacttggccaaggcggtcaaggaaccgtgtacaagggaatgcttgttgatggaagaattgtagcagtgaaaaagtccaaagttgtggatgaagacaagttggaagagttcatcaatgaggttgtcattctctcccagatcaaccacagaaacatcgtcaaacttttagggtgttgccttgagacagacgtccctgttctagtctacgagtttattcccaatggcaacctttttgaacatcttcatgaagatgagtcagacgatcacacgataactacttgggaagtgcgtctccgcattgctatagatattgcaagtgctctttcatatttgcattcagctgcatcatctccgatatatcacagagacgttaaatctacaaacataatgttggatgagaaatatcaagctaaagtgtctgattttggcacttcaagaacagtaacggtggatcatacacaccttacaacggtagtttcaggtacggtgggatatgtggatcccgagtatttccagtctagccagttcacggataaaagcgatgtttacagctttggggtggtgcttgtggagcttatcactggagaaaaatcagtctctttcttgcgctctcaagaaaataggacattggcaacttatttcattattgcgatgaaagagaataggctgcttgacattatcgatgcacggatcagagatggttgcaagctgaatcaagtgactgcgacggcaaaccttgcaaggaagtgtttgaatcttaaagggaggaaacggccaagtatgcgagaagtgtccatggaattggagaaaattcgtgggtcttcggtggatatggaactacatgagtgtggtagtgaaaacgaggaagagaatgaagaacaaattgtggatgctaacatagagttacaatcatggaacaacactgccgttactgctccaacttctcaatacgacgtcggtacatcatctttgtcaatgtcagatgttgagcctctgtttccttttcaaacacggtaataatttcaactttgcatcccaccgcattatctaaggtctattgcttttccgtccaattttggtgttgtgtgtgttaaggcattttatttttctgaatacctctgaatgtgtaataaatttatctcgattgtaccaactctctaagctagacacttcctaatacataagaataatttcatagtatattac

SEQ ID NO: 12 - DNA, 1222 bp, Brassica napus
  source 1..1222: mol_type = unassigned DNA
tagtttattacatgaaaaagcatatgctgctcacgtctctctttccacctagtttcctctttaacacttcactttatatatagaatcaaaaatcattccttccttccacaattccagaagagtaaaaaacaagaacccaataagatcgactctttccaaaaatctatcaactttttcctcagcttaaggaactcccacacatatacacatacccatcctcaagaatcaagatccaagcagagtcatccatggaccagtcttcatcatcttctttcgtcgatacttctctagatctcactattggtgttactcgtatgcgagttaaagaagattcaacgacaagtgctttggtggatgaattaaaccgagtgagtgctgagaacaagaagctctcggagatgctaactttaatgtgtgaaaactacaacgtcttgaggaaacagctgatggaatatgttaacaagagcaacaacatgacagagagagaccaaaccagtcctcccaagaaacgcaaatctccagcgagagatgacgcagttagctctgcggttattggtggagtgtcagagagtagctctacggatcaagatggtcagtatctgtgcaagaagaagcagagagaagagactgttgtgaaggaaaatgtctcaagggtttattacaagaccgaagcttctgacactaccctcgtagtgaaagatgggtatcaatggaggaaatatggacagaaagtgactagagacaacccatctccaagagcttacttcaaatgtgcttgtgctccaagctgttctgtcaaaaagaaggttcagagaagtgtggaggatcagtccgtgttggtagctacttatgaaggtgaacataaccatccaatgccgtcaaagattgattcaaacaacggcttaaaccgctacatctctcttggtggtcacactgcaccagccaaaggaagtagtagtttggctgagcctgtgactgaatccaagaaagtaactagcccatcaagaatggatattccagaagttcagaaacttttggtggagcaaatggcttgttccttgacaaaagatcctaacttcacagcagcattagcagcagctgtcaccggaagattgtatcaacagaaccagaccgaaaaatagttttttagtttcaaattctattagtatttttttagatttgaatttggcatatgagacaaagagagtagaatatattatat

SEQ ID NO: 13 - DNA, 22 bp, synthetic construct
  source 1..22: mol_type = other DNA
  misc_feature 1..22: note = Primer
agctcttacaatccttcttccc

SEQ ID NO: 14 - DNA, 22 bp, synthetic construct
  source 1..22: mol_type = other DNA
  misc_feature 1..22: note = Primer
aggaaacaaaggctcagtatct

SEQ ID NO: 15 - DNA, 27 bp, synthetic construct
  source 1..27: mol_type = other DNA
  misc_feature 1..27: note = Primer
cccttatgattatcttctctctgctat

SEQ ID NO: 16 - DNA, 23 bp, synthetic construct
  source 1..23: mol_type = other DNA
  misc_feature 1..23: note = Primer
cctgtttggtaaggaaacaaagg

SEQ ID NO: 17 - DNA, 25 bp, synthetic construct
  source 1..25: mol_type = other DNA
  misc_feature 1..25: note = Primer
agctcttataatccttcttcccttt

SEQ ID NO: 18 - DNA, 20 bp, synthetic construct
  source 1..20: mol_type = other DNA
  misc_feature 1..20: note = Primer
taccgacgtcgtactgagaa

SEQ ID NO: 19 - DNA, 32 bp, synthetic construct
  source 1..32: mol_type = other DNA
  misc_feature 1..32: note = Primer
cccttttaattattttctctctgctatttttg

SEQ ID NO: 20 - DNA, 20 bp, synthetic construct
  source 1..20: mol_type = other DNA
  misc_feature 1..20: note = Primer
ttctccaattccatggacac

SEQ ID NO: 21 - DNA, 22 bp, synthetic construct
  source 1..22: mol_type = other DNA
  misc_feature 1..22: note = Primer
ccaagtagttatcgtgtgatcg

SEQ ID NO: 22 - DNA, 21 bp, synthetic construct
  source 1..21: mol_type = other DNA
  misc_feature 1..21: note = Primer
gtccaaagtcgtggatgaaga

SEQ ID NO: 23 - DNA, 21 bp, synthetic construct
  source 1..21: mol_type = other DNA
  misc_feature 1..21: note = Primer
tcccgattccaaatgggtatg

SEQ ID NO: 24 - DNA, 22 bp, synthetic construct
  source 1..22: mol_type = other DNA
  misc_feature 1..22: note = Primer
tcgtgcagtgaaagagaatagg

SEQ ID NO: 25 - DNA, 21 bp, synthetic construct
  source 1..21: mol_type = other DNA
  misc_feature 1..21: note = Primer
cattaccgttgcagcttgttt

SEQ ID NO: 26 - DNA, 22 bp, synthetic construct
  source 1..22: mol_type = other DNA
  misc_feature 1..22: note = Primer
ctgctccaacttctcaatacga

SEQ ID NO: 27 - DNA, 22 bp, synthetic construct
  source 1..22: mol_type = other DNA
  misc_feature 1..22: note = Primer
ctccacatttgctaggacaaga

SEQ ID NO: 28 - DNA, 23 bp, synthetic construct
  source 1..23: mol_type = other DNA
  misc_feature 1..23: note = Primer
acaacaatactgttactgctcca

SEQ ID NO: 29 - DNA, 17 bp, synthetic construct
  source 1..17: mol_type = other DNA
  misc_feature 1..17: note = Primer
ggacaagacccggcaac

SEQ ID NO: 30 - DNA, 8503 bp, Brassica napus
  source 1..8503: mol_type = unassigned DNA
atgagctcttataatctttcttcccttttcattatcttttctcttatacgtttcttgatctttgatctcactgttgccaggtcttgtagtagccattgtggaggaatccaaatcccatacccatttggaatcggggagggctgctacctcaataaatggtacgaaattaaatgtgatcataataactctctttcagtttcagggaagctcgtccctgttctttcagcaatcggcatggaagttgtgaatatctcttttccggatacactcgacacgtctggttctgaccgcgcaccatacgggtcagttcgcataaaatacccaataacttcaaaggggtgctccagcaacgaacaagagtctacatcacatctgaatttgacggggagccctttatacgttgctgttagtaatacactgatagcgattggttgcgacaacagggcatcgctgacaaatgtcgagccagtcctcgtgggatgtttctctagttgcctcgaaccaaaccctacagcttcagtacgggattatctagcaacagtcaattgtaatagtaagagtggctccgactacgggtattgtagcgaaagaagctatagaaatggtacaagctgcaacggtaatcaatgctgcgaggcaagcatgcccagtagtattcaacaggttgttggtgtcataatagatgctaactccaccacaacaagagggtgcaaagttgccttcttaacagatgaggcatactctttgttaaatgaaactgatccaaatttggttcttgctaaacgatattcaacagttgagttaggatggttcatccatacaacaaatctttcttccttcgtagacgctctaagatgtatgcctatgagagagtacctaaatctaagaagtagtgagcactcgtatagactgacttacggaataagttgtgtatgcgactataattcttattttcctaactatgcaagttgcagatgcaacagtggttacaggggaaacccatacattccggatggatgtaaaggttagtaagtatgtttatagttccttgtattcaagtcttctatcaaaagtaaactataagattatcatggttgattcaactttaattttttctttctttctaaaccagatattaatgaatgcactgaaaaagactcgaattgtggggacgacgccacttgtattaatttgctaggatcctatcaatgcattaatggaaatcattggccactttctataggtaaaccttgcaacttatatctcaaatagtttacccagaaattttttaaaaaaattatatctcaaatacagtgcgagataaattgtttattgggtaatatatcatcctactatcttcctatatatatatatattgggtaagatagtactatagtaggatgttatatgatgttatatataaatcatttacactaatttttcttatatgtcgatgaaattaagaattttttttaaaaaaatttgattggttgttgatacttaaattttatttaattaaaatctaaaataaataaaaaactaaccatagaatcaactaatctgggttaccagaaaacaattaaacattttaaacaaatcttaaatatatatgaatatgaattatttatgtatataaataattaaatataatagtttatattatataaacaatcttaaatattctactaataatgaatacaatacatcgtagctttcaacaaactaattaaccaagacctaataatattttgcatactcagtcactatatatatattctaaacaatgtgtcaactggatgcataacaatcaaatgtacaatttaaaaaaaaaacttactcattttcaatgacaactattgatattatgaatgaattatcacttggaaagaatgattaaagtatttgtgtcggtgttttaagtatttaaaatagaaattatgatcaattattaaataatactattaaccgatgaaaagcttgttatgatataaaattaaaaattgtaatatgatattgatacaagattggaattaatcctctctaaataaaattcacatatcatttgatcagaacaaattaaattggcgtaggaaaaggacgggagacataaataaattaaatgcatgagtcaagataataaaattttacactgataatagaaaaaagtatgtaaaagatttttgtcaactagtttgtggtaaaattggttattttatatttttattttatgtatttaattttttgtttgtttgtcaaaagacatatgtttttttcgtattattagaactgcgaaaaaatcagtttatgtgttttttacaacttagaaataaattaattaaggaagataggtacaattgatccactctaaaattaatatggtcaaagaagaaaaataataaaaatagaaagaaaagaaaagtatctacaaaataacaataggtactatttttatggtacaaaataaaattatgatgtatgataaagaagccctaataaatttttttttttaaatatgttattttgatgtaataaaaaattattgtaatctgacagtaatattaagtatatattttcaaaatgatgacattacattattttaaaatttagatattatttttatgttataaaattagaaattagttgattcaattttattaaaatatatttgttaagtaaatttaaagtaaatgaaaataatggctttaaaagaaagttttggtcaaatctaaattatagttataattatccttaattaaagtcaaatgactcagtgtttgataaaataatgtgattattggaaagagcttcaagatggatttgtttgctaactgatactacatttgttttatattaagtgtcactttagatttatacatacagattaagaaaatatttaactttacatattttttatataaaaatatcattatccgtacacctaaccatatttcaatcaatagaaaaataaaatagaaaataaagttaataaattatgtattgaatatttaaatgacacttattttacaacgaaagattatctatagaacgatacttaatatgaaacggaaggagtagtaatttgtgtgttgaatgcttaattaagcatgtgagataaagtattttgtaatttgacaaacatgtggaaactttacaactaaaataatataaggtttaaaagtgttctaaactattttttttgggttcattttttatgttttatattagtcatacatttatttatctaaacttttttttttgatctttatctatttagattaaactttttaacactatcactaccgttctaaaatgacatactagagcttttgattttcaaactatttgaaaattttattttttaagatttttgattattattaaaaaaattacatattataaacatacaatgtttatatccacgatatcatgaataaccacctaatatacatatatttataaaataaactggataatcaaaatctcactgttgtaattgaacttaaatatttatataatttttttagattgacataaaccaaacagaattgaaatagtgtaattattgttataagtcttatagagtgtagtcatgttcaatgaaagaatatatatatagatatatactattctacatatgatttgaataacaaatgatttgctttaagatttttaagccaacaagccaatgaatatgatttcaacatgtattttaaaattatattatattaaggaataacgatatttttagtttggattttcaaatacattaaatacatgcagaaccaataatctgcacttagtttttgtataaaattaatgaatagtctcatgttattttttatttctcttttctaaaatattaattagaaacatgtataatatatacttattttagaagttacaatatatattaattagagggtaataaaaacattaattaaaatatatcctgaaaacaatcgccgttagagctttacatatctcatatatatattaaaagagaagaattgtgatttaatgtttgacactatattggacacgtgacaacttcacatcgatttcaatttaagtatactgatgtatcgacctcatttaataattaatgcggtcacacactatttttttttaactccaccacagacaatttaatgtgtttacactaatttttcaatcagttcatttttagttccttcatttttaacatttgtattagtaaatttaaattatctctgtacagtactaaataatatcattttaaaagaaaaaactaaaaaattatttagatttgcatcatctaaattgctgaaatggtaattattttagaaactgaaagtttcgtatgttgccgaacttagccatggttgaggtttttgtacacgaatgcaataaataattatgactggttatgaattaaggaaattgaagtaaaaaataatacacacaaatttggatctttagtttagccaaatattgtccttctttacggagttttcatatcagaccaagaaatagaaacatgatcagattgctaaaaaaaacataaatcagaaaaacgttaaaaggcaaaaaagctcataaaatcgattgaaaaatagaattcagataccgactaaagagatcacattattttccaccgaactctttaacatttttcactcaattatatggtgttttccttatatttaccaataattttttttttataataagtacatgttattggttaaaaactatgtcagttttgaaacatgtccaacaataccatttttattggtattcttaaaatatggtccaataattatgtatgttaaataagcccataatatatttttacatttatttaaatttgctttatattattagaagaaagtaagtctgggtaaaatatatgatccaaagaaccgaactgaaatagatttgatagtaatactaaacacaaagtaaaactgattaagtactcatatggatataaaaaattaatattcaaattactagatccgaatcagatccaaactcaatattttggataccaaaaatatctaactcataattatatacgtaaataagttaattattataaatttatatctattagatattaaaaatattaaaatatcaaaaaattatcaacatagccccaagtaaatatataaaaataatagtaaatattcaaatgctgaaaatatttatttgttcttcatccaaatatttaaattgagctaatttttatgcaaatttaaatatttagtcttatgttattcaaatttttatgtttgtaatattttttttggatttggatgatttaagtatatttggatttttgttaaaaaatacataattaaaatggataccctacctcgaatccggaccgaaccctcaatgtttcaaaccaaatccaaacccaaagttataaatatttgaatacgatttaaatttctaactctgaaattagaaacccgaatagatcaactgaatctgaacgggtatatgaatgcccattcctagaaaaaactatacaacaaatcctttttgtacaacatacaataaataatctaataaaatattttattttgtgcgtaacacagattactcctaatacataaataattatgcaaactagtatatctatatataagaagaatgaagtaaggtaccttttaaaacaaagcaagagcaagcaaacatatatagtaatatatcacttatatattaattgaaaaacattacaacattggttgtagccacgtgtcatcactatgatgattcttagaatttttagaaaaattagtatactttttattaaactaaatatcaaattgatatgcattatacaggacaatattctcttttttttccttaaataaaatctatggaattacttatatgattagcatatatatgacaattaataattttgaataataaaaatttgataaaaaatttgtatttttcatctttttttagttttatattcttcaaaaaactaaacaatcacattaatcatttagaaatatttagatttttttaaattgttatattttgaatttttttaaacgtctataaattactaatattgttaaaagtatcacattgaaaaaatgtgattaatggtttaacttgttaacatttttgttataataagatacaaatgatcatacaatcatatgaataataaatttcatttaatagatattcatattaaatatatatcttaaatatgtattattgaaattattatatacaccatataaaatcataaatatgttaaattcaaaatttgcattgaaaaagtattgagactttaatattttatttttttaatttgcattcaatttttaaaaacaattataagttactaaaattatttaaaatctcacattgaaaattttgtgattaatggtttaatttttttttggtacaacaaatatacaaatgttaataaaattatatgagtagtaagcatcaattaataaatattcatattaaaatatattatatatctatgtcaatatcattaaaatttaattatatgacatataaaataatttttgtatgtatttaccaataaaaattattgtaaataaacaagagggattgttttgatttatgtgtagtccaatttaattatatatataatagttatttacttctcaattattaatatatttattatatcataatatgtaaataaatacaaaataaataataatatgttaaagtaattagtatactccctccgtttcagattatttgtcgttgtagagtaaaatttttgttttaaaataagtgtcattttataatttcaatacaaactttattggttttatatttaaaatctatttttcaattagttgaaatttggttaggtgtattggtaatgatgtttttatctagtaaatatacaaaattaaatgttttcttaatatgtgtgtcaaaatctaaaacgacaaataatatgaaacggatgaagtgtataatattctttccgcgctttcatctcgtgcaacgcagatctttacctagtatatatattaaatgtgaaaccaaatcaccacatattaaagatcaaacgtgcatgcttgcaagagaaatgtaaaagttataggggtcaattgacctccctccctacaccaatttaaaaatattgtagaagtcgactattgacaaatatttaatcattcactggagctagcgtgtcagaaaagtaaattttctgacaaaaagaaaagtaatcctttttccttttgttgggtttgtaggaattggttcaagtttgagctcattgatctttgttggtggaatatacgggttatacaaacttattagaaaacaaaggaggttgtcaaaccaaaagaagaagttcttcaagcgtaatggaggtttattgttgcagcaacaattaacttcaactgatggcaacattgaaaagacaatattgttcagctctagagagcttaagcaagccactgaaaactttagtttaactagagtacttggccaaggtggtcaaggtaccgtgtacaagggaatacttgttgatggtagagttgtagcagtaaaaaaatcaaaagtcgtggatgaagacaagttggaagagtttatcaatgaggttgtcattctctcccagatcaaccacagaaacattgtgaaacttttagggtgttgcctcgagacagatgtacctgttctcgtctatgagttcattcccaatggcaaccttttcgaacatcttcatgatgagttggttgatcaaacaatgactatttgggaactgcgtctccgcattgctatagatactgctgcagctctttcatatttgcattcagctgcatcatttccaatatatcacagagacatcaaatctacaaatataatgttggatgagagatatggagctaaaatatctgattttggcacttcaagaacagtaacagtggatcatacacaccttacaaccgtcgtttcaggtacggtgggatatctggatcctgagtatttccagtctaaccagttcacagataaaagtgatgtttacagctttggagtggtgcttgtcgagcttatcacaggagaaaaatctgtttctttcttgcggtctcaagaaaataggatattagcaacttatttcattcttgcaatgaaagagaacagactatttgacattatcgatgcacggatcagagatagttgcatgtggaatcaagtgactgcaatggcaaatattgcaaggaagtgtttgagtcggaaagggaggcaacggccaagtatgagagaagtgtccatggaattggagaaaattcgtgcgtcttcggtggatatggaactacatgagtatggtagtgaaaatgaggacgaccatgaagatcgagttgtggatgctaacatagagttacaatcatggaacaacgttgctgttactgccccaacttctcaatacgacgtgggtacatcatctttgtcaatgtcagatgttgagcctctatttccttttcaaacacggtaa

SEQ ID NO: 31 - DNA, 2379 bp, Brassica napus
  source 1..2379: mol_type = unassigned DNA
atgagctcttataatctttcttcccttttcattatcttttctcttatacgtttcttgatctttgatctcactgttgccaggtcttgtagtagccattgtggaggaatccaaatcccatacccatttggaatcggggagggctgctacctcaataaatggtacgaaattaaatgtgatcataataactctctttcagtttcagggaagctcgtccctgttctttcagcaatcggcatggaagttgtgaatatctcttttccggatacactcgacacgtctggttctgaccgcgcaccatacgggtcagttcgcataaaatacccaataacttcaaaggggtgctccagcaacgaacaagagtctacatcacatctgaatttgacggggagccctttatacgttgctgttagtaatacactgatagcgattggttgcgacaacagggcatcgctgacaaatgtcgagccagtcctcgtgggatgtttctctagttgcctcgaaccaaaccctacagcttcagtacgggattatctagcaacagtcaattgtaatagtaagagtggctccgactacgggtattgtagcgaaagaagctatagaaatggtacaagctgcaacggtaatcaatgctgcgaggcaagcatgcccagtagtattcaacaggttgttggtgtcataatagatgctaactccaccacaacaagagggtgcaaagttgccttcttaacagatgaggcatactctttgttaaatgaaactgatccaaatttggttcttgctaaacgatattcaacagttgagttaggatggttcatccatacaacaaatctttcttccttcgtagacgctctaagatgtatgcctatgagagagtacctaaatctaagaagtagtgagcactcgtatagactgacttacggaataagttgtgtatgcgactataattcttattttcctaactatgcaagttgcagatgcaacagtggttacaggggaaacccatacattccggatggatgtaaagatattaatgaatgcactgaaaaagactcgaattgtggggacgacgccacttgtattaatttgctaggatcctatcaatgcattaatggaaatcattggccactttctataggaattggttcaagtttgagctcattgatctttgttggtggaatatacgggttatacaaacttattagaaaacaaaggaggttgtcaaaccaaaagaagaagttcttcaagcgtaatggaggtttattgttgcagcaacaattaacttcaactgatggcaacattgaaaagacaatattgttcagctctagagagcttaagcaagccactgaaaactttagtttaactagagtacttggccaaggtggtcaaggtaccgtgtacaagggaatacttgttgatggtagagttgtagcagtaaaaaaatcaaaagtcgtggatgaagacaagttggaagagtttatcaatgaggttgtcattctctcccagatcaaccacagaaacattgtgaaacttttagggtgttgcctcgagacagatgtacctgttctcgtctatgagttcattcccaatggcaaccttttcgaacatcttcatgatgagttggttgatcaaacaatgactatttgggaactgcgtctccgcattgctatagatactgctgcagctctttcatatttgcattcagctgcatcatttccaatatatcacagagacatcaaatctacaaatataatgttggatgagagatatggagctaaaatatctgattttggcacttcaagaacagtaacagtggatcatacacaccttacaaccgtcgtttcaggtacggtgggatatctggatcctgagtatttccagtctaaccagttcacagataaaagtgatgtttacagctttggagtggtgcttgtcgagcttatcacaggagaaaaatctgtttctttcttgcggtctcaagaaaataggatattagcaacttatttcattcttgcaatgaaagagaacagactatttgacattatcgatgcacggatcagagatagttgcatgtggaatcaagtgactgcaatggcaaatattgcaaggaagtgtttgagtcggaaagggaggcaacggccaagtatgagagaagtgtccatggaattggagaaaattcgtgcgtcttcggtggatatggaactacatgagtatggtagtgaaaatgaggacgaccatgaagatcgagttgtggatgctaacatagagttacaatcatggaacaacgttgctgttactgccccaacttctcaatacgacgtgggtacatcatctttgtcaatgtcagatgttgagcctctatttccttttcaaacacggtaa

SEQ ID NO: 32 - protein, 792 aa, Brassica napus
MSSYNLSSLFIIFSLIRFLIFDLTVARSCSSHCGGIQIPYPFGIGEGCYLNKWYEIKCDHNNSLSVSGKLVPVLSAIGMEVVNISFPDTLDTSGSDRAPYGSVRIKYPITSKGCSSNEQESTSHLNLTGSPLYVAVSNTLIAIGCDNRASLTNVEPVLVGCFSSCLEPNPTASVRDYLATVNCNSKSGSDYGYCSERSYRNGTSCNGNQCCEASMPSSIQQVVGVIIDANSTTTRGCKVAFLTDEAYSLLNETDPNLVLAKRYSTVELGWFIHTTNLSSFVDALRCMPMREYLNLRSSEHSYRLTYGISCVCDYNSYFPNYASCRCNSGYRGNPYIPDGCKDINECTEKDSNCGDDATCINLLGSYQCINGNHWPLSIGIGSSLSSLIFVGGIYGLYKLIRKQRRLSNQKKKFFKRNGGLLLQQQLTSTDGNIEKTILFSSRELKQATENFSLTRVLGQGGQGTVYKGILVDGRVVAVKKSKVVDEDKLEEFINEVVILSQINHRNIVKLLGCCLETDVPVLVYEFIPNGNLFEHLHDELVDQTMTIWELRLRIAIDTAAALSYLHSAASFPIYHRDIKSTNIMLDERYGAKISDFGTSRTVTVDHTHLTTVVSGTVGYLDPEYFQSNQFTDKSDVYSFGVVLVELITGEKSVSFLRSQENRILATYFILAMKENRLFDIIDARIRDSCMWNQVTAMANIARKCLSRKGRQRPSMREVSMELEKIRASSVDMELHEYGSENEDDHEDRVVDANIELQSWNNVAVTAPTSQYDVGTSSLSMSDVEPLFPFQTR

SEQ ID NO: 33 - DNA, 8509 bp, Brassica napus
  source 1..8509: mol_type = unassigned DNA
atgagctcttataatctttcttcccttttcattatcttttctcttatatttttcttgatatttgatctcactgttgccaggtcttgtagtagccattgcggaggaatccacatcccgtacccatttggaatcggggagggctgctacctcaacaaatggtacgaaattaaatgtgatgataataactctatttcagtttcagggaagcctgtccctgttctttcagcaatcggcatggaagttgtgaatatctcttttccggatacactcgacacgtctggttctgaccgcgcaccatacgggtcagttcgcataaaatacccaataacttcaaaggggtgctccagcaacgaacaagagtctacatcacatctgaatttgacggggagccctttatacgttgctgttagtaatacactgatagcgattggttgcgacaacagggcatcgctgacaaatgtcgagccagtcctcgtgggatgtttctctagttgcctcgaaccaaaccctacagcttcagtacgggattatctagcaacagtcaattgtaatagtaagagtggctccgactacgggtattgtagcgaaagaagctatagaaatggtacaagctgcaacggtaatcaatgctgcgaggcaagcatgcccagtagtattcaacaggttgttggtgtcataatagatgctaactccaccacaacaagagggtgcaaagttgccttcttaacagatgaggcatactctttgttaaatgaaactgatccaaatttggttcttgctaaacgatattcaacagttgagttaggatggttcatccatacaacaaatctttcttccttcgtagactctctaagatgtatgcctatgagagagtacctaaatctaagaagtagtgagcactcgtatagactgacttacggaataagttgtgtatgcgactataattcttattttcctaactatgcaagttgcagatgcaacagtggttacaggggaaacccatacattccggatggatgtaaaggttagtaagtatgtttatagttccttgtattcaagtcttctatcaaaagtaaactataagattatcatggttgattcaactttaattttttctttctttctaaaccagatattaatgaatgcactgaaaaagactcgaattgtggggacgacgccacttgtattaatttgctaggatcctatcaatgcattaatggaaatcattggccactttctataggtaaaccttgcaacttatatctcaaatagtttacccagaaattttttaaaaaaattatatctcaaatacagtgcgagataaattgtttattgggtaatataacatcctactatcttcctatatatatatatatattgggtaagatagtactatagtaggatgttatatgatgttatatataaatcatttacactaatttttcttatatgtcgatgaaattaagaattttttttaaaaaaatttgattggttgttgatacttaaattttatttaattaaaatctaaaataaataaaaaactaaccgtagaatcaactaatctgggttaccagaaaacaattaaacattttaaacaaatcttaaatatatatgaatatgaattatttatgtatataaataattaaatataatagtttatattatataaacaatcttaaatattctactaataatgaatacaatacatcgtagctttcaacaaactaattaaccaagacctaataatattttgcatactcagtcactatatatatattctaaacaatgtgtcaactggatgcataacaatcaaatgtacaatttaaaaaaaaaacttactcattttcaatgacaactattgatattatgaatgaattatcacttggaaagaatgattaaagtatttgtgtcggtgttttaagtatttaaaatagaaattatgatcaattattaaataatactattaaccgatgaaaagcttgttatgatataaaattaaaaattgtaatatgatattgatacaagattggaattaatcctctctaaataaaattcacatatcatttgatcagaacaaattaaattggcataggaaaaggacgggagacataaataaattaaatgcatgagtcaagataataaaattttacactgataatagaaaaaagtatgtaaaagatttttgtcaactagtttgtggtaaaattggttattttatatttttattttatgtatttaattttttgtttgtttgtcaaaagacatatgtttttttcgtattattagaactgcgaaaaaatcagtttatgtgttttttacaacttagaaataaattaattaaggaagataggtacaattgatccactctaaaattaatatggtcaaagaagaaaaataataaaaatagaaagaaaagaaaagtatctacaaaataacaataggtactatttttatggtacaaaataaaattatgatgtatgataaagaagccctaataaatttttttttttaaatatgttattttgatgtaataaaaaattattgtaatctgacagtaatattaagtatatattttcaaaatgatgacattacattattttaaaatttaaatattatttttatgttataaaattagaaattagttgattcaattttattaaaatatatttgttaagtaaatttaaagtaaatgaaaataatggctttaaaagaaagttttggtcaaatctaaattatagttataattatccttaattaaagtcaaatgactcagtgtttgataaaataatgtgattattggaaagagcttcaagatggatttgtttgctaactgatactacatttgttttatattaagtgtcactttagatttatacatacagattaagaaaatatttaactttacatattttttatataaaaatatcattatccgtacacctaaccatatttcaatcaatagaaaaataaaatagaaaataaagttaataaattatgtattgaatatttaaatgacacttattttacaacgaaagattatctatagaacgatacttaatatgaaacggaaggagtagtaatttgtgtgttgaatgcttaattaagcatgtgagataaagtattttgtaatttgacaaacatgtggaaactttacaactaaaataatataaggtttaaaagtgttctaaactattttttttgggttcattttttatgttttatattagtcatacatttatttatctaaactttttttttgatctttatctatttagattaaactttttaacactatcactaccgttctaaaatgacatactagagcttttgattttcaaactatttgaaaattttattttttaagatttttgattattattaaaaaaattacatattataaacatacaatgtttatatccacgatatcatgaataaccacctaatatacatatatttataaaataaactggataatcaaaatctcactgttgtaattgaacttaaatatttatataatttttttagattgacataaaccaaacagaattgaaatagtgtaattattgttataagtcttatagagtgtagtcatgttcaatgaaagaatatatatatagatatatactattctacatatgatttgaataacaaatgatttgctttaagatttttaagccaacaagccaatgaatatgatttcaacgtgtattttaaaattatattatattaaggaataacgatatttttagtttggattttcaaatacattaaatacatgcataaccaataatctgcacttagtttttgtataaaattaatgaatagtctcatgttattttttatttctcttttctaaaatattaattagaaacatgtataatatataattattttagaagttacaatatatattaattagagggtaataaaaatattaattaaaatatatcctgaaaacaatcgccgttagagctttacatatctcatatatatattaaaagagaagaattgtgatttaatgtttgacactatattggacacgtgacaacttcacatcgatttcaatttaagtatactgatgtatcgacctcatttaataattaatgcggtcacacactatttttttttaactccaccacagacaatttaatgtgtttacactaatttttcaatcagttcatttttagttccttcatttttaacatttgtattagtaaatttaaattatctctgtacagtactaaataatatcattttaaaagaaaaaactaaaaaattatttagatttgcatcatctaaattgctgaaatggtaattattttagaaactgaaagtttcgtatgttgccgaacttagccatggttgaggtttttgtacacgaatgcaataaataattatgactggttatgaattaaggaaattgaagtaaaaaataatacacacaaatttggatctttagtttagccaaatattgtccttctttacggagttttcatatcagaccaagaaatagaaacatgatcagattgctaaaaaaaacataaatcagaaaaacgttaaaaggcaaaaaagctcttaaaatcgattgaaaaatagaattcagataccgactaaagagatcacattattttccaccgaactctttaacatttttcactcaattatatagtgttttccttatatttaccaataattttttttttataataagtacatgttattggttaaaaactatgtcagttttgaaacatgtccaacaataccatttttattggtattcttaaaatatggtccaataattatgtatgttaaataagcccataatatatttttacatttatttaaatttgctttatattattagaagaaagtaagtctgggtaaaatatatgatccaaagaaccgaactgaaatagatttgatagtaatactaaacacaaagtaaaactgattaagtactcatatggatataaaaaattaatattcaaattactagatccgaatcagatccaaactcaatattttggataccaaaaatatctaactcataattatatacgtaaataagttaattattataaatttatatctattagatattaaaaatattaaaatatcaaaaaattatcaacatagccccaagtaaatatataaaaataatagtaaatattcaaatgctgaaaatatttatttgttcttcatccaaatatttaaattgagctaatttttatgcaaatttaaatatttagtcttatgttattcaaatttttatgtttgtaatatttttttttggatttggatgatttaagtatatttggatttttgttaaaaaatacataattaaaatggataccctacctcgaatccggaccgaaccctcaatgtttcaaaccaaatccaaacccaaagttataaatatttgaatacgatttaaatttctaactctgaaattagaaacccgaatagatcaactgaatctgaacgggtatatgaatgcccattcctagaaaaaactatacaacaaatcctttttgtacaacatacaataaataatctaataaaatattttattttgtgcgtaacacagattactcctaatacataaataattatgcaaactagtatatctatatataagaagaatgaagtaaggtaccttttaaaacaaagcaagagcaagcaaacatatatagtaatatatcacttatatattaattgaaaaacattacaacattggctgtagccacgtgtcatcactatgatgattcttagaatttttagaaaaattagtatactttttattaaactaaatatcaaattgatatgcattatacaggacaatattctcttttttttccttaaataaaatctatggaattacttatatgattagcatatatatgacaattaataattttgaataataaaaatttgataaaaaatttgtatttttcatctttttttagttttatattcttcaaaaaactaaacaatcacattaatcatttagaaatatttagatttttttaaattgttatattttgaatttttttaaacgtctataaattactaatattgttaaaagtatcacattgaaaaaatgtgattaatggtttaacttgttaacatttttgttataataagatacaaatgatcatacaatcatatgaataataaatttcatttaatagatattcatattaaatatatatcttaaatatgtattattgaaattattatatacaccatataaaatcataaatatgttaaattcaaaatttgcattgaaaaagtattgagactttaatattttatttttttaatttgcattcaatttttaaaaacaattataagttactaaaattatttaaaatctcacattgaaaattttgtgattaatggtttaattttttttttggtacaacaaatatacaaatgttaataaaattatatgagtagtaagcattaattaataaatattcatattaaaatatattatatatctatgtcaatatcattaaaatttaattatatgacatataaaataatttttgtatgtatttaccaataaaaattattgtaaataaacaagagggattgttttgatttatgtgtagtccaatttaattatatatataatagttatttacttctcaattattaatatatttattatatcataatatgtaaataaatacaaaataaataataatatgttaaagtaattagtatactccctccgtttcagattatttgtcgttgtagagtaaaatttttgttttaaaataagtgtcattttataatttcaatacaaactttattggttttatatttaaaatctatttttcaattagttgaaatttggttaggtgtattggtaatgatgtttttatctagtaaatatacaaaattaaatgttttcttaatatgtgtgtcaaaatctaaaacgacaaataatatgaaacggatggagtgtataatattctttccgcgctttcatctcgtgcaacgcagatctttacctagtatatatattaaatgtgaaaccaaatcaccacatattaaagatcaaacgtgcatgcttgcaagagaaattttgtaaaagttataggggtcaattgacctccctccctacaccaatttaaaaatattgtagaagtcgactattgacaaatatttaatcattcactggagctagcgtgtcagaaaagtaaattttctgacaaaaagaaaagtaatcctttttccttttgttgggtttgtaggaattggttcaagtttgagctcattgatctttgttggtggaatatacgggttatacaaacttattagaaaacaaaggaggttgtcaaaccaaaagaagaagttcttcaagcgtaatggaggtttattgttgcagcaacaattaacttcaactgatggcaacattgaaaagacaatattgttcagctctagagagcttaagcaagccactgaaaactttagtttaactagagtacttggccaaggtggtcaaggtaccgtgtacaagggaatacttgttgatggtagagttgtagcagtaaaaaaatcaaaagtcgtggatgaagacaagttggaagagtttatcaatgaggttgtcattctctcccagatcaaccacagaaacattgtgaaacttttagggtgttgcctcgagacagatgtacctgttctcgtctatgagttcattcccaatggcaaccttttcgaacatcttcatgatgagttggttgatcaaacaatgactatttgggaactgcgtctccgcattgctatagatactgctgcagctctttcatatttgcattcagctgcatcatttccaatatatcacagagacatcaaatctacaaatataatgttggatgagagatatggagctaaaatatctgattttggcacttcaagaacagtaacagtggatcatacacaccttacaaccgtcgtttcaggtacggtgggatatctggatcctgagtatttccagtctaaccagttcacagataaaagtgatgtttacagctttggagtggtgcttgtcgagcttatcacaggagaaaaatctctttctttcttgcggtctcaagaaaataggatattagcaacttatttcattcttgcaatgaaagagaacagactatttgacattatcgatgcacggatcagagatagttgcatgtggaatcaagtgactgcaatggcaaatattgcaaggaagtgtttgagtcggaaagggaggcaacggccaagtatgagagaagtgtccatggaattggagaaaattcgtgcgtcttcggtggatatggaactacatgagtatggtagtgaaaatgaggacgaccatgaagatcgagttgtggatgctaacatagagttacaatcatggaacaacgttgctgttactgccccaacttctcaatacgacgtgggtacatcatctttgtcaatgtcagatgttgagcctctatttccttttcaaacacggtaa

SEQ ID NO: 34 - DNA, 2379 bp, Brassica napus
  source 1..2379: mol_type = unassigned DNA
atgagctcttataatctttcttcccttttcattatcttttctcttatatttttcttgatatttgatctcactgttgccaggtcttgtagtagccattgcggaggaatccacatcccgtacccatttggaatcggggagggctgctacctcaacaaatggtacgaaattaaatgtgatgataataactctatttcagtttcagggaagcctgtccctgttctttcagcaatcggcatggaagttgtgaatatctcttttccggatacactcgacacgtctggttctgaccgcgcaccatacgggtcagttcgcataaaatacccaataacttcaaaggggtgctccagcaacgaacaagagtctacatcacatctgaatttgacggggagccctttatacgttgctgttagtaatacactgatagcgattggttgcgacaacagggcatcgctgacaaatgtcgagccagtcctcgtgggatgtttctctagttgcctcgaaccaaaccctacagcttcagtacgggattatctagcaacagtcaattgtaatagtaagagtggctccgactacgggtattgtagcgaaagaagctatagaaatggtacaagctgcaacggtaatcaatgctgcgaggcaagcatgcccagtagtattcaacaggttgttggtgtcataatagatgctaactccaccacaacaagagggtgcaaagttgccttcttaacagatgaggcatactctttgttaaatgaaactgatccaaatttggttcttgctaaacgatattcaacagttgagttaggatggttcatccatacaacaaatctttcttccttcgtagactctctaagatgtatgcctatgagagagtacctaaatctaagaagtagtgagcactcgtatagactgacttacggaataagttgtgtatgcgactataattcttattttcctaactatgcaagttgcagatgcaacagtggttacaggggaaacccatacattccggatggatgtaaagatattaatgaatgcactgaaaaagactcgaattgtggggacgacgccacttgtattaatttgctaggatcctatcaatgcattaatggaaatcattggccactttctataggaattggttcaagtttgagctcattgatctttgttggtggaatatacgggttatacaaacttattagaaaacaaaggaggttgtcaaaccaaaagaagaagttcttcaagcgtaatggaggtttattgttgcagcaacaattaacttcaactgatggcaacattgaaaagacaatattgttcagctctagagagcttaagcaagccactgaaaactttagtttaactagagtacttggccaaggtggtcaaggtaccgtgtacaagggaatacttgttgatggtagagttgtagcagtaaaaaaatcaaaagtcgtggatgaagacaagttggaagagtttatcaatgaggttgtcattctctcccagatcaaccacagaaacattgtgaaacttttagggtgttgcctcgagacagatgtacctgttctcgtctatgagttcattcccaatggcaaccttttcgaacatcttcatgatgagttggttgatcaaacaatgactatttgggaactgcgtctccgcattgctatagatactgctgcagctctttcatatttgcattcagctgcatcatttccaatatatcacagagacatcaaatctacaaatataatgttggatgagagatatggagctaaaatatctgattttggcacttcaagaacagtaacagtggatcatacacaccttacaaccgtcgtttcaggtacggtgggatatctggatcctgagtatttccagtctaaccagttcacagataaaagtgatgtttacagctttggagtggtgcttgtcgagcttatcacaggagaaaaatctctttctttcttgcggtctcaagaaaataggatattagcaacttatttcattcttgcaatgaaagagaacagactatttgacattatcgatgcacggatcagagatagttgcatgtggaatcaagtgactgcaatggcaaatattgcaaggaagtgtttgagtcggaaagggaggcaacggccaagtatgagagaagtgtccatggaattggagaaaattcgtgcgtcttcggtggatatggaactacatgagtatggtagtgaaaatgaggacgaccatgaagatcgagttgtggatgctaacatagagttacaatcatggaacaacgttgctgttactgccccaacttctcaatacgacgtgggtacatcatctttgtcaatgtcagatgttgagcctctatttccttttcaaacacggtaa

SEQ ID NO: 35 - protein, 792 aa, Brassica napus
MSSYNLSSLFIIFSLIFFLIFDLTVARSCSSHCGGIHIPYPFGIGEGCYLNKWYEIKCDDNNSISVSGKPVPVLSAIGMEVVNISFPDTLDTSGSDRAPYGSVRIKYPITSKGCSSNEQESTSHLNLTGSPLYVAVSNTLIAIGCDNRASLTNVEPVLVGCFSSCLEPNPTASVRDYLATVNCNSKSGSDYGYCSERSYRNGTSCNGNQCCEASMPSSIQQVVGVIIDANSTTTRGCKVAFLTDEAYSLLNETDPNLVLAKRYSTVELGWFIHTTNLSSFVDSLRCMPMREYLNLRSSEHSYRLTYGISCVCDYNSYFPNYASCRCNSGYRGNPYIPDGCKDINECTEKDSNCGDDATCINLLGSYQCINGNHWPLSIGIGSSLSSLIFVGGIYGLYKLIRKQRRLSNQKKKFFKRNGGLLLQQQLTSTDGNIEKTILFSSRELKQATENFSLTRVLGQGGQGTVYKGILVDGRVVAVKKSKVVDEDKLEEFINEVVILSQINHRNIVKLLGCCLETDVPVLVYEFIPNGNLFEHLHDELVDQTMTIWELRLRIAIDTAAALSYLHSAASFPIYHRDIKSTNIMLDERYGAKISDFGTSRTVTVDHTHLTTVVSGTVGYLDPEYFQSNQFTDKSDVYSFGVVLVELITGEKSLSFLRSQENRILATYFILAMKENRLFDIIDARIRDSCMWNQVTAMANIARKCLSRKGRQRPSMREVSMELEKIRASSVDMELHEYGSENEDDHEDRVVDANIELQSWNNVAVTAPTSQYDVGTSSLSMSDVEPLFPFQTR

SEQ ID NO: 36 - DNA, 2641 bp, Brassica napus
  source 1..2641: mol_type = unassigned DNA
atgagctcttataatctttcttcccttttcattatcttttctcttatatttttcttgatatttgatctcactgttgccaggtcttgtagtagccattgcggaggaatccacatcccgtacccatttggaatcggggagggctgctacctcaacaaatggtatgaaattaaatgtgatcataatacctctctttcagtttcagggaagcccgtccctgttctttcactgatcggcaaggaagttgtgaatatctctcttccggatatactcctctcgtatagttcttatcgcctgccacatgggaaagttcaaataagaaacccaatgacatcaaaggggtgctccagcaacgaacaagagtctacatcacttctgaactttacggggagccctttctacgttagtgatagtaatacactgatagctattggttgcaacaacagggcgtccttgacaaatgtcgagccaagcaccgtgggatgcttctccagttgcctcccaacaaatcgtactgcttcacaagattatctagagacagtcaattgtaatactaagagttactatgctgaagaaagctacgggtattgtcgtgagagaagatatataaatgaaacaagctgcaacggtaatcggtgttgcaaggcaagcatgcccggtagtattcaacaggtggttggtgtcacaatagatgataaccctaccacaagaggagggtgtaaagttgccttcttaacagatgatcaggcatactctttattagacggatctgatccaaatctggttcttgctaaacgatattcaacagttgagttaggatggttcatccatacaacgaatctttcttccttcgtagactctctaagatgtatgcctatgagagagtacctaaatctaagaaatagtgaacactatagcctgacttacggaataagttgtgcatgcgattataattcttcttttcctaatgatgcaagttgcagatgcaacaatggttacaagggaaacccatacattccgggtggatgtaaaggttagtaagtttgttaatagttcatattttttattcaagtcatctatatatgattatgatcaaattaactttaagatcatcatggttgatttcagtttcatatttttctttctaaaccagattttaatgaatgtgctgaaaaacgctattcagacaactgtgtgggaggcacttgtgtcgattttcaaggacactatgaatgtgtgtatagcaatcaccggcttgctataggtaaaccgtgcaactttctatatcaaattcattagcacccacttaaagatattgtataaaggcgatcttcatttattaaatcattcacccaaagccgtgcaagaaaatgaaatttggttttcgttattttttgcagggttcggttcaagtttcggctcattgatctttattgttggaatatacgggttatacagatttattagaaaacagaggaggttaaaccaaaagaagaagttcttcaagcgtaacggaggcttgttgttgcaacaacaattaacttcaacccaaggcaacgttgaaaagacaagagtgttcagctcgagagagctggaaaaggccaccgaaaacttcagtttaactagaatacttggccaaggcggtcaaggaaccgtctacaaaggaatgcttgttgatggaagaattgtagcagtgaaaaagtctaaagtggtggatgaagacaagttggaggagttcatcaatgaggttgtcattctctcccagatcaaccacagaaacatcgtcaaacttttagggtgttgcctcgagacagatgtccctgttctagtctacgagtttattcccaatggcaaccttttcgaacatcttcatgaagatgagttagatgatcacacgatgactacgtgggaagtgcgtctccgcattgctatagatattgcaagtgctctttcgtatttgcattcagccgcatcatcgccaatatatcatagggacgtcaagtctacaaacataatgttggatgagaagtatcgagctaaagtgtctgattttggcacttcaagaacggtaacggtggatcatacgcatcttacaaccgttgtttcaggtacggtggggtatgtggatcctgagtatttccagtctagccagttcacggataaaagcgatgtttacagctttggggtggtgcttgcggagcttatcaccggagaaaaatcagtttctttcttgcggtctcaagaaaataggacattggcaacttatttctttcttgcgatgaaagagaataggctgcttgacattatcgatgcacggatcagagatggttgcaagctgaatcaagtgactgcaacggcaaatcttgcaaggaagtgtttgaatctaaaagggaggaaacggccaagtatgcgagaagtgtccatggaattggagaaaattcgtgggtcttcggtggatatggaactacatgagtgtggtagtgaaaacgaggaagagaatgaagaacaaattgtgggtgctaacatagagttacaatcatggaacaacactgccgttactgctccaacttctcaatacgacgtcggtacatcttctttgccaatgtcagatgttgagcctctgtttccttaccaaacacggtaa

SEQ ID NO: 37 - DNA, 2385 bp, Brassica napus
  source 1..2385: mol_type = unassigned DNA
atgagctcttataatctttcttcccttttcattatcttttctcttatatttttcttgatatttgatctcactgttgccaggtcttgtagtagccattgcggaggaatccacatcccgtacccatttggaatcggggagggctgctacctcaacaaatggtatgaaattaaatgtgatcataatacctctctttcagtttcagggaagcccgtccctgttctttcactgatcggcaaggaagttgtgaatatctctcttccggatatactcctctcgtatagttcttatcgcctgccacatgggaaagttcaaataagaaacccaatgacatcaaaggggtgctccagcaacgaacaagagtctacatcacttctgaactttacggggagccctttctacgttagtgatagtaatacactgatagctattggttgcaacaacagggcgtccttgacaaatgtcgagccaagcaccgtgggatgcttctccagttgcctcccaacaaatcgtactgcttcacaagattatctagagacagtcaattgtaatactaagagttactatgctgaagaaagctacgggtattgtcgtgagagaagatatataaatgaaacaagctgcaacggtaatcggtgttgcaaggcaagcatgcccggtagtattcaacaggtggttggtgtcacaatagatgataaccctaccacaagaggagggtgtaaagttgccttcttaacagatgatcaggcatactctttattagacggatctgatccaaatctggttcttgctaaacgatattcaacagttgagttaggatggttcatccatacaacgaatctttcttccttcgtagactctctaagatgtatgcctatgagagagtacctaaatctaagaaatagtgaacactatagcctgacttacggaataagttgtgcatgcgattataattcttcttttcctaatgatgcaagttgcagatgcaacaatggttacaagggaaacccatacattccgggtggatgtaaagattttaatgaatgtgctgaaaaacgctattcagacaactgtgtgggaggcacttgtgtcgattttcaaggacactatgaatgtgtgtatagcaatcaccggcttgctatagggttcggttcaagtttcggctcattgatctttattgttggaatatacgggttatacagatttattagaaaacagaggaggttaaaccaaaagaagaagttcttcaagcgtaacggaggcttgttgttgcaacaacaattaacttcaacccaaggcaacgttgaaaagacaagagtgttcagctcgagagagctggaaaaggccaccgaaaacttcagtttaactagaatacttggccaaggcggtcaaggaaccgtctacaaaggaatgcttgttgatggaagaattgtagcagtgaaaaagtctaaagtggtggatgaagacaagttggaggagttcatcaatgaggttgtcattctctcccagatcaaccacagaaacatcgtcaaacttttagggtgttgcctcgagacagatgtccctgttctagtctacgagtttattcccaatggcaaccttttcgaacatcttcatgaagatgagttagatgatcacacgatgactacgtgggaagtgcgtctccgcattgctatagatattgcaagtgctctttcgtatttgcattcagccgcatcatcgccaatatatcatagggacgtcaagtctacaaacataatgttggatgagaagtatcgagctaaagtgtctgattttggcacttcaagaacggtaacggtggatcatacgcatcttacaaccgttgtttcaggtacggtggggtatgtggatcctgagtatttccagtctagccagttcacggataaaagcgatgtttacagctttggggtggtgcttgcggagcttatcaccggagaaaaatcagtttctttcttgcggtctcaagaaaataggacattggcaacttatttctttcttgcgatgaaagagaataggctgcttgacattatcgatgcacggatcagagatggttgcaagctgaatcaagtgactgcaacggcaaatcttgcaaggaagtgtttgaatctaaaagggaggaaacggccaagtatgcgagaagtgtccatggaattggagaaaattcgtgggtcttcggtggatatggaactacatgagtgtggtagtgaaaacgaggaagagaatgaagaacaaattgtgggtgctaacatagagttacaatcatggaacaacactgccgttactgctccaacttctcaatacgacgtcggtacatcttctttgccaatgtcagatgttgagcctctgtttccttaccaaacacggtaa

SEQ ID NO: 38 - protein, 794 aa, Brassica napus
MSSYNLSSLFIIFSLIFFLIFDLTVARSCSSHCGGIHIPYPFGIGEGCYLNKWYEIKCDHNTSLSVSGKPVPVLSLIGKEVVNISLPDILLSYSSYRLPHGKVQIRNPMTSKGCSSNEQESTSLLNFTGSPFYVSDSNTLIAIGCNNRASLTNVEPSTVGCFSSCLPTNRTASQDYLETVNCNTKSYYAEESYGYCRERRYINETSCNGNRCCKASMPGSIQQVVGVTIDDNPTTRGGCKVAFLTDDQAYSLLDGSDPNLVLAKRYSTVELGWFIHTTNLSSFVDSLRCMPMREYLNLRNSEHYSLTYGISCACDYNSSFPNDASCRCNNGYKGNPYIPGGCKDFNECAEKRYSDNCVGGTCVDFQGHYECVYSNHRLAIGFGSSFGSLIFIVGIYGLYRFIRKQRRLNQKKKFFKRNGGLLLQQQLTSTQGNVEKTRVFSSRELEKATENFSLTRILGQGGQGTVYKGMLVDGRIVAVKKSKVVDEDKLEEFINEVVILSQINHRNIVKLLGCCLETDVPVLVYEFIPNGNLFEHLHEDELDDHTMTTWEVRLRIAIDIASALSYLHSAASSPIYHRDVKSTNIMLDEKYRAKVSDFGTSRTVTVDHTHLTTVVSGTVGYVDPEYFQSSQFTDKSDVYSFGVVLAELITGEKSVSFLRSQENRTLATYFFLAMKENRLLDIIDARIRDGCKLNQVTATANLARKCLNLKGRKRPSMREVSMELEKIRGSSVDMELHECGSENEEENEEQIVGANIELQSWNNTAVTAPTSQYDVGTSSLPMSDVEPLFPYQTR

SEQ ID NO: 39 - DNA, 794 bp, Leptosphaeria maculans
  source 1..794: mol_type = unassigned DNA
aaggattatatatatatatatccctatcccttttatttttatatcattgtatatctaacatttctaacaacatcgtgctattattacaaatatgttaaaacctacaaaggtgatccaaatcttgtttctattatttaccgcattttttgcacgaacgtgcgctcttgaatgccacgctgttgctttctcttctgatcaccaattttctcttggtcgtaatgaggactgtaacttatactgcagcaaaaattcaatgcttagcatcttttcaagggttctgcctttagacgacccctgccaaggggatgatggatttagtagtcttacagccacgatcgatcaaataagttgtgatacaggatgtcaatgccgttgctctgttcacgccactgcttggcgagtccacaagtctggcaagcgatattcaagaagaactggctgggtatcttgcaatctagaagattatgttgctaggataactgggaggccttttataccagttaatggagcactacacgaatatttctcccgcggatttgtgtccaaagacgaggtacactgcgatcatcagtagtaagtagatcatatattccagatactacctacgtatagtactaattatcccaaaggagtatagttattgtgccgtggcaccctgggttcggtcgaaaacaatatctatcctaagttcaaataggtagaaatctgactcagttggaaaacagtatcaaagaggggaggattataggatcgaacgttaggcagcataatacgtaatgctttaacacatctta

SEQ ID NO: 40 - DNA, 482 bp, Leptosphaeria maculans
  source 1..482: mol_type = unassigned DNA
atgttaaaacctacaaaggtgatccaaatcttgtttctattatttaccgcattttttgcacgaacgtgcgctcttgaatgccacgctgttgctttctcttctgatcaccaattttctcttggtcgtaatgaggactgtaacttatactgcagcaaaaattcaatgcttagcatcttttcaagggttctgcctttagacgacccctgccaaggggatgatggatttagtagtcttacagccacgatcgatcaaataagttgtgatacaggatgtcaatgccgttgctctgttcacgccactgcttggcgagtccacaagtctggcaagcgatattcaagaagaactggctgggtatcttgcaatctagaagattatgttgctaggataactgggaggccttttataccagttaatggagcactacacgaatatttctcccgcggatttgtgtccaaagacgaggtacactgcgatcatcagta

SEQ ID NO: 41 - DNA, 410 bp, Leptosphaeria maculans
  source 1..410: mol_type = unassigned DNA
cttgaatgccacgctgttgctttctcttctgatcaccaattttctcttggtcgtaatgaggactgtaacttatactgcagcaaaaattcaatgcttagcatcttttcaagggttctgcctttagacgacccctgccaaggggatgatggatttagtagtcttacagccacgatcgatcaaataagttgtgatacaggatgtcaatgccgttgctctgttcacgccactgcttggcgagtccacaagtctggcaagcgatattcaagaagaactggctgggtatcttgcaatctagaagattatgttgctaggataactgggaggccttttataccagttaatggagcactacacgaatatttctcccgcggatttgtgtccaaagacgaggtacactgcgatcatcagta

SEQ ID NO: 42 - DNA, 4545 bp, Brassica napus
  source 1..4545: mol_type = unassigned DNA
atgttaaacatgagctcttacaatccttcttcccttatgattatcttctctctgctatttttcttggtctttgatctcacttttgccgggtcttgtcctaacacttgtggaggaatcgacatcccgtacccatttggaatcggggagggatgctatctgaataaatggtacgaaattaaatgtgatcataataactctctttcagtttcagggaagcgtgtccctattctttccttaatagccaaggaagttgtggatatctctcttccggaatcacttggctcgtatggttattattctcgcatgccatacgggaaaattcacataaaacacccaatagcttcaacggggtgctccagcaacaaacaagagtctagatcacttttgaatttgacagggagtcctttttacgtcggtgtcagtaataaactgataggtattggttgcaacagcacagcctcgttgacgaatgtcgagccaagcatcattggatgcttctccagttgcctggttcttactgctacaaaaaattatctagcgccagtcgattgtaatactacgaatgactacgggtattgtggtgaaagaagctgcaacggtaatggatgctgcaaggcaagcatgtccggtagtattcaacaggttgttggtgtcagaatagatgacaacgccaccacaacaggatggtgtaaagttgctttcttgtcaaatgaggaatacactttgtcagacagttctgatccagatcgggtatttgctaaacgatattcaacagttgagctaggatggttcatccatacaacgaatctttccttcttaggctctctaaaatgtgcaactatgaaagaatacctaaatctaagtcgaaactcatatgaatcgacttacggaataggttgtgcatgcgaccataactcctattttcctaactatgcaagttgcagctgcaatagtggctacatgggcaacccatacattctgaatggatgtaaagataaaaatgaatgcactgaaaaagacttgtactgtggggaggacgccacttgtgtcaatttgcaaggatcctataaatgtgtgtatagaaattaccggcttgctatagggatttgtgcaagtttcggctcattgatctttattggtggaatatgttggttatacaagtttattacaaaacaaaggaggttaaaccaaaagaagaagttcttcaagcgtaacggaggcttattgttgcaacaacaattaacttcaacccaaggcagcaatgaaaagacaagagttttcagctcaagagagctggaaaatgccactgagaactttggtttgactagaatacttggccagggaggtcaaggtaccgtctacaagggaatgcttgttgatggaagaattgtagcagtgaaaaagtccaaagtcgtggatgaagacaagctggaagagttcatcaatgaggttgtcattctctcccagatcaaccacagaaacatcgtgaagcttttagggtgttgccttgagacagatgttcctattctcgtctatgagtttattcccaatggaaaccttttcgagcatcttcatgaagatgagtcagataataatacattgaccacttgggaagtgcgtctccgcattgctatagatattgcaggagctctttcatatttgcattcagctgcttcatctccaatatatcacagggacatcaagtctacaaacataatgttggacgagaaatatcgagctaaagtgtctgattttggcacttcaagaacggtaacggtggatcatacacaccttacaacagtagtgtcaggtacagtgggatatgtggatcctgagtatttccagtctagtcagttcacagataaaagcgatgtttacagctttggagtggtgcttgttgagcttattaccggagaaaaatcagtttccttcttgcggtctcaagaaaataggacactggcaacttacttccttcgtgcagtgaaagagaataggctgttcgacattatcgatgcacgaatcagagatggctgcaagctgagtcaagtgaatgcaaccgcaaaccttgcaaggaagtgtttgaatctgaaagggaggaaacggccaagtatgagagaaacttccatggaattggagaaaattcgttcgtcttctgtggatatgaaactacatgagcatggtagtgaaaacaaggaagacaataaaaaaggagttgtggatgctaacataggggtacaatcatggaacaacaatactgttactgctccaacttctcaatacgacgtagatctcaccgttgccgggtcttgtcctagcaaatgtggaggaatcgacatcccatacccatttggaatcgggaagggctgctatctcaataaatggtatgaaattaaatgtgatcacaataactctttttcagtttcagggaagcctgtccctgttctttcaatacccgacaaggaagttgttaatatctctcttccgagtcatgtccactcagattatcctattcgcatgacatacggggcagttcgcataaaataccaaataacttcaaggggatgctccagcaacgaacaagagcctacatcacttttgaatttgatggggagccctttttacgttgctgacagtaatacaatgatagcgattggttgcaacaacagggcatcgctgacaaatatggagccaagtatcgtgggatgcttctccagttgcctctcaacaaaccatattgcttcgaatgattatctagacacagtcaattgttatagtaagagtgactccgatgacgcctacgagtactgtagtgaaagaagatatataaatgaaacaagctgcaacggtaatggatgctgcaaggcaagcatgcccagtagtgatcaacaagttgttggtgtcagaatagatgataactccgccgcaacaagagggtgtaaagttgccttcttaacagatgaggcctactctttattagacggaactgatccaaaactgcttcttgctaaacgatattctacagttgagctacagtggttcattgacacaacgaatctttcgtttgtagactctctaagatgcataactatgaaagagtacctacctataagtcgacaatactcacatggaatgaataactatgcaagttgtgcatgcgactttaattcttctgttcctaactacgcaagctgcagctgcaatattggttacaggggcaacccatacattctgggtggatgcaaagatattagtagtgaatgccctaattgcgtgtatagatatcacgtgcagcttgtaatagggattggttcaagtttcggctcattgattttcatttgtggaatatacggattatacagacttattagaaaacaaaggagtttaaaacaaaagaagaagttcttcaagcgtaatggaggcttgttgttgcaacaacaattaacttcaacccaaggcaacgttgaaaagacaagagtgttcagctcgagagagctggaaaaggccaccgaaaacttcagtttaactagaatacttggccaaggcggtcaaggaaccgtctacaaaggaatgcttgttgatggaagaattgtagcagtgaaaaagtccaaggtggtggatgaagacaagttggaagagttcatcaacgaggttgtcattctgtcgcagatcaaccacagaaacattgtcaaacttttagggtgttgcctcgagacagatgtcccggttctcgtctacgagtttattcccaatggtaacctttttgaacatcttcatgaagatgagtcagacgatcacacgataactacttgggaagtgcgtctccgcattgctattgatatcgcaggagctctttcatatttgcattcagccgcatcatctccaatatatcatagggacgtcaagtctacaaacataatgttggatgagaagtatcgagctaaagtgtctgattttggcacttcaagaacagtaacggtggatcatacacaccttacaacggtagtttcaggtacggtggggtatgtggatcctgagtatttccagtctagccagttcacggacaaaagtgatgtttacagctttggtgtggtgcttgcggagcttatcaccggagaaaaatcagtttctttcttgcggtctcaagaaaataggacattggcaacttatttctttcttgcgatgaaagagaataggctgcttgacattatcgatgcacggatcagagatggttgcaagctgaatcaagtgactgcaacggcaaatctcgcaaggaagtgtttgaatctgaaaggaaggaagcggccaagtatgcgagaagtgtccatggaattggagaaaattcgtgggtcttcggtggatatggaactacatgagtgtggtagtgaaaacgaggaagagaatgaagaacaaattgtggatgctaacatagagttacaatcatggaacaacactgccgttagtgctccaacttctcagtacgacgtcggtacatcatctttgtcaatgtcagatgttgagcctctatttccttttcaaacacggtaa

SEQ ID NO: 43 - protein, 1514 aa, Brassica napus
MLNMSSYNPSSLMIIFSLLFFLVFDLTFAGSCPNTCGGIDIPYPFGIGEGCYLNKWYEIKCDHNNSLSVSGKRVPILSLIAKEVVDISLPESLGSYGYYSRMPYGKIHIKHPIASTGCSSNKQESRSLLNLTGSPFYVGVSNKLIGIGCNSTASLTNVEPSIIGCFSSCLVLTATKNYLAPVDCNTTNDYGYCGERSCNGNGCCKASMSGSIQQVVGVRIDDNATTTGWCKVAFLSNEEYTLSDSSDPDRVFAKRYSTVELGWFIHTTNLSFLGSLKCATMKEYLNLSRNSYESTYGIGCACDHNSYFPNYASCSCNSGYMGNPYILNGCKDKNECTEKDLYCGEDATCVNLQGSYKCVYRNYRLAIGICASFGSLIFIGGICWLYKFITKQRRLNQKKKFFKRNGGLLLQQQLTSTQGSNEKTRVFSSRELENATENFGLTRILGQGGQGTVYKGMLVDGRIVAVKKSKVVDEDKLEEFINEVVILSQINHRNIVKLLGCCLETDVPILVYEFIPNGNLFEHLHEDESDNNTLTTWEVRLRIAIDIAGALSYLHSAASSPIYHRDIKSTNIMLDEKYRAKVSDFGTSRTVTVDHTHLTTVVSGTVGYVDPEYFQSSQFTDKSDVYSFGVVLVELITGEKSVSFLRSQENRTLATYFLRAVKENRLFDIIDARIRDGCKLSQVNATANLARKCLNLKGRKRPSMRETSMELEKIRSSSVDMKLHEHGSENKEDNKKGVVDANIGVQSWNNNTVTAPTSQYDVDLTVAGSCPSKCGGIDIPYPFGIGKGCYLNKWYEIKCDHNNSFSVSGKPVPVLSIPDKEVVNISLPSHVHSDYPIRMTYGAVRIKYQITSRGCSSNEQEPTSLLNLMGSPFYVADSNTMIAIGCNNRASLTNMEPSIVGCFSSCLSTNHIASNDYLDTVNCYSKSDSDDAYEYCSERRYINETSCNGNGCCKASMPSSDQQVVGVRIDDNSAATRGCKVAFLTDEAYSLLDGTDPKLLLAKRYSTVELQWFIDTTNLSFVDSLRCITMKEYLPISRQYSHGMNNYASCACDFNSSVPNYASCSCNIGYRGNPYILGGCKDISSECPNCVYRYHVQLVIGIGSSFGSLIFICGIYGLYRLIRKQRSLKQKKKFFKRNGGLLLQQQLTSTQGNVEKTRVFSSRELEKATENFSLTRILGQGGQGTVYKGMLVDGRIVAVKKSKVVDEDKLEEFINEVVILSQINHRNIVKLLGCCLETDVPVLVYEFIPNGNLFEHLHEDESDDHTITTWEVRLRIAIDIAGALSYLHSAASSPIYHRDVKSTNIMLDEKYRAKVSDFGTSRTVTVDHTHLTTVVSGTVGYVDPEYFQSSQFTDKSDVYSFGVVLAELITGEKSVSFLRSQENRTLATYFFLAMKENRLLDIIDARIRDGCKLNQVTATANLARKCLNLKGRKRPSMREVSMELEKIRGSSVDMELHECGSENEEENEEQIVDANIELQSWNNTAVSAPTSQYDVGTSSLSMSDVEPLFPFQTR

SEQ ID NO: 44 - DNA, 2331 bp, Brassica napus
  source 1..2331: mol_type = unassigned DNA
atgttaaacatgagctcttataatccttcttcccttttaattattttctctctgctatttttgttgatcttagatctcaccgttgccgggtcttgtcctagcaaatgtggaggaatcgacatcccatacccatttggaatcgggaagggctgctatctcaataaatggtatgaaattaaatgtgatcacaataactctttttcagtttcagggaagcctgtccctgttctttcaatacccgacaaggaagttgttaatatctctcttccgagtcatgtccactcagattatcctattcgcatgacatacggggcagttcgcataaaataccaaataacttcaaggggatgctccagcaacgaacaagagcctacatcacttttgaatttgatggggagccctttttacgttgctgacagtaatacaatgatagcgattggttgcaacaacagggcatcgctgacaaatatggagccaagtatcgtgggatgcttctccagttgcctctcaacaaaccatattgcttcgaatgattatctagacacagtcaattgttatagtaagagtgactccgatgacgcctacgagtactgtagtgaaagaagatatataaatgaaacaagctgcaacggtaatggatgctgcaaggcaagcatgcccagtagtgatcaacaagttgttggtgtcagaatagatgataactccgccgcaacaagagggtgtaaagttgccttcttaacagatgaggcctactctttattagacggaactgatccaaaactgcttcttgctaaacgatattctacagttgagctacagtggttcattgacacaacgaatctttcgtttgtagactctctaagatgcataactatgaaagagtacctacctataagtcgacaatactcacatggaatgaataactatgcaagttgtgcatgcgactttaattcttctgttcctaactacgcaagctgcagctgcaatattggttacaggggcaacccatacattctgggtggatgcaaagatattagtagtgaatgccctaattgcgtgtatagatatcacgtgcagcttgtaatagggattggttcaagtttcggctcattgattttcatttgtggaatatacggattatacagacttattagaaaacaaaggagtttaaaacaaaagaagaagttcttcaagcgtaatggaggcttgttgttgcaacaacaattaacttcaacccaaggcaacgttgaaaagacaagagtgttcagctcgagagagctggaaaaggccaccgaaaacttcagtttaactagaatacttggccaaggcggtcaaggaaccgtctacaaaggaatgcttgttgatggaagaattgtagcagtgaaaaagtccaaggtggtggatgaagacaagttggaagagttcatcaacgaggttgtcattctgtcgcagatcaaccacagaaacattgtcaaacttttagggtgttgcctcgagacagatgtcccggttctcgtctacgagtttattcccaatggtaacctttttgaacatcttcatgaagatgagtcagacgatcacacgataactacttgggaagtgcgtctccgcattgctattgatatcgcaggagctctttcatatttgcattcagccgcatcatctccaatatatcatagggacgtcaagtctacaaacataatgttggatgagaagtatcgagctaaagtgtctgattttggcacttcaagaacagtaacggtggatcatacacaccttacaacggtagtttcaggtacggtggggtatgtggatcctgagtatttccagtctagccagttcacggacaaaagtgatgtttacagctttggtgtggtgcttgcggagcttatcaccggagaaaaatcagtttctttcttgcggtctcaagaaaataggacattggcaacttatttctttcttgcgatgaaagagaataggctgcttgacattatcgatgcacggatcagagatggttgcaagctgaatcaagtgactgcaacggcaaatctcgcaaggaagtgtttgaatctgaaaggaaggaagcggccaagtatgcgagaagtgtccatggaattggagaaaattcgtgggtcttcggtggatatggaactacatgagtgtggtagtgaaaacgaggaagagaatgaagaacaaattgtggatgctaacatagagttacaatcatggaacaacactgccgttagtgctccaacttctcagtacgacgtcggtacatcatctttgtcaatgtcagatgttgagcctctatttccttttcaaacacggtaa

SEQ ID NO: 45 - protein, 776 aa, Brassica napus
MLNMSSYNPSSLLIIFSLLFLLILDLTVAGSCPSKCGGIDIPYPFGIGKGCYLNKWYEIKCDHNNSFSVSGKPVPVLSIPDKEVVNISLPSHVHSDYPIRMTYGAVRIKYQITSRGCSSNEQEPTSLLNLMGSPFYVADSNTMIAIGCNNRASLTNMEPSIVGCFSSCLSTNHIASNDYLDTVNCYSKSDSDDAYEYCSERRYINETSCNGNGCCKASMPSSDQQVVGVRIDDNSAATRGCKVAFLTDEAYSLLDGTDPKLLLAKRYSTVELQWFIDTTNLSFVDSLRCITMKEYLPISRQYSHGMNNYASCACDFNSSVPNYASCSCNIGYRGNPYILGGCKDISSECPNCVYRYHVQLVIGIGSSFGSLIFICGIYGLYRLIRKQRSLKQKKKFFKRNGGLLLQQQLTSTQGNVEKTRVFSSRELEKATENFSLTRILGQGGQGTVYKGMLVDGRIVAVKKSKVVDEDKLEEFINEVVILSQINHRNIVKLLGCCLETDVPVLVYEFIPNGNLFEHLHEDESDDHTITTWEVRLRIAIDIAGALSYLHSAASSPIYHRDVKSTNIMLDEKYRAKVSDFGTSRTVTVDHTHLTTVVSGTVGYVDPEYFQSSQFTDKSDVYSFGVVLAELITGEKSVSFLRSQENRTLATYFFLAMKENRLLDIIDARIRDGCKLNQVTATANLARKCLNLKGRKRPSMREVSMELEKIRGSSVDMELHECGSENEEENEEQIVDANIELQSWNNTAVSAPTSQYDVGTSSLSMSDVEPLFPFQTR

SEQ ID NO: 46 - DNA, 2615 bp, Brassica napus
  source 1..2615: mol_type = unassigned DNA
caaaacccagtcaccttttactaaagaaaatcatacaccggaaataacaaattttcttgagaagatgttaaacatgagctcttataatccttcttcccttttaattattttctctctgctatttttgttgatcttagatctcaccgttgccgggtcttgtcctagcaaatgtggaggaatcgacatcccatacccatttggaatcgggaagggctgctatctcaataaatggtatgaaattaaatgtgatcacaataactctttttcagtttcagggaagcctgtccctgttctttcaatacccgacaaggaagttgttaatatctctcttccgagtcatgtccactcagattatcctattcgcatgacatacggggcagttcgcataaaataccaaataacttcaaggggatgctccagcaacgaacaagagcctacatcacttttgaatttgatggggagccctttttacgttgctgacagtaatacaatgatagcgattggttgcaacaacagggcatcgctgacaaatatggagccaagtatcgtgggatgcttctccagttgcctctcaacaaaccatattgcttcgaatgattatctagacacagtcaattgttatagtaagagtgactccgatgacgcctacgagtactgtagtgaaagaagatatataaatgaaacaagctgcaacggtaatggatgctgcaaggcaagcatgcccagtagtgatcaacaagttgttggtgtcagaatagatgataactccgccgcaacaagagggtgtaaagttgccttcttaacagatgaggcctactctttattagacggaactgatccaaaactgcttcttgctaaacgatattctacagttgagctacagtggttcattgacacaacgaatctttcgtttgtagactctctaagatgcataactatgaaagagtacctacctataagtcgacaatactcacatggaatgaataactatgcaagttgtgcatgcgactttaattcttctgttcctaactacgcaagctgcagctgcaatattggttacaggggcaacccatacattctgggtggatgcaaagatattagtagtgaatgccctaattgcgtgtatagatatcacgtgcagcttgtaatagggattggttcaagtttcggctcattgattttcatttgtggaatatacggattatacagacttattagaaaacaaaggagtttaaaacaaaagaagaagttcttcaagcgtaatggaggcttgttgttgcaacaacaattaacttcaacccaaggcaacgttgaaaagacaagagtgttcagctcgagagagctggaaaaggccaccgaaaacttcagtttaactagaatacttggccaaggcggtcaaggaaccgtctacaaaggaatgcttgttgatggaagaattgtagcagtgaaaaagtccaaggtggtggatgaagacaagttggaagagttcatcaacgaggttgtcattctgtcgcagatcaaccacagaaacattgtcaaacttttagggtgttgcctcgagacagatgtcccggttctcgtctacgagtttattcccaatggtaacctttttgaacatcttcatgaagatgagtcagacgatcacacgataactacttgggaagtgcgtctccgcattgctattgatatcgcaggagctctttcatatttgcattcagccgcatcatctccaatatatcatagggacgtcaagtctacaaacataatgttggatgagaagtatcgagctaaagtgtctgattttggcacttcaagaacagtaacggtggatcatacacaccttacaacggtagtttcaggtacggtggggtatgtggatcctgagtatttccagtctagccagttcacggacaaaagtgatgtttacagctttggtgtggtgcttgcggagcttatcaccggagaaaaatcagtttctttcttgcggtctcaagaaaataggacattggcaacttatttctttcttgcgatgaaagagaataggctgcttgacattatcgatgcacggatcagagatggttgcaagctgaatcaagtgactgcaacggcaaatctcgcaaggaagtgtttgaatctgaaaggaaggaagcggccaagtatgcgagaagtgtccatggaattggagaaaattcgtgggtcttcggtggatatggaactacatgagtgtggtagtgaaaacgaggaagagaatgaagaacaaattgtggatgctaacatagagttacaatcatggaacaacactgccgttagtgctccaacttctcagtacgacgtcggtacatcatctttgtcaatgtcagatgttgagcctctatttccttttcaaacacggtaataatttcaactttgcataccacgacattatctagtgcctgccttttgctttcccgtcctagtttggtgtttctgtatgtgtgatgtatttcttttgtgtaataaatacctatgattgtgtaataaaaggatctcgtttgtaacacagaagtttacattctgataagccagtctttaggctgttgttagccattgcattagagggacaaacataaaataat

SEQ ID NO: 47 - DNA, 2615 bp, Brassica napus; identical to SEQ ID NO: 46
  source 1..2615: mol_type = unassigned DNA